(19) United States
(12) Patent Application Publication
(10) Pub. No.: US 2011/0098303 A1
(43) Pub. Date: Apr. 28, 2011

(54) Method of promoting nail growth using thyromimetic compounds

(76) Inventors: Niall S. Doherty, Stonington, CT (US); Tanya Parkinson, Shepherdswell (GB)

(21) Appl. No.: 10/715,874

(22) Filed: Nov. 18, 2003

Related U.S. Application Data
(60) Provisional application No. 60/429,040, filed on Nov. 25, 2002.

(51) Int. Cl. A61K 31/195; A61K 31/53; A61K 31/41; A61K 31/216; A61K 31/426; A61K 31/4245; A61K 31/4196; A61K 31/404; A61K 31/496; A61K 31/4418; A61P 43/00; B65D 69/00

(52) U.S. Cl. 514/242; 514/563; 514/381; 514/533; 514/369; 514/364; 514/384; 514/419; 514/383; 514/254.07; 514/345; 206/570

(57) ABSTRACT

The present invention provides methods and compositions for increasing the rate of nail growth in mammals, especially humans, using thyromimetic compounds.

### CROSS-REFERENCE TO RELATED APPLICATION

This application claims the benefit of U.S. Provisional Patent Application No. 60/429,040 filed Nov. 25, 2002.

### FIELD OF THE INVENTION

The present invention provides methods and compositions for promoting nail growth in mammals, especially humans, using thyromimetic compounds, as described below.

### BACKGROUND OF THE INVENTION

Humans have a nail at the tip of each finger and toe. Nails provide protection to the delicate structures of the fingertip. In addition, fingernails play an important role in picking up and manipulating objects, scratching, providing leverage (e.g., opening cans) and have a very important role in appearance. Disfigured or discolored nails can have a major impact on attractiveness that can impact lifestyle in important ways. For example, in certain professions, e.g., physicians or dentists, ugly nails would be a significant disadvantage that would affect income. In addition, attractive nails are a component of personal attractiveness and a considerable amount of time and money is spent on maintenance and adornment of nails.

Because of their roles in protecting the underlying soft tissue, their use in providing leverage, their use in scratching and their exposed location, nails are subject to physical trauma. Some of this trauma can be self-inflicted by the habit of nail biting. Nail injuries are not repaired; it is necessary for the nail to be replaced by the growth of new undamaged nail. Growth occurs only in the nail matrix, an area below the base of the nail. The new nail material slides forward on the nail bed and as the tip of the nail extends beyond the end of the finger, it is cut or worn away. Nails grow slowly (fingernails grow 0.1 mm per day) and it takes four to six months to completely replace a fingernail. Toenails grow more slowly and are replaced in eight to twelve months. Therefore, any damage to a nail normally takes considerable time to grow out, and during this period, the nail can be unattractive and inconvenient to use by the patient. See U. Runne and C. E. Orfanos, “The Human Nail,” Curr. Probl. Derm., vol. 9, pp. 102-149 (Karger, Basel 1981), for a general discussion on the structure, growth and pathological changes to the human nail. See also the following references on the nail: R. Dawber and R. Baran, “Nail Growth,” CUTIS 39, 99-103 (1987); D. L. Moffitt and D. A. R. de Berker, “Yellow nail syndrome: the nail that grows half as fast grows twice as thick,” Clinical and Experimental Dermatology, 25, 21-23 (2000); and N. Orentreich et al., “The Effect of Aging on the Rate of Linear Nail Growth,” J. Invest. Dermatol., 73: 126-130 (1979).

Infection can also damage nails. The most common type of nail infection is fungal. In fact, 50% of all nail disorders result from fungal infection. Fungal infections of nails cause disfiguring changes in nail color and shape and are particularly difficult to treat. Current antifungal agents kill metabolically active organisms, but have no effect on fungal spores. Therefore, even though a relatively short treatment period may kill all the active fungi, once treatment is stopped the spores can initiate a new round of infection. As a result, routine therapy lasts four to six months in an effort to allow all the spores to be eliminated through growth and removal of the spore-containing nail. Despite this, failure to eradicate the infection is common and patients are generally unhappy with the extended period during which they have disfiguring changes caused by the fungus.

An agent that accelerates nail growth would accelerate the recovery to normal appearance of nails injured by physical trauma or infection. In addition, by accelerating the growth of nails, the rate of clearance of spores would also be accelerated allowing shorter treatment times with antifungal agents.

In patients with thyrotoxicosis (elevated circulating levels of thyroid hormone), it has frequently been observed that nail growth is accelerated. However, the nails that are formed in thyrotoxicosis are brittle and may separate from the underlying nail bed (onycholysis). However, there is evidence that suggests that local effects of thyroid hormone can be very different from the effects of thyroid hormone distributed throughout the body. Specifically, systemically administered tri-iodothyronine (T3, the biologically active form of thyroid hormone) inhibits the growth of hair in experiments in mice. In contrast, when administered locally, directly to the skin, T3 produces a dramatic acceleration of hair growth. See J. D. Safer, L. M. Fraser, M. Hoa and M. F. Holick, “Intraperitoneal and Topical Triiodothyronine Have Opposing Effects on Mouse Skin,” Abstract P1-530 for The Endocrine Society 83^{rd} Annual Meeting, Jun. 20-23, 2001; and J. D. Safer, et al., “Topical Triiodothyronine Stimulates Epidermal Proliferation, Dermal Thickening and Hair Growth in Mice and Rats,” Thyroid 11 (8): 717-24 (August 2001).

Two naturally occurring thyroid hormones, namely, thyroxine or 3,5,3′,5′-tetraiodo-L-thyronine (commonly referred to as “T₄”), and thyronine or 3,5,3′-triiodo-L-thyronine (commonly referred to as “T3”), are shown below:

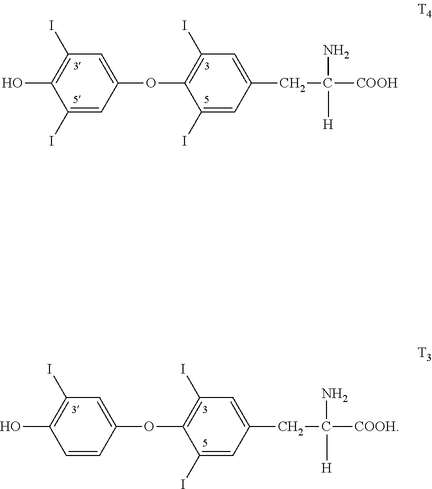

T₃ is the more biologically active of the two and, as will be appreciated from the structural formulae provided above, differs from T₄ by the absence of the 5′ iodine. T₃ may be produced directly from the thyroid gland or, in peripheral tissues, by the removal of the 5′ iodine by deiodinase enzymes. Thyromimetic analogs are often designed to be structurally similar to T₃. In addition, naturally occurring metabolites of T₃ are known.

Interestingly, it is known that the thyroid hormone, thyroxine (“T₄”), converts to triiodo-thyronine (“T₃”) in human skin by deiodinase I, a selenoprotein. Selenium deficiency causes a decrease in T₃ levels due to a decrease in deiodinase I activity; this reduction in T₃ levels is strongly associated with hair loss. Consistent with this observation, hair growth is a reported side effect of administration of T₄. See, for example, Berman, “Periperal Effects of L-Thyroxine on Hair Growth and Coloration in Cattle,” Journal of Endocrinology, Vol. 20, pp. 282-292 (1960); and Gunaratnam, “The Effects of Thyroxine on Hair Growth in the Dog,” J. Small Anim. Pract, Vol. 27, pp. 17-29 (1986). Furthermore, T₃ and T₄ have been the subject of several patent publications relating to treatment of hair loss. See, e.g., German patent 1,617,477; British patent 2,138,286; and WO 96/25943. Thus, it is known that thyroid hormone can exert positive effects on hair growth; however, administration of T₃ and/or T₄ to treat hair loss is not practicable because these thyroid hormones are known to cause adverse side effects, such as inducing significant cardiotoxicity or adversely affecting bone mineral density and lean body mass. See, e.g., U.S. Pat. No. 5,284,971; and U.S. Pat. No. 5,061,798.

Hair growth enhancement has been seen with synthetic molecules, which also stimulate thyroid hormone receptors. Published International patent application WO 00/72810 discloses methods of treating hair loss using certain sulfonyl thyromimetic compounds. Published International patent application WO 00/72811 discloses methods of treating hair loss using certain compounds, such as substituted phenoxy-benzoic acid compounds, described therein. Published International patent application WO 00/72812 discloses methods of treating hair loss using certain diphenylether derivatives. Published International patent application WO 00/72813 discloses methods of treating hair loss using certain diphenylmethane derivatives. Published International patent application WO 00/72920 discloses certain substituted biaryl ether compounds and compositions for treating hair loss. Published International patent application WO 00/73292 discloses certain biaryl compounds and compositions for treating hair loss.

Commonly assigned, published International patent application WO 00/51971 and commonly assigned, published European patent application EP 1 033 364 disclose certain oxamic acids and derivatives thereof as thyroid receptor ligands. Commonly assigned, published European Patent Application EP 1 088 819 discloses certain 6-azauracil derivatives as thyroid receptor ligands. Commonly assigned, published European Patent Application EP 1 127 882 discloses certain tetrazole compounds as thyroid receptor ligands. Commonly assigned, published European Patent Application EP 1 148 054 discloses certain thiazolidinedione, oxadiazolidinedione and triazolone compounds, which are thyroid receptor ligands. Commonly assigned, published International patent application WO 0102692 discloses certain malonamic acids and derivatives thereof as thyroid receptor ligands. Commonly assigned, U.S. patent application Ser. No. 10/255,180, filed Sep. 24, 2002, discloses certain indole carboxylic acids as thyroid receptor ligands. Commonly assigned, U.S. patent application Ser. No. 10/160,516, filed May 30, 2002, the use of certain thyromimetic compounds to promote hair growth.

U.S. Pat. No. 6,221,911 discloses the use of topically applied thyroid hormone compounds and thyroid hormone-like compounds to improve the appearance of the skin and underlying subcutaneous fat and improve certain medical skin conditions when applied topically.

The following are recent articles on thyroid hormone receptors (TRs): M. K. Ahsan et al., J. Med. Invest. 44: 179-184, 1998, studied the immunohistochemical localization of thyroid hormone receptors (TRs) in human scalp skin and concluded that the results demonstrated the presence of thyroid hormone nuclear receptors in human hair follicles. N. Billoni et al., British Journal of Dermatology 2000:142:645-652, established that TRβ1 was the predominant form of TR expressed in the human hair follicle. C. C. Thompson and M. C. Bottcher, Proc. Natl. Acad. Sci. USA, Vol. 94, pp. 8527-8532, August 1997, found that the product of a thyroid hormone-responsive gene, the lack of which confers a hairless phenotype, interacts with thyroid hormone receptors. A. G. Messenger, British Journal of Dermatology, 142, 631-635, 2000, discussed the relationship between thyroid hormone and hair growth. V. L. Malloy, et al., Abstract titled “Effect of Topically Applied Thyroid Hormone on Androgen Dependent Models of the Pilo-Sebaceous Apparatus,” Clinical Research, Vol. 36, No. 5, page 814A (1998), observed an inhibitory effect on testosterone induced alopecia after treatment with topical T₃ in the AGA mouse, a model for androgen dependent hair loss.

There are many similarities between hair and nail. Both are keratin-containing structures formed from epidermal cells. However, there are also fundamental differences between hair and nail. In particular, hair growth is cyclical as the hair follicle goes through cycles of growth, and the whole hair fiber is shed in each cycle. There is no such cyclical growth in nails and the whole nail is not normally shed, although the growth rate of nail may be affected by a number of environmental and endogenous factors.

According to the present invention, the local application of compounds that mimic all or some of the effects of the thyroid hormones (thyromimetics), directly to the region of the nail where growth occurs will cause acceleration in the growth rate of the nail and the return of the nail to a normal-looking, attractive appearance. In addition, local rather than systemic administration of thyromimetic compounds to certain tissues, including those in the nail matrix which control nail growth, will spare other tissues, such as the heart, from exposure to such compounds, thereby avoiding the side effects associated with thyromimetic treatment.

### SUMMARY OF THE INVENTION

The present invention relates to methods for increasing the rate of nail growth in mammals comprising administering thyromimetic compounds, as described herein.

The present invention also relates to the use of certain thyromimetic compounds, as described below, for the manufacture or preparation of a medicament for increasing the rate of nail growth in mammals.

More particularly, the present invention provides such methods wherein the compounds are cardiac-sparing.

More particularly, the present invention provides such methods wherein the treatment is the acceleration of nail growth following infection of the nail or damage to the nail by any means, including chemical or physical damage.

More particularly, the present invention provides such methods wherein the mammal is a human being.

More particularly, the present invention provides such methods wherein the compounds are administered locally, and, preferably, topically.

More particularly, the present invention provides such methods, which further comprise the administration of an effective amount of an antifungal agent. These antifungal agents may be administered systemically, such as orally, or locally, and preferably, topically.

In addition, the present invention provides topical compositions for promoting the rate of nail growth, which comprise an effective amount of certain compounds, as described below, and pharmaceutically acceptable carriers.

More particularly, the present invention provides such compositions wherein the topical composition is in the form of a lotion, cream, ointment, lacquer (e.g., nail polish), paste, gel, spray, aerosol, bandages or kit. Also, the present invention provides such compositions where the topical composition is incorporated into an artificial nail where the artificial nail provides a cosmetically attractive appearance as the nail grows out plus acts as a slow-release delivery system for the topical composition.

The present invention also provides such compositions, which further comprise an effective amount of an agent for increasing the rate of nail growth.

In addition, the present invention provides kits for increasing the rate of nail growth in a mammal, the kit comprising:

a) a first pharmaceutical composition comprising a compound as described below;

b) a second pharmaceutical composition comprising an additional compound which is an antibacterial agent or an antifungal agent; and

c) a container.

More particularly, the present invention provides such kits wherein the additional compound is an antibacterial agent or an antifungal agent.

### DETAILED DESCRIPTION OF THE INVENTION

The present invention relates to methods for increasing the rate of nail growth in mammals, comprising administering thyromimetic compounds, as described below.

Preferred mammals include humans, companion animals such as dogs, cats and horses, and livestock such as cattle, swine and sheep. Particularly preferred mammals include humans.

Preferably, in the methods of the present invention, the compounds are administered topically.

The preferred compounds useful in the methods of the present invention are cardiac-sparing. The term “cardiac-sparing” as used herein means that, at the dosages required for hair growth, the compounds useful in the methods of the present invention do not produce any observable cardiotoxicity in the mammal being treated.

All percentages, ratios and proportions used herein are by weight unless otherwise specified.

As used herein, “effective amount of a compound” means an amount that is effective to exhibit biological activity, preferably wherein the biological activity is increasing nail growth, at the site(s) of activity in a mammalian subject, without undue adverse side effects (such as undue toxicity, irritation or allergic response), commensurate with a reasonable benefit/risk ratio when used in the manner of the present invention.

The phrase “compound(s) useful in the methods of the present invention,” and the like, shall at all times be understood to include all active forms of such compounds, including, for example, the free form thereof, e.g., the free acid or base form, and also, all prodrugs, polymorphs, hydrates, solvates, tautomers, stereoisomers, e.g., diastereomers and enantiomers, and the like, and all pharmaceutically acceptable salts as described above, unless specifically stated otherwise. It will also be appreciated that suitable active metabolites of such compounds, in any suitable form, are also included herein.

The term “thyromimetic compound” refers to a compound which mimics all or some of the effects of the thyroid hormones by interaction with one or more forms of the thyroid hormone receptors, which activity can be confirmed by assays well known in the art, such as those set forth in published European patent application EP 1 088 819, published Apr. 4, 2001 (particularly page 53), which reference is incorporated by reference herein.

The term “onychomycosis” refers to a fungal infection of the nail unit, defined as the nail matrix, bed or plate.

By “local administration” is meant that the method of administration is confined to the area of the patient requiring treatment. It is not general or systemic. For example, in the present invention, local administration to the nail includes administration to the nail and/or immediately surrounding tissue.

Following are listed particular examples of thyromimetic compounds which may be used in practicing the present invention. It is understood that in the generic formulae employed below, the variables employed, e.g., “A”, “B”, “R¹”, “R²”, etc. have the meanings attributed to them only in the particular Roman numeral section in which they are found. Thus, the meaning attributed, for example, to “R¹” is different for the structures in section I than the meaning attributed to it in the other sections.

I. For example, the thyromimetic compounds useful in the methods of the present invention have the following formula, also described in commonly assigned, published International patent application WO 00/51971:

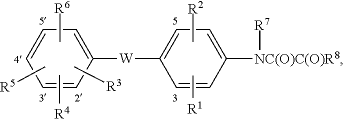

a prodrug thereof, a geometric or optical isomer thereof, or a pharmaceutically acceptable salt of said compound, said prodrug, or said isomer, wherein:

R¹, R² and R³ are each independently hydrogen, halogen, C₁₋₆ alkyl, trifluoromethyl, —CN, —OCF₃ or —OC₁₋₆ alkyl;

R⁴ is hydrogen, C₁₋₁₂ alkyl optionally substituted with one to three substitutents independently selected from Group Z, C₂₋₁₂ alkenyl, halogen, —CN, aryl, heteroaryl, C₃₋₁₀ cycloalkyl, heterocycloalkyl, —S(O)₂NR⁹R¹⁰, —C(O)NR⁹R¹⁰, —(C₁₋₆ alkyl)-NR⁹R¹⁰, —NR⁹C(O)R¹⁰, —NR⁹C(O)NR⁹R¹⁰, —NR⁹S(O)₂R¹⁰, —(C₁₋₆ alkyl)-OR¹¹, —OR¹¹ or —S(O)ₐR¹², provided that, where R⁵ is not fluoro, R⁴ is —S(O)₂NR⁹R¹⁰, —C(O)NR⁹R¹⁰, —(C₁₋₆ alkyl)-NR⁹R¹⁰, —NR⁹C(O)R¹⁰, —NR⁹C(O)NR⁹R¹⁰, —NR⁹S(O)₂R¹⁰, (C₁₋₆ alkyl)-OR¹¹, —OR¹¹ or —S(O)ₐR¹²;

or R³ and R⁴ may be taken together to form a carbocyclic ring A of the formula —(CH₂)_{b}— or a heterocyclic ring A selected from the group consisting of -Q-(CH₂)_{c}— and —(CH₂)ⱼ-Q-(CH₂)ₖ— wherein Q is O, S or NR¹⁷, wherein said carbocyclic ring A and said heterocyclic ring A are each independently optionally substituted with one or more substituents independently selected from C₁₋₄ alkyl, halide or oxo;

R⁵ is fluoro, hydroxy, C₁₋₄ alkoxy or OC(O)R⁹;

or R⁴ and R⁵ may be taken together to form a heterocyclic ring B selected from the group consisting of —CR⁹═CR¹⁰—NH—, —N═CR⁹—NH—, —CR⁹═CH—O— and —CR⁹═CH—S—;

R⁶ is hydrogen, halogen, C₁₋₄ alkyl or trifluoromethyl;

R⁷ is hydrogen or C₁₋₆ alkyl;

R⁸ is —OR⁹ or —NR¹⁹R²⁰;

R⁹ and R¹⁰ for each occurrence are independently (A) hydrogen, (B) C₁₋₁₂ alkyl optionally substituted with one or more substituents independently selected from Group V, (C) C₂₋₁₂ alkenyl, (D) C₃₋₁₀ cycloalkyl optionally substituted with one or more substituents independently selected from C₁₋₆ alkyl, C₂₋₅ alkynyl, C₃₋₁₀ cycloalkyl, —CN, —NR¹³R¹⁴, oxo, —OR¹⁸, —COOR¹⁸ or aryl optionally substituted with X and Y, (E) aryl optionally substituted with X and Y, or (F) het optionally substituted with X and Y;

or R⁹ and R¹⁰ for any occurrence may be taken together to form a heterocyclic ring C optionally further containing a second heterogroup selected from the group consisting of —O—, —NR¹³— and —S—, and optionally further substituted with one or more substituents independently selected from C₁₋₅ alkyl, oxo, —NR¹³R¹⁴, —OR¹⁸, —C(O)₂R¹⁸, —CN, —C(O)R⁹, aryl optionally substituted with X and Y, het optionally substituted with X and Y, C₅₋₆ spirocycloalkyl, and a carbocyclic ring B selected from the group consisting of 5-, 6-, 7- and 8-membered partially and fully saturated, and unsaturated carbocyclic rings, and including any bicyclic group in which said carbocyclic ring B is fused to a carbocyclic ring C selected from the group consisting of 5-, 6-, 7- and 8-membered partially and fully saturated, and unsaturated carbocyclic rings;

R¹¹ is C₁₋₁₂ alkyl optionally substituted with one or more substituents independently selected from Group V, C₂₋₁₂ alkenyl, C₃₋₁₀ cycloalkyl, trifluoromethyl, difluoromethyl, monofluoromethyl, aryl optionally substituted with X and Y, het optionally substituted with X and Y, —C(O)NR⁹R¹⁰ or —C(O)R⁹;

R¹² is C₁₋₁₂ alkyl optionally substituted with one or more substituents independently selected from Group V, C₂₋₁₂ alkenyl, C₃₋₁₀ cycloalkyl, aryl optionally substituted with X and Y, or het optionally substituted with X and Y;

R¹³ and R¹⁴ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl): hydroxy, —(C₁₋₄ alkyl)-halo, —(C₁₋₄ alkyl)-poly-halo, —(C₁₋₄ alkyl)-CONR¹⁵R¹⁶ or C₃₋₁₀ cycloalkyl;

R¹⁵ and R¹⁶ for each occurrence are independently hydrogen, C₁₋₆ alkyl, C₃₋₁₀ cycloalkyl or aryl optionally substituted with X and Y;

R¹⁷ is hydrogen, C₁₋₆ alkyl, —COR⁹ or —SO₂R⁹;

R¹⁸ is hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁵R¹⁸, —(C₁₋₄ alkyl) —(C₁₋₄ alkoxy) or C₃₋₁₀ cycloalkyl;

R¹⁹ is hydrogen or C₁₋₆ alkyl;

R²⁰ is hydrogen or C₁₋₆ alkyl;

W is O, S(O)_{d}, CH₂ or NR⁹;

Group Z is C₂₋₆ alkenyl, C₂₋₆ alkynyl, halogen, —CF₃, —OCF₃, hydroxy, oxo, —CN, aryl, heteroaryl, C₃₋₁₀ cycloalkyl, heterocycloalkyl, —S(O)ₐR¹², —S(O)₂NR⁹R¹⁰, —C(O)R⁹R¹⁰, and —NR⁹R¹⁰;

Group V is halogen, —NR¹³R¹⁴, —OR⁹, oxo, trifluoromethyl, —CN, C₃₋₁₀ cycloalkyl, aryl optionally substituted with X and Y, and het optionally substituted with X and Y;

het for each occurrence is a heterocyclic ring D selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring D is fused to a benzene ring or a heterocyclic ring E selected from the group consisting of 4-, 5-, 6-, 7- and 8-membered partially and fully saturated, and unsaturated, heterocyclic rings containing from one to four heteroatoms independently selected from the group consisting of N, O and S;

X and Y for each occurrence are independently (A) hydrogen, (B) halogen, (C) trifluoromethyl, (D) —OCF₃, (E) —CN, (F) C₁₋₆ alkyl optionally substituted with one or more substituents independently selected from the group consisting of halogen, —OCF₃, —CF₃ and phenyl, (G) C₁₋₆ alkoxy, (H) aryl optionally substituted with one or more substituents independently selected from the group consisting of halogen, —OCF₃, —CF₃, C₁₋₄ alkyl and C₁₋₄ alkoxy, (I) —C(O)₂R¹³, (J) —C(O)NR¹³R¹⁴, (K) —C(O)R¹³, (L) —NR¹³C(O)NR¹³R¹⁴ and (M) —NR¹³C(O)R¹⁴;
or X and Y for any occurrence in the same variable may be taken together to form (a) a carbocyclic ring D of the formula —(CH₂)ₑ— or (b) a heterocyclic ring F selected from the group consisting of —O(CH₂)_{f}O—, (CH₂)_{g}NH— and —CH═CHNH—;

a and d are each independently 0, 1 or 2;

b is 3, 4, 5, 6 or 7;

c, f, g, j and k are each independently 2, 3, 4, 5 or 6; and

e is 3, 4, 5, 6 or 7.

Specific thyromimetic compounds which may be used in the methods of the present invention include the following:
N-[3-chloro-4-(3-cyclopropylsulfamoyl-4-hydroxy-phenoxy)-5-methyl-phenyl]-oxamic acid;
N-[4-(3-cyclopropylsulfamoyl-4-hydroxy-phenoxy)-3,5-dimethyl-phenyl]-oxamic acid;
N-{4-[3-(cyclobutyl-methyl-carbamoyl)-4-hydroxy-phenoxy]-3,5-dimethyl-phenyl}-oxamic acid;
N-{3-chloro-4-[3-(cyclobutyl-methyl-carbamoyl)-4-hydroxy-phenoxy]-5-methyl-phenyl}-oxamic acid;
N-[4-(7-hydroxy-indan-4-yloxy)-3,5-dimethyl-phenyl]-oxamic acid;
N-{3,5-dichloro-4-[3-(cyclobutyl-methyl-carbamoyl)-4-hydroxy-phenoxy]-phenyl}-oxamic acid;
N-[3,5-dichloro-4-(3-cyclopentanesulfonyl-4-hydroxy-phenoxy)-phenyl]-oxamic acid;
N-[3,5-dichloro-4-(3-cyclopropylmethanesulfonyl-4-hydroxy-phenoxy)-phenyl]-oxamic acid;
N-[3,5-dichloro-4-(3-cyclobutylmethanesulfonyl-4-hydroxy-phenoxy)-phenyl]-oxamic acid;
N-[4-(3-cyclopropylmethanesulfonyl-4-hydroxy-phenoxy)-3,5-dimethyl-phenyl]oxamic acid;
N-[3-chloro-4-(3-cyclobutylmethanesulfonyl-4-hydroxy-phenoxy)-5-methyl-phenyl]oxamic acid;
N-[4-(3-cyclobutylmethanesulfonyl-4-hydroxy-phenoxy)-3,5-dimethyl-phenyl]oxamic acid;
N-[4-(3-cyclopentylmethanesulfonyl-4-hydroxy-phenoxy)-3,5-dimethyl-phenyl]-oxamic acid;
N-[3-chloro-4-(3-cyclopentylmethanesulfonyl-4-hydroxy-phenoxy)-5-methyl-phenyl]-oxamic acid;
N-[3,5-dichloro-4-(3-cyclopentylmethanesulfonyl-4-hydroxy-phenoxy)-phenyl]-oxamic acid;
N-[4-(3-cyclohexylmethanesulfonyl-4-hydroxy-phenoxy)-3,5-dimethyl-phenyl]-oxamic acid;
N-[3-chloro-4-(3-cyclohexylmethanesulfonyl-4-hydroxy-phenoxy)-5-methyl-phenyl]-oxamic acid;
N-[3,5-dichloro-4-(3-cyclohexylmethanesulfonyl-4-hydroxy-phenoxy)-phenyl]-oxamic acid;
N-[3,5-dichloro-4-[3-(4-fluoro-benzenesulfonyl)-4-hydroxy-phenoxy)-phenyl]-oxamic acid;
N-{4-[3-(4-fluoro-benzenesulfonyl)-4-hydroxy-phenoxy]-3,5-dimethyl-phenyl}-oxamic acid; and
N-{3-chloro-4-[3-(4-fluoro-benzenesulfonyl)-4-hydroxy-phenoxy]-5-methyl-phenyl}-oxamic acid.

II. The present invention also includes the use of the thyromimetic compounds of the following formula, also described in commonly assigned, published European patent application EP 1 033 364:

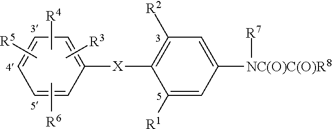

a prodrug thereof, a geometric or optical isomer thereof, or a pharmaceutically acceptable salt of said compound, said prodrug, or said isomer, wherein:

R¹ and R² are independently halogen, C₁₋₈ alkyl, —CN or C₁₋₈ perfluoroalkyl; provided that at least one of R¹ and R² is —CN;

R³ is hydrogen or C₁₋₈ alkyl;

R⁴ is halogen, C₁₋₈ perfluoroalkyl, C₁₋₈ alkyl, C₁₋₈ alkanoyl, hydroxy-(C₁₋₈ alkyl), aryl optionally substituted with Y and Z, aryl-(C₁₋₈ alkyl), carbocyclic aroyl optionally substituted with Y and Z, C₃₋₁₀ cycloalkyl optionally substituted with Y and Z, or C₃₋₁₀ cycloalkyl-(C₁₋₈ alkyl);

or R⁴ is the radical

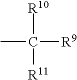

wherein: R⁹ is hydrogen, C₁₋₈ alkyl, aryl optionally substituted with Y and Z, aryl-(C₁₋₈ alkyl), C₃₋₁₀ cycloalkyl optionally substituted with Y and Z, or C₃₋₁₀ cycloalkyl-(C₁₋₈ alkyl); R¹⁰ is —OR¹⁴; R¹¹ is hydrogen or C₁₋₈ alkyl; or R¹⁰ and R¹¹ may be taken together with the carbon atom to which they are attached to form a carbonyl group;

R⁵ is hydroxy, esterified hydroxy or etherified hydroxy;

R⁶ is hydrogen, halogen, C₁₋₈ alkyl or C₁₋₈ perfluoroalkyl;

R⁷ is hydrogen, C₁₋₈ alkyl or C₁₋₈ perfluoroalkyl;

R⁸ is —OR¹² or —NR¹²R¹³;

R¹² and R¹³ are each independently hydrogen or C₁₋₈ alkyl;

R¹⁴ is hydrogen, C₁₋₈ alkyl or C₁₋₈ acyl;

X is O, S(O)ₐ, C═O or NR¹⁵;

a is 0, 1 or 2;

R¹⁵ is hydrogen or C₁₋₈ alkyl;

Y and Z for each occurrence are independently (a) hydrogen, (b) halogen, (c) trifluoromethyl, (d) —OCF₃, (e) —CN, (f) C₁₋₈ alkyl optionally substituted with one or more substituents independently selected from the group consisting of halogen, —OCF₃, —CF₃ and phenyl, (g) C₁₋₆ alkoxy, (h) aryl optionally substituted with one or more substituents independently selected from the group consisting of halogen, —OCF₃, —CF₃, C₁₋₄ alkyl and C₁₋₄ alkoxy, (i) —C(O)₂R¹⁶, (j) —C(O)NR¹⁶R¹⁷, (k) —C(O)R¹⁶, (l) —NR¹⁶C(O)NR¹⁶R¹⁷ or (m) —NR¹⁶C(O)R¹⁷; or Y and Z for any occurrence may be taken together to form (a) a carbocycle of the formula —(CH₂)_{b}, or (b) a heterocycle selected from the group consisting of —O(CH₂)_{c}O—, —(CH₂)_{d}NH— and —CH═CHNH—;

b is 3, 4, 5, 6 or 7;

c and d are each independently 2, 3, 4, 5 or 6;

R¹⁶ and R¹⁷ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁸R¹⁹ or C₃₋₁₀ cycloalkyl;

het for each occurrence is a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring is fused to a benzene ring or a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S; and

R¹⁸ and R¹⁹ for each occurrence are independently hydrogen, C₁₋₆ alkyl, C₃₋₁₀ cycloalkyl or aryl optionally substituted with Y and Z.

III. More preferably, the present invention includes the use of the thyromimetic compounds of the following formula, also described in commonly assigned, published European patent application EP 1 088 819:

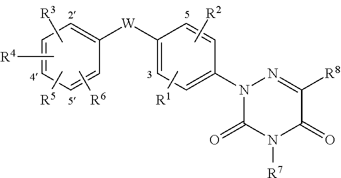

an isomer thereof, a prodrug of said compound or isomer, or a pharmaceutically acceptable salt of said compound, isomer or prodrug; wherein W is (a) —O—, (b) —S(O)ₘ—, (c) —NR³⁰—, (d) —C(O) —, (e) —HC═CH—, (f) —CH₂—, (g) —CHF—, (h) —CF₂— or (i) —CH(OH) —;

R¹ and R² are independently (a) hydrogen, (b) halogen, (c) —(C₁-C₆)alkyl, (d) —CN, (e) —OR¹² or (f) -trifluoromethyl;

R³ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁰, (k) —C(O)R¹⁶, (l) —NR²¹C(O) —NR²¹R²², (m) —NR¹⁹—C(O)R²⁹ or (n) —NR¹⁷R¹⁸;

R⁴ is (a) —C(R¹⁴)(R¹⁵)(R¹⁶), (b) —(C₀-C₃)alkyl-NR¹⁷R¹⁸, (c) —C(O)NR¹⁹R²⁹, (d) —NR¹⁹—C(O) —R²⁹, (e) —(C₀-C₃)alkyl-NR²¹—C(O) —NR²¹R²², (f) —S(O)ₘ—R²², (g) —S(O)₂—NR²¹R²², (h) —NR²¹—S(O)₂—R²², (i) -aryl, (j) -het, (k) —OR³³ or (l) halogen; provided that in substituents (f) and (h), R²² is other than —OR³⁴; and provided that when substituent (b) is —(C₀)alkyl-NR¹⁷R¹⁸, R¹⁸ is other than —C(O) —R²⁸ or —S(O)₂—R²⁹;

or R³ and R⁴ may be taken together to form a carbocyclic ring of Formula —(CH₂)_{b}— or a heterocyclic ring selected from the group consisting of -Q-(CH₂)_{c}— and —(CH₂)ⱼ-Q-(CH₂)ₖ— wherein Q is O, S or NR²⁵; wherein said carbocyclic ring is optionally substituted with one or more substituents independently selected from Group V; and wherein said heterocyclic ring is optionally substituted with one or more substituents independently selected from Group Z;

R⁵ is —OR²³;

or R⁴ and R⁵ may be taken together to form a heterocyclic ring selected from the group consisting of —CR³¹═CR³²—NH—, —N═CR³¹—NH—, —CR³¹═CR³²—O— and —CR³¹═CR³²—S—;

R⁶ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁰, (k) —C(O)R¹⁶, (l) —NR²¹C(O)NR²¹R²², (m) —NR¹⁹—C(O)R²⁰ or (n) —NR¹⁷R¹⁸;

R⁷ is (a) hydrogen, (b) —(C₁-C₄)alkyl wherein each carbon atom is optionally substituted with 1 to 3 halo atoms or (c) —(CH₂)ₙCOOR⁹;

R⁸ is (a) hydrogen, (b) —(C₁-C₆)alkyl, (c) —C(O) —OR⁹, (d) —C(O)NR¹⁰R¹¹ or (e) —CN; provided that in substituent (c), R⁹ is other than methyl or ethyl; and provided that in substitutent (d), R¹⁰ and R¹¹ are not both hydrogen;

R⁹ is (a) —(C₁-C₁₂)alkyl optionally substituted with one or more substitutents independently selected from Group V, (b) —(C₂-C₁₂)alkenyl optionally substituted with phenyl, (c) —(C₂-C₁₂)dialkenyl, (d) —(C₃-C₁₀)cycloalkyl, (e) -aryl or (f) -het;

R¹⁰ and R¹¹ are independently (a) hydrogen, (b) —(C₁-C₁₂)alkyl optionally substituted with one or more substituents independently selected from Group V, (c) —(C₃-C₁₀)cycloalkyl optionally substituted with one or more substituents independently selected from Group V, (d) —(C₂-C₁₂)alkenyl or (e) -het;

or R¹⁰ and R¹¹ for any occurrence may be taken together with the nitrogen atom to which are they attached to form het;

R¹² is (a) hydrogen or (b) —(C₁-C₆)alkyl wherein each carbon atom is optionally substituted with 1 to 3 fluoro atoms;

R¹³ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) —NR¹⁷R¹⁸, (e) -aryl or (f) -het;

R¹⁴ is (a) hydrogen, (b) —(C₁-C₆)alkyl or (c) —O—R³⁴;

R¹⁵ is (a) hydrogen or (b) —(C₁-C₆)alkyl;

or R¹⁴ and R¹⁵ are taken together with the carbon atom to which they are attached to form a carbonyl group;

R¹⁶ is (a) hydrogen, (b) —(C₁-C₆)alkyl wherein each carbon atom is optionally substituted with 1 to 3 fluoro atoms, (c) —(C₀-C₆)alkyl-(C₃-C₁₀)cycloalkyl, (d) —(C₀-C₆)alkyl-aryl or (e) —(C₀-C₆)alkyl-het;

R¹⁷ is (a) hydrogen, (b) —(C₁-C₁₂)alkyl optionally substituted with one or more substituents independently selected from Group V, (c) -aryl, (d) -het, (e) —OR³⁴ or (f) —(C₃-C₁₀)cycloalkyl;

R¹⁸ is (a) hydrogen, (b) —(C₁-C₁₂)alkyl optionally substituted with one or more substituents independently selected from Group V, (c) -aryl, (d) -het, (e) —C(O) —R²⁸, (f) —S(O)₂—R²⁹, (g) —OR³⁴ or (h) —(C₃-C₁₀)cycloalkyl;

or R¹⁷ and R¹⁸ for any occurrence are taken together with the nitrogen atom to which they are attached to form het;

R¹⁹ and R²⁰ for each occurrence are independently (a) hydrogen, (b) —(C₁-C₁₂)alkyl optionally substituted with one or more substituents independently selected from Group V, (c) —(C₀-C₆)alkyl-aryl, (d) —(C₀-C₆)alkyl-het, (e) —C(O) —NR²⁶R²⁷, (f) —C(O) —R²⁸, (g) —S(O)₂—R²⁹, (h) —OR³⁴ or (i) —(C₃-C₁₀)cycloalkyl;

or R¹⁹ and R²⁹ for any occurrence are taken together with the nitrogen atom to which they are attached to form het;

R²¹ and R²² for each occurrence are independently (a) hydrogen, (b) —(C₁-C₁₂)alkyl optionally substituted with one to three substituents independently selected from Group V, (c) -aryl, (d) -het, (e) —(C₃-C₁₀)cycloalkyl or (f) —OR³⁴;
or R²¹ and R²² are taken together with the nitrogen atom to which they are attached to form het;

R²³ is (a) hydrogen, (b) —(C₁-C₄)alkyl optionally substituted with one or more substituents independently selected from Group V or (c) —C(O) —R²⁴;

R²⁴ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;

R²⁵ for each occurrence is independently (a) hydrogen, (b) —(C₁-C₆)alkyl, (c) —COR²⁹ or (d) —SO₂R²⁹;

R²⁸ and R²⁷ for each occurrence are independently (a) hydrogen, (b) —(C₁-C₆)alkyl, (c) —(C₃-C₁₀)cycloalkyl, (d) —(C₀-C₆)alkyl-aryl, or (e) —(C₀-C₆)alkyl-het,

R²⁸ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;

R²⁹ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) -aryl or (e) -het;

R³⁰ is (a) hydrogen, (b) —(C₁-C₁₂)alkyl optionally substituted with one or more substituents independently selected from Group V, (c) —(C₁-C₁₂)alkenyl, (d) —(C₃-C₁₀)cycloalkyl, (e) —C(O) —R³¹ or (f) —S(O), —R³²;

R³¹ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl, (f) -het or (g) —OR³⁴;

R³² is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;

R³³ is (a) —(C₀-C₆)alkyl-aryl, (b) —(C₀-C₆)alkyl-het, (c) —(C₇-C₁₂)alkyl optionally substituted with one or more substituents independently selected from Group V, (d) —(C₁-C₆)alkyl wherein at least one carbon atom is substituted with 1 to 3 fluoro atoms, (e) —(C₂-C₁₂)alkenyl or (f) —(C₃-C₁₀)cycloalkyl;

R³⁴ is (a) -aryl, (b) -het, (c) —(C₁-C₁₂)alkyl optionally substituted with one or more substituents independently selected from Group V, (d) —(C₂-C₁₂)alkenyl or (e)-(C₃-C₁₀)cycloalkyl;

—(C₃-C₁₀)cycloalkyl for each occurrence is a fully or partially saturated mono-, bi- or tricyclic ring containing three to ten carbon atoms; wherein in the bicyclic ring, a monocyclic cycloalkyl ring is spiro used to another cycloalkyl ring or is fused via two carbon atoms to a benzene ring or another cycloalkyl ring; and wherein in the tricyclic ring, a bicyclic ring is spiro fused to a cycloalkyl ring or is fused via two atoms to a benzene ring or another cycloalkyl ring;

said —(C₃-C₁₀)cycloalkyl optionally contains one to three bridging atoms independently selected from carbon, oxygen, sulfur and nitrogen; said bridging atoms are attached to two carbon atoms in the ring; and said bridging atoms are optionally substituted with one to three groups independently selected from —(C₁-C₆)alkyl and hydroxy;

said cycloalkyl ring is optionally substituted on one ring if the moiety is monocyclic, on one or both rings if the moiety is bicyclic, or on one, two or three rings if the moiety is tricyclic, with one or more substitutents independently selected from Group V;

Group V is (a) —(C₁-C₆)alkyl optionally substituted with one or two hydroxy, (b) —(C₂-C₅)alkynyl, (c) -halogen, (d) —NR³⁵R³⁶, (e) —NO₂, (f) —OCF₃, (g) —OR³⁷, (h) —SR³⁷, (i) -oxo, (j) -trifluoromethyl, (k) —CN, (l) —C(O)NR³⁵—OH, (m) —COOR³⁵, (n) —O—C(O) —(C₁-C₆)alkyl, (O) —(C₃-C₁₀)cycloalkyl optionally substituted with CN, (p) —(C₀-C₆)alkyl-aryl, (q) —(C₀-C₆)alkyl-het, (r) —C(O) —(C₁-C₆)alkyl or (s) —C(O) -aryl;

R³⁵ and R³⁶ for each occurrence are independently (a) hydrogen, (b) —(C₁-C₆)alkyl or (c) —(C₀-C₆)alkyl-aryl;

R³⁷ is (a) hydrogen, (b) —(C₁-C₆)alkyl optionally substituted with one or more halo, hydroxy or methoxy, (c) —(C₀-C₆)alkyl-aryl or (d) —(C₀-C₆)alkyl-het;

aryl is (a) phenyl optionally substituted with one or more substituents independently selected from Group Z; (b) naphthyl optionally substituted with one or more substituents independently selected from Group Z or (c) biphenyl optionally substituted with one or more substituents independently selected from Group Z;

het for each occurrence is a 4-, 5-, 6-, 7- and 8-membered fully saturated, partially saturated or fully unsaturated mono-, bi- or tricyclic heterocyclic ring containing from one to four heteroatoms independently selected from the group consisting of oxygen, sulfur and nitrogen; wherein in the bicyclic ring, a monocyclic heterocyclic ring is spiro used to a —(C₃-C₈)cycloalkyl ring or to another heterocyclic ring which is fully or partially saturated; or is fused via two atoms to a benzene ring, a —(C₃-C₈)cycloalkyl ring or another heterocyclic ring; and wherein in the tricyclic ring, a bicyclic ring is spiro fused to a —(C₃-C₈)cycloalkyl ring or to another heterocyclic ring which is fully or partially saturated; or is fused via two atoms to a benzene ring, a (C₃-C₆)cycloalkyl ring, or another heterocyclic ring;

said het optionally contains one to three bridging atoms independently selected from oxygen, sulfur and nitrogen; said bridging atoms are attached to two other atoms in the ring; and said bridging atoms are optionally substituted with one to three groups independently selected from —(C₁-C₆)alkyl and hydroxy;

said het optionally has one or two oxo groups substituted on carbon or one or two oxo groups substituted on sulfur;

said het is optionally substituted on carbon or nitrogen, on one ring if the moiety is monocyclic, on one or both rings if the moiety is bicyclic, or on one, two or three rings if the moiety is tricyclic, with one or more substituents independently selected from Group Z;

Group Z for each occurrence is independently (a) hydrogen, (b) halogen, (c) trifluoromethyl, (d) hydroxy, (e) —OCF₃, (f) —CN, (g) —NO₂, (h) —(C₁-C₆)alkyl optionally substituted with one or more substituents independently selected from the group consisting of hydroxy, halogen, —OCF₃ and —CF₃, (i) —(C₂-C₆)alkenyl optionally substituted with phenyl, (j) —(C₂-C₅)alkynyl, (k) —(C₁-C₆)alkoxy, (l) —(C₀-C₆)alkyl-phenyl optionally substituted with one or more substituents independently selected from the group consisting of halogen, —OCF₃, —CF₃, —(C₁-C₄)alkyl, —(C₁-C₄)alkoxy and —C(O)CH₃, (m) —(C₀-C₆)alkyl-naphthyl optionally substituted with one or more substituents independently selected from the group consisting of halogen, —OCF₃, —CF₃, —(C₁-C₄)alkyl, —(C₁-C₄)alkoxy and —C(O)CH₃, (n) —C(O)₂R³⁵, (O) —(C₀-C₆)alkyl —C(O)NR³⁵R³⁶, (p) —(C₀-C₆)alkyl —C(O)R³⁸, (q) —NR³⁵R³⁶, (r) —NR³⁵—C(O)NR³⁵R³⁶, (s) —NR³⁵—C(O)R³⁶, (t) —OR³⁷, (u) —SR³⁷, (v) —(C₃-C₁₀)cycloalkyl, (w) —(C₀-C₆)alkyl-pyridinyl optionally substituted with one or more —(C₁-C₆)alkyl which is optionally substituted with one or more substituents independently selected from the group consisting of hydroxy and halo, (x) —(C₀-C₆)alkyl-piperidinyl optionally substituted with one or more —(C₁-C₆)alkyl which is optionally substituted with one or more substituents independently selected from hydroxy and halo, (y) —SO₂—R³⁷, (z) —SO₂—NR³⁵R³⁶ or (a1) —S-phenyl-CH₂OH;

R³⁸ is (a) —(C₁-C₆)alkyl, (b) —(C₀-C₆)alkyl-phenyl, (c) —(C₀-C₆)alkyl-phenanthrenyl optionally substituted with one to three CF₃, (d) —(C₀-C₆)alkyl-pyrrolidinyl or (e) —(C₀-C₆)alkyl-morpholinyl;

or any two Z Groups for any occurrence in the same variable may be taken together to form (a) a carbocyclic ring of the formula —(CH₂)ₑ— or (b) a heterocyclic ring selected from the group consisting of —O(CH₂)_{f}O—, —(CH₂)_{g}NH— and —CH═CHNH—;

m is 0, 1 or 2;

n is 0, 1, 2 or 3;

b is 3, 4, 5, 6 or 7;

c, f, g, j and k are each independently 2, 3, 4, 5 or 6; and

e is 3, 4, 5, 6 or 7;

provided that in a compound of the above formula: 1) the substituent —C(R¹⁴)(R¹⁵)(R¹⁶) in R⁴ is other than (C₁-C₄)alkyl; and 2) R⁴ is halo only when R⁸ is —C(O) —OR⁹ or —C(O)NR¹⁰R¹¹.

More particularly, the following compounds are useful in the methods of the present invention:
8-[[5-[2,6-dichloro-4-(4,5-dihydro-3,5-dioxo-1,2,4-triazine-2(3H)-yl)phenoxy]-2-hydroxyphenyl]sulfonyl]-spiro[8-azabicyclo[3.2.1]octane-3,2′-(3′H)-dihydro-furan];
2-{3,5-dichloro-4-[3-(3,3-dimethyl-piperidine-1-sulfonyl)-4-hydroxy-phenoxy]-phenyl}-2H-[1,2,4]triazine-3,5-dione;
2-{3,5-dichloro-4-[4-hydroxy-3-(3-methyl-3-phenyl-piperidine-1-sulfonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzenesulfonamide;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,3-dimethyl-piperidine-1-carbonyl)-4-hydroxy-phenoxy]-phenyl}-2H-[1,2,4]triazine-3,5-dione;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[4-hydroxy-3-(3-methyl-3-phenyl-piperidine-1-carbonyl)-phenoxy]-phenyl}-2H-[1,2,4]triazine-3,5-dione;
5-[2,6-dichloro-4-(3,5-dioxo-4,5-dihydro-3H-[1,2,4]triazin-2-yl)-phenoxy]-N-(6,6-dimethyl-bicyclo[3.1.1]hept-2-yl)-2-hydroxy-benzamide;
2-{3,5-dichloro-4-[3-(3,5-dimethyl-piperidine-1-carbonyl)-4-hydroxy-phenoxy]-phenyl}-2H-[1,2,4]triazine-3,5-dione;
2-{3,5-dichloro-4-[4-hydroxy-3-(piperidine-1-carbonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,4-dihydro-1H-isoquinoline-2-carbonyl)-4-hydroxy-phenoxy]-phenyl}-2H-[1,2,4]triazine-3,5-dione;
2-{4-[3-(4-fluoro-benzyl)-4-hydroxy-phenoxy]-3,5-dimethyl-phenyl}-2H-[1,2,4]triazine-3,5-dione; and
2-{3,5-dichloro-4-[3-(4-fluoro-benzoyl)-4-hydroxy-phenoxy]-phenyl}-2H-[1,2,4]triazine-3,5-dione.

IV. Also, the thyromimetic compounds useful in the methods of the present invention have the following formula, also described in commonly assigned published European Patent Application 1 127 882:

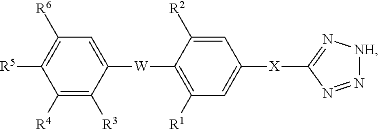

or a stereoisomer, pharmaceutically acceptable salt or prodrug thereof, or a pharmaceutically acceptable salt of the prodrug, wherein:

W is O, S, SO, SO₂, CH₂, CF₂, CHF, C(═O), CH(OH), NR^{a}, or

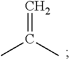

X is O, CH₂, CH₂CH₂, S, SO, SO₂, CH₂NR^{a}, NR^{a}, or a bond;

each R^{a} is independently hydrogen, C₁-C₆alkyl, or C₁-C₆alkyl substituted with one substituent selected from C₃-C₆cycloalkyl or methoxy;

R¹, R², R³ and R⁶ are independently hydrogen, halogen, C₁-C₈alkyl, —CF₃, —OCF₃, —OC₁-C₈alkyl, or —CN;

R⁴ is hydrogen, C₁-C₁₂alkyl, [C₁-C₁₂alkyl that is substituted with from one to three substituents independently selected from Group V], C₂-C₁₂ alkenyl, C₂-C₁₂ alkynyl, halogen, —CN, —OR^{b}, —SR^{c}, —S(═O)R^{c}, —S(═O)₂R^{c}, aryl, heteroaryl, C₃-C₁₀ cycloalkyl, heterocycloalkyl, —S(═O)₂NR^{c}R^{d}, —C(═O)NR^{c}R^{d}, —C(═O)OR^{c}, —NR^{a}C(═O)R^{d}, —NR^{a}C(═O)NR^{c}R^{d}, —NR^{a}S(═O)₂R^{d}, —NR^{a}R^{d}, —C(═O)R^{c},

or R³ and R⁴ may be taken together with the carbon atoms to which they are attached to form an unsubstituted or substituted carbocyclic ring of formula —(CH₂)ᵢ— or an unsubstituted or substituted heterocyclic ring selected from the group consisting of -Q-(CH₂)ⱼ— and —(CH₂)ₖ-Q-(CH₂)ₗ— wherein Q is O, S or NR^{a}; i is 3, 4, 5, 6 or 7; j is 2, 3, 4, 5, or 6; k and l are each independently 1, 2, 3, 4, or 5, and any substituents up to four are selected from C₁-C₄alkyl, —OR^{b}, oxo, —CN, phenyl, or —NR^{a}R^{g};

R^{b} is hydrogen, C₁-C₁₂alkyl, [C₁-C₁₂alkyl substituted with one to three substituents independently selected from Group V], aryl, heteroaryl, C₃-C₁₀ cycloalkyl, heterocycloalkyl, —C(═O)NR^{c}R^{d}, or —C(═O)R^{f};

R^{c} and R^{d} are each independently selected from hydrogen, C₁-C₁₂alkyl, [C₁-C₁₂alkyl substituted with one to three substituents independently selected from Group VI], C₂-C₁₂alkenyl, C₂-C₁₂alkynyl, aryl, heteroaryl, C₃-C₁₀ cycloalkyl, heterocycloalkyl,

or R^{c} and R^{d} may together along with the atom(s) to which they are attached form a 3-10 membered unsubstituted or substituted heterocyclic ring, which may contain a second heterogroup selected from O, NR^{e}, or S, wherein any substitutents up to four are selected from C₁-C₄alkyl, —OR^{b}, oxo, —CN, phenyl, or —NR^{a}R^{g};

R⁵ is —OH, —OC₁-C₆alkyl, —OC(═O)R^{f}, —F, —C(═O)OR^{c},

or R⁴ and R⁵ may together with the atom(s) to which they are attached form a heterocyclic ring selected from the group consisting of —CR^{c}═CR^{a}—NH—, —N═CR^{a}—NH—, —CR^{c}═CR^{a}—O—, —CR^{c}═CR^{a}—S—, —CR^{c}═N—NH—, or —CR^{a}═CR^{a}—CR^{a}═N—;

Group V is halogen, —CF₃, —OCF₃, hydroxy, oxo, C₁-C₆alkoxy, —CN, aryl, heteroaryl, C₃-C₁₀cycloalkyl, heterocycloalkyl, —SR^{f}, —S(═O)R^{f}, —S(═O)₂R^{f}, [—S(═O)₂NR^{a}R^{f}, wherein R^{a} and R^{f} may together along with the atom(s) to which they are attached form a 3-8 membered heterocyclic ring, which may contain a second heterogroup selected from O, NR^{e} or S], —NR^{a}R^{g}, or [—C(═O)NR^{a}R^{f}, wherein R^{a} and R^{f} may together along with the atom(s) to which they are attached form a 3-8 membered heterocyclic ring, which may contain a second heterogroup selected from O, NR^{e} or S];

Group VI is halogen, hydroxy, oxo, C₁-C₆alkoxy, aryl, heteroaryl, C₃-C₈cycloalkyl, heterocycloalkyl, —CN, or —OCF₃;

R^{e} is hydrogen, —CN, C₁-C₁₀alkyl, [C₁-C₁₀alkyl substituted with one to three substitutents independently selected from Group V], C₂-C₁₀alkenyl, C₂-C₁₀alkoxy, C₃-C₁₀cycloalkyl, aryl, heteroaryl, —C(═O)R^{f}, —C(═O)OR^{f}, —C(═O)NR^{a}R^{f}, —S(═O)₂NR^{e}R^{f}, or

—S(═O)₂R^{f};

R^{f} is hydrogen, C₁-C₁₀alkyl, [C₁-C₁₀alkyl substituted with from one to three substituents selected from Group VI], C₂-C₁₀alkenyl, C₂-C₁₀alkoxy, C₃-C₁₀cycloalkyl, heterocycloalkyl, aryl, or heteroaryl; and

R⁹ is hydrogen, C₁-C₆alkyl, C₃-C₈cycloalkyl, C₂-C₆ alkenyl, aryl, —C(═O)R^{f}, —C(═O)OR^{f}, —C(═O)NR^{a}R^{f}, or —S(═O)₂R^{f}, provided that R¹ and R² are not both hydrogen, further provided that when X is CH₂, W is NR^{a}, R³ is hydrogen and R⁵ is —OH, then R⁶ and R⁴ are not both —C(CH₃)₃, further provided that when X is CH₂ or CH₂CH₂, W is O, and R³ and R⁶ are hydrogen, then R⁴ is not halogen, —CF₃, C₁-C₆alkyl or C₃-C₇cycloalkyl, and further provided that when R³ and R⁴ are hydrogen and W is O then R⁶ is not halogen, —CF₃, C₁-C₆alkyl or C₃-C₇cycloalkyl.

V. In another embodiment, the present invention includes the use of the thyromimetic compounds of the following formula, also described in commonly assigned, published European Patent Application 1 148 054:

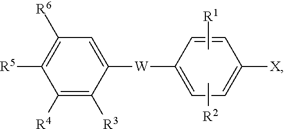

the stereoisomer and prodrug thereof, and the pharmaceutically acceptable salt of said compound, stereoisomer, and prodrug, wherein:

W is oxygen, sulfur, —SO—, —S(O)₂, —CH₂—, —CF₂—, —CHF—, —C(O) —, —CH(OH) —, —NR^{a}, or —C(═CH₂) —;

R¹, R², R³, and R⁶ are each independently hydrogen, halogen, —(C₁-C₈)alkyl, —CF₃, —OCF₃, —O(C₁-C₈)alkyl, or —CN;

R⁴ is hydrogen, —(C₁-C₁₂)alkyl substituted with zero to three substituents independently selected from Group V, —(C₂-C₁₂)alkenyl, —(C₂-C₁₂)alkynyl, halogen, —CN, —OR^{b}, —SR^{c}, —S(O)R^{c}, —S(O)₂R^{c}, aryl, heteroaryl, —(C₃-C₁₀)cycloalkyl, heterocycloalkyl, —S(O)₂NR^{c}R^{d}, —C(O)NR^{c}R^{d}, —C(O)OR^{c}, —NR^{a}C(O)R^{d}, —NR^{a}C(O)NR^{c}R^{d}, —NR^{a}S(O)₂R^{d}, or —C(O)R^{c}; or

R³ and R⁴ are taken together along with the carbon atoms to which they are attached to form a carbocyclic ring of formula —(CH₂)ᵢ— or a heterocyclic ring of formula —(CH₂)ₖ-Q-(CH₂)ₗ— wherein Q is oxygen, sulfur, or —NR^{e}—; i is 3, 4, 5, or 6; k is 0, 1, 2, 3, 4, or 5; and l is 0, 1, 2, 3, 4, or 5; and wherein said carbocyclic ring and said heterocyclic ring are each substituted with zero to four substituents independently selected from —(C₁-C₄)alkyl, —OR^{b}, oxo, —CN, phenyl, or —NR^{a}R^{g};

R⁵ is hydroxy, —O(C₁-C₆)alkyl, —OC(O)R^{f}, fluorine, or —C(O)OR^{c}; or

R⁴ and R⁵ are taken together along with the carbon atoms to which they are attached to form a heterocyclic ring selected from the group consisting of —CR^{c}═CR^{a}—NH—, —N═CR^{a}—NH, —CR^{c}═CR^{a}—O—, —CR^{c}═CR^{a}—S—, —CR^{c}═N—NH—, and —CR^{a}═CR^{a}—CR^{a}═N—;

R^{a} for each occurrence is independently hydrogen, or —(C₁-C₆)alkyl substituted with zero or one —(C₃-C₆)cycloalkyl or methoxy;

R^{b} for each occurrence is independently hydrogen, —(C₁-C₁₂)alkyl substituted with zero to three substituents independently selected from Group V, aryl, heteroaryl, —(C₃-C₁₀)cycloalkyl, heterocycloalkyl, —C(O)NR^{c}R^{d}, or —C(O)R^{f};

R^{c} and R^{d} for each occurrence are each independently hydrogen, —(C₁-C₁₂)alkyl substituted with zero to three substituents independently selected from Group VI, —(C₂-C₁₂)alkenyl, —(C₂-C₁₂)alkynyl, aryl, heteroaryl, —(C₃-C₁₀)cycloalkyl, or heterocycloalkyl;

provided that when R⁴ is the moiety —SR^{c}, —S(O)R^{c}, or —S(O)₂R^{c}, R^{c} is other than hydrogen; or

R^{c} and R^{d} are taken together along with the atom(s) to which they are attached to form a 3-10 membered heterocylic ring which may optionally contain a second heterogroup selected from oxygen, —NR^{e}—, or sulfur; and wherein said heterocyclic ring is substituted with zero to four substituents independently selected from —(C₁-C₄)alkyl, —OR^{b}, oxo, —CN, phenyl, or —NR^{a}R^{g};

R^{e} for each occurrence is hydrogen, —CN, —(C₁-C₁₀)alkyl substituted with zero to three substituents independently selected from Group V, —(C₂-C₁₀)alkenyl, —(C₂-C₁₀)alkoxy, —(C₃-C₁₀)cycloalkyl, aryl, heteroaryl, —C(O)R^{f}, —C(O)OR^{f}, —C(O)NR^{a}R^{f}, or —S(O)₂R^{f};

R^{f} for each occurrence is independently —(C₁-C₁₀)alkyl substituted with zero to three substituents independently selected from Group VI, —(C₂-C₁₂)alkenyl, —(C₂-C₁₀)alkynyl, —(C₃-C₁₀)cycloalkyl, aryl, heteroaryl, or heterocycloalkyl;

R^{g} for each occurrence is independently hydrogen, —(C₁-C₆)alkyl, —(C₂-C₆)alkenyl, aryl, —C(O)R^{f}, —C(O)OR^{f}, —C(O)NR^{a}R^{f}, —S(O)₂R^{f}, or —(C₃-C₈)cycloalkyl;

Group V is halogen, —CF₃, —OCF₃, —OH, oxo, —(C₁-C₆)alkoxy, —CN, aryl, heteroaryl, —(C₃-C₁₀)cycloalkyl, heterocycloalkyl, —SR^{f}, —S(O)R^{f}, —S(O)₂R^{f}, —S(O)₂NR^{a}R^{f}, —NR^{a}R^{g}, or —C(O)NR^{a}R^{f};

Group VI is halogen, hydroxy, oxo, —(C₁-C₆)alkoxy, aryl, heteroaryl, —(C₃-C₈)cycloalkyl, heterocycloalkyl, —CN, or —OCF₃;

provided that when R⁴ is —(C₁-C₁₂)alkyl substituted with zero to three substituents independently selected from Group V, wherein said Group V substituent is oxo, said oxo group is substituted on a carbon atom other than the C₁ carbon atom in —(C₁-C₁₂)alkyl;

aryl for each occurrence is independently phenyl or naphthyl substituted with zero to four substituents independently selected from halogen, —(C₁-C₆)alkyl, —CN, —SR^{f}, —S(O)R^{f}, —S(O)₂R^{f}, —(C₃-C₆)cycloalkyl, —S(O)₂NR^{a}R^{f}, —NR^{a}R^{g}, —C(O)NR^{a}R^{f}, —OR^{b}, -perfluoro-(C₁-C₄)alkyl, or —COOR^{f};

provided that when said substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, said substituents R^{b}, R^{f}, and R^{g}, are other than aryl or heteroaryl;

heteroaryl for each occurrence is independently a 5-, 6-, 7-, 8-, or 9-membered monocyclic or bicyclic ring having from one to three heteroatoms selected from O, N, or S;

wherein in said bicyclic ring, a monocyclic heteroaryl ring is fused to a benzene ring or to another heteroaryl ring, and having zero to three substituents independently selected from halogen, —(C₁-C₄)alkyl, —CF₃, —OR^{b}, —NR^{a}R^{g}, or —COOR^{f};

provided that when said substituent(s) on heteroaryl are —NR^{a}R^{g}, —OR^{b}, or —COOR^{f}, said substituents R^{b}, R^{f}, and R^{g}, are other than aryl or heteroaryl; heterocycloalkyl for each occurrence is independently a 5-, 6-, 7-, 8-, or 9-membered monocyclic or bicyclic cycloalkyl ring having from one to three heteroatoms selected from oxygen, —NR^{e}, or sulfur, and having zero to four substituents independently selected from —(C₁-C₄)alkyl, —OR^{b}, oxo, —CN, phenyl, or —NR^{a}R^{g}; and

X is

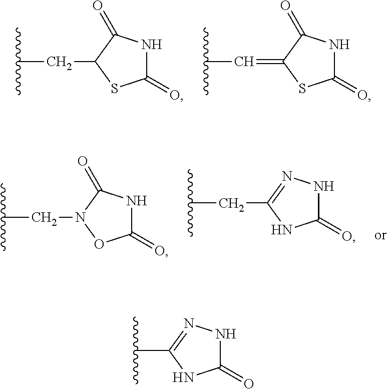

VI. Also, the thyromimetic compounds useful in the methods of the present invention have the following formula, also described in commonly assigned, published International patent application WO 01/72692:

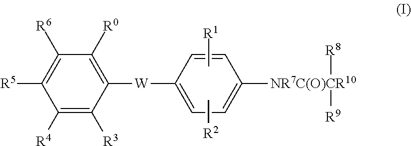

an isomer thereof, a prodrug of said compound or isomer, or a pharmaceutically acceptable salt of said compound, isomer or prodrug; wherein W is (a) —O—, (b) —S—, (c) —SO—, (d) —SO₂—, (e) —CH₂—, (f) —CF₂—, (g) —CHF—, (h) —C(O) —, (i) —CH(OH) —, (j) —NR^{a} or (k)

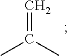

R⁰ is (a) hydrogen, (b) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (c) —C(O)R^{h}, (d) —S(O)₂R^{h} or (e) halogen;

R¹, R², R³ and R⁶ are each independently (a) hydrogen, (b) halogen, (c) —(C₁-C₈)alkyl, (d) —CF₃, (e) —OCF₃, (f) —O(C₁-C₈)alkyl, or (g) —CN;

R⁴ is (a) hydrogen, (b) —(C₁-C₁₂)alkyl substituted with zero to three substituents independently selected from Group V, (c) —(C₂-C₁₂)alkenyl, (d) —(C₂-C₁₂)alkynyl, (e) halogen, (f) —CN, (g) —OR^{b}, (h) —SR^{c}, (i) —S(O)R^{c}, (j) —S(O)₂R^{c}, (k) aryl, (l) heteroaryl, (m) —(C₃-C₁₀)cycloalkyl, (n) heterocycloalkyl, (O) —S(O)₂NR^{c}R^{d}, (p) —C(O)NR^{c}R^{d}, (q) —C(O)OR^{C}, (r) —NR^{a}C(O)R^{d}, (s) —NR^{a}C(O)NR^{c}R^{d}, (t) —NR^{a}S(O)₂R^{d}, (u) —NR^{a}R^{d} or (v) —C(O)R^{c};

or R³ and R⁴ are taken together along with the carbon atoms to which they are attached to form a carbocyclic ring of formula —(CH₂)ᵢ— or a heterocyclic ring of formula —(CH₂)ₖ-Q-(CH₂)ₗ— wherein Q is —O—, —S— or —NR^{e}—; i is 3, 4, 5 or 6; k is 0, 1, 2, 3, 4 or 5; and l is 0, 1, 2, 3, 4 or 5; and wherein the carbocyclic ring and the heterocyclic ring are each substituted with zero to four substituents independently selected from (a) —(C₁-C₄)alkyl, (b) —OR^{b}, (c) oxo, (d) —CN, (e) phenyl or (f) —NR^{a}R^{g};

R⁵ is (a) —OH, (b) —O(C₁-C₆)alkyl, (c) —OC(O)R^{f}, (d) F, or (e) —C(O)OR^{c};

or R⁴ and R⁹ are taken together along with the carbon atoms to which they are attached to form a heterocyclic ring selected from the group consisting of —CR^{c}═CR^{a}—NH—, —N═CR^{a}—NH, —CR^{c}═CR^{a}—O—, —CR^{c}═CR^{a}—S—, —CR^{c}═N—NH— and —CR^{a}═CR^{a}—CR^{a}═N—;

R⁷ is (a) hydrogen or (b) —(C₁-C₆)alkyl;

R⁸ and R⁹ are each independently (a) hydrogen, (b) —(C₁-C₆)alkyl, (c) aryl, or (d) halogen;

R¹⁰ is (a) —(C₀-C₁)alkyl-C(O)OH, (b) —(C₀-C₁)alkyl-C(O)OR^{f}, (c) —(C₀-C₁)alkyl-C(O)NR^{c}R^{d}, or (d) —(C₀-C₁)alkyl-OH;

R^{a} for each occurrence is independently (a) hydrogen or (b) —(C₁-C₆)alkyl substituted with zero or one —(C₃-C₆)cycloalkyl or methoxy;

R^{b} for each occurrence is independently (a) hydrogen, (b) —(C₁-C₁₂)alkyl substituted with zero to three substituents independently selected from Group V, (c) aryl, (d) heteroaryl, (e) —(C₃-C₁₀)cycloalkyl, (f) heterocycloalkyl, (g) —C(O)NR^{c}R^{d}, or (h) —C(O)R^{f};

R^{c} and R^{d} for each occurrence are each independently (a) hydrogen, (b) —(C₁-C₁₂)alkyl substituted with zero to three substituents independently selected from Group VI, (c) —(C₂-C₁₂)alkenyl, (d) —(C₂-C₁₂)alkynyl, (e) aryl, (f) heteroaryl, (g) —(C₃-C₁₀)cycloalkyl or (h) heterocycloalkyl;

provided that when R⁴ is the moiety —SR^{c}, —S(O)R^{c} or —S(O)₂R^{c}, R^{c} is other than hydrogen;

or R^{c} and R^{d} are taken together along with the atom(s) to which they are attached to form a 3-10 membered heterocyclic ring which may optionally contain a second heterogroup selected from —O—, —NR^{e}— or —S—; and wherein the heterocyclic ring is substituted with zero to four substituents independently selected from (a) —(C₁-C₄)alkyl, (b) —OR^{b}, (c) oxo, (d) —CN, (e) phenyl or (f) —NR^{a}R^{g};

R^{e} for each occurrence is (a) hydrogen, (b) —CN, (c) —(C₁-C₁₀)alkyl substituted with zero to three substitutents independently selected from Group V, (d) —(C₂-C₁₀)alkenyl, (e) —(C₂-C₁₀)alkoxy, (f) —(C₃-C₁₀)cycloalkyl, (g) aryl, (h) heteroaryl, (i) —C(O)R^{f}, (j) —C(O)OR^{f}, (k) —C(O)NR^{a}R^{f} or (l) —S(O)₂R^{f};

R^{f} for each occurrence is independently (a) —(C₁-C₁₀)alkyl substituted with zero to three substituents independently selected from the Group VI, (b) —(C₂-C₁₀)alkenyl, (c) —(C₂-C₁₀)alkynyl, (d) —(C₃-C₁₀)cycloalkyl, (e) aryl, (f) heteroaryl or (g) heterocycloalkyl;

R^{g} for each occurrence is independently (a) hydrogen, (b) —(C₁-C₆)alkyl, (c) —(C₂-C₆)alkenyl, (d) aryl, (e) —C(O)R^{f}, (f) —C(O)OR^{f}, (g) —C(O)NR^{a}R^{f}, (h) —S(O)₂R^{f} or (i) —(C₃-C₈)cycloalkyl;

R^{h} is (a) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (b) phenyl substituted with zero to two substituents independently selected from the group consisting of (1) —(C₁-C₄)alkyl, (2) halogen, (3) —CF₃ and (4) —OCF₃; (c) —(C₃-C₆)cycloalkyl or (d) heterocycloalkyl;

Group V is (a) halogen, (b) —CF₃, (c) —OCF₃, (d) —OH, (e) -oxo, (f) —(C₁-C₆)alkoxy, (g) —CN, (h) aryl, (i) heteroaryl, (j) —(C₃-C₁₀)cycloalkyl, (k) heterocycloalkyl, (l) —SR^{f}, (m) —S(O)R^{f}, (n) —S(O)₂R^{f}, (O) —S(O)₂NR^{a}R^{f} (p) —NR^{a}R^{g} or (q) —C(O)NR^{a}R^{f};

Group VI is (a) halogen, (b) hydroxy, (c) oxo, (d) —(C₁-C₆)alkoxy, (e) aryl, (f) heteroaryl, (g) —(C₃-C₆)cycloalkyl, (h) heterocycloalkyl, (i) —CN, or (j) —OCF₃;

provided that when the substituent R⁴ is —(C₁-C₁₂)alkyl substituted with zero to three substituents independently selected from Group V wherein the Group V substituent is oxo, the oxo group is substituted on a carbon atom other than the C₁ carbon atom in —(C₁-C₁₂)alkyl;

aryl for each occurrence is independently phenyl or naphthyl substituted with zero to four substituents independently selected from (a) halogen, (b) —(C₁-C₆)alkyl, (c) —CN, (d) —SR^{f}, (e) —S(O)R^{f}, (f) —S(O)₂R^{f}, (g) —(C₃-C₆)cycloalkyl, (h) —S(O)₂NR^{a}R^{f}, (i) —NR^{a}R^{g}, (j) —C(O)NR^{a}R^{f}, (k) —OR^{b}, (l) -perfluoro-(C₁-C₄)alkyl, or (m) —COOR^{f};

provided that when the substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, the substituents R^{b}, R^{f} and R^{g} are other than aryl or heteroaryl;

heteroaryl for each occurrence is independently a 5-, 6-, 7-, 8-, 9- or 10-membered monocyclic or bicyclic ring having from 1 to 3 heteroatoms selected from O, N or S; wherein in the bicyclic ring, a monocyclic heteroaryl ring is fused to a benzene ring or to another heteroaryl ring; and having zero to three substituents independently selected from (a) halogen, (b) —(C₁-C₄)alkyl, (c) —CF₃, (d) —OR^{b}, (e) —NR^{a}R^{g}, or (f) —CO₂R^{f};

provided that when the substituent(s) on heteroaryl are —OR^{b}, —NR^{a}R^{g} or —CO₂R^{f}, the substituents R^{b}, R^{f} and R^{g} are other than aryl or heteroaryl;

heterocycloalkyl for each occurrence is independently a 4-, 5-, 6-, 7-, 8-, 9- or 10-membered monocyclic or bicyclic cycloalkyl ring having from 1 to 3 heteroatoms selected from O, NR^{e} or S; and having zero to four substituents independently selected from (a) —(C₁-C₄)alkyl, (b) —OR^{b}, (c) oxo, (d) —CN, (e) phenyl or (f) —NR^{a}R^{g}.

More particularly, the following compounds are useful in the methods of the present invention:
N-{4-[3-(cyclobutyl-methyl-carbamoyl)-4-hydroxy-phenoxy]-3,5-dimethyl-phenyl}-malonamic acid;

N-{3-chloro-4-[4-hydroxy-3-(1-isopropyl-2-methyl-propylcarbamoyl)-phenoxy]-5-methyl-phenyl}-malonamic acid;
N-{3,5-dichloro-4-[3-((1S)-cyclohexyl-ethylcarbamoyl)-4-hydroxy-phenoxy]-phenyl}-malonamic acid;
N-[3,5-dichloro-4-(3-cyclopropylsulfamoyl-4-hydroxy-phenoxy)-phenyl]-malonamic acid;
N-[3,5-dichloro-4-(3-cyclobutylsulfamoyl-4-hydroxy-phenoxy)-phenyl]-malonamic acid;
N-[3-chloro-4-(3-cyclobutylsulfamoyl-4-hydroxy-phenoxy)-5-methyl-phenyl]-malonamic acid;
N-[4-(3-cyclobutylsulfamoyl-4-hydroxy-phenoxy)-3,5-dimethyl-phenyl]-malonamic acid;
N-[4-(3-cyclopropylsulfamoyl-4-hydroxy-phenoxy)-3,5-dimethyl-phenyl]-malonamic acid;
N-[3-chloro-4-(3-cyclobutylmethanesulfonyl-4-hydroxy-phenoxy)-5-methyl-phenyl]-malonamic acid;
N-[3-chloro-4-(3-cyclopropylmethanesulfonyl-4-hydroxy-phenoxy)-5-methyl-phenyl]-malonamic acid;
N-[3,5-dichloro-4-(3-cyclopropylmethanesulfonyl-4-hydroxy-phenoxy)-phenyl]-malonamic acid;
N-[4-(3-cyclobutylmethanesulfonyl-4-hydroxy-phenoxy)-3,5-dimethyl-phenyl]-malonamic acid;
N-[3,5-dichloro-4-(3-cyclobutylmethanesulfonyl-4-hydroxy-phenoxy)-phenyl]-malonamic acid;
N-[4-(3-cyclopentylmethanesulfonyl-4-hydroxy-phenoxy)-3,5-dimethyl-phenyl]-malonamic acid;
N-[4-(3-cyclobutylmethanesulfonyl-4-hydroxy-phenoxy)-3,5-dimethyl-phenyl]-2-methyl-malonamic acid;
N-[3-chloro-4-(3-cyclohexylmethanesulfonyl-4-hydroxy-phenoxy)-5-methyl-phenyl]-malonamic acid;
N-[3-chloro-4-(3-cyclobutylmethanesulfonyl-4-hydroxy-phenoxy)-5-methyl-phenyl]-2-methyl-malonamic acid;
N-[3-chloro-4-(3-cyclopentylmethanesulfonyl-4-hydroxy-phenoxy)-5-methyl-phenyl]-malonamic acid;
N-[4-(3-cyclohexylmethanesulfonyl-4-hydroxy-phenoxy)-3,5-dimethyl-phenyl]-malonamic acid;
N-{4-[3-(4-fluoro-benzenesulfonyl)-4-hydroxy-phenoxy]-3,5-dimethyl-phenyl}-malonamic acid;
N-{3-chloro-4-[3-(4-fluoro-benzenesulfonyl)-4-hydroxy-phenoxy]-5-methyl-phenyl}-malonamic acid;
N-{4-[3-(4-fluoro-benzoyl)-4-hydroxy-phenoxy]-3,5-dimethyl-phenyl}-malonamic acid;
N-[4-(3-cyclopentylacetyl-4-hydroxy-phenoxy)-3,5-dimethyl-phenyl]-malonamic acid;
N-(4-{3-[(4-fluoro-phenyl)-hydroxy-methyl]-4-hydroxy-phenoxy}-3,5-dimethyl-phenyl)-malonamic acid;
N-{-4-[3-(2-cyclopentyl-1-hydroxy-ethyl)-4-hydroxy-phenoxy]-3,5-dimethyl-phenyl}-malonamic acid;
N-[3-chloro-4-(3-cyclobutylmethanesulfonyl-4-hydroxy-phenoxy)-5-methyl-phenyl]-malonamic acid methyl ester;
N-[3-chloro-4-(3-cyclobutylmethanesulfonyl-4-hydroxy-phenoxy)-5-methyl-phenyl]-malonamic acid ethyl ester;
N-[4-(3-cyclobutylmethanesulfonyl-4-hydroxy-phenoxy)-3,5-dimethyl-phenyl]-malonamic acid ethyl ester;
N-[4-(3-cyclobutylmethanesulfonyl-4-hydroxy-phenoxy)-3,5-dimethyl-phenyl]-malonamic acid methyl ester;
N-[3-chloro-4-(3-cyclopentanesulfonyl-4-hydroxy-phenoxy)-5-methyl-phenyl]-malonamic acid;
N-[4-(3-cyclopentanesulfonyl-4-hydroxy-phenoxy)-3,5-dimethyl-phenyl]-malonamic acid;
N-{3-chloro-4-[3-(4-fluoro-benzenesulfonyl)-4-hydroxy-phenoxy]-5-methyl-phenyl}-malonamic acid;
N-{3-chloro-4-[3-(4-fluoro-benzenesulfonyl)-4-hydroxy-phenoxy]-5-methyl-phenyl}-malonamic acid methyl ester;
N-{3,5-dichloro-4-[3-(4-fluoro-benzenesulfonyl)-4-hydroxy-phenoxy]-phenyl}-malonamic acid methyl ester;
N-{3,5-dichloro-4-[3-(4-fluoro-benzenesulfonyl)-4-hydroxy-phenoxy]-phenyl}-malonamic acid ethyl ester;
N-{4-[3-(4-fluoro-benzenesulfonyl)-4-hydroxy-phenoxy]-3,5-dimethyl-phenyl}-2-methyl-malonamic acid methyl ester;
N-{4-[3-(4-fluoro-benzenesulfonyl)-4-hydroxy-phenoxy]-3,5-dimethyl-phenyl}-2-methyl-malonamic acid;
N-{3-chloro-4-[3-(4-fluoro-benzenesulfonyl)-4-hydroxy-phenoxy]-5-methyl-phenyl}-2-methyl-malonamic acid methyl ester;
N-{3-chloro-4-[3-(4-fluoro-benzenesulfonyl)-4-hydroxy-phenoxy]-5-methyl-phenyl}-2-methyl-malonamic acid;
N-{3,5-dichloro-4-[3-(4-fluoro-benzenesulfonyl)-4-hydroxy-phenoxy]-phenyl}-2-methyl-malonamic acid methyl ester; and
N-{3,5-dichloro-4-[3-(4-fluoro-benzenesulfonyl)-4-hydroxy-phenoxy]-phenyl}-2-methyl-malonamic acid;
N-{4-[3-(4-fluoro-benzenesulfonyl)-4-hydroxy-phenoxy]-3,5-dimethyl-phenyl}-malonamic acid; and
N-{4-[3-(4-fluoro-benzenesulfonyl)-4-hydroxy-phenoxy]-3,5-dimethyl-phenyl}-malonamic acid methyl ester; or an isomer thereof, a prodrug of said compound or isomer, or a pharmaceutically acceptable salt of said compound, isomer or prodrug.

VII. In another embodiment, the present invention includes the use of the thyromimetic compounds of the following formula, also described in commonly assigned, U.S. patent application Ser. No. 10/255,180, filed Sep. 24, 2002,

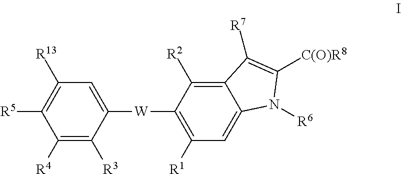

or the pharmaceutically acceptable salt thereof; wherein

W is oxygen, CH₂, CF₂, NR¹², S(O)ₘ wherein m is 0, 1 or 2;

R¹, R², and R³ are each independently selected from the group consisting of hydrogen, halo, cyano, trifluoromethyl, trifluomethoxy and (C₁-C₆)alkyl;

R⁴ is hydrogen, halo, cyano, (C₁-C₁₂)alkyl, (C₂-C₁₂)alkenyl, (C₂-C₁₂)alkynyl, (C₃-C₁₀)cycloalkyl, (C₃-C₁₀)cycloalkyl(C₁-C₆)alkyl, (C₆-C₁₀)aryl, (C₆-C₁₀)aryl(C₁-C₆)alkyl, (C₂-C₉)heteroaryl, (C₂-C₉)heteroaryl(C₁-C₆)alkyl, (C₂-C₉)heterocycloalkyl, (C₂-C₉)heterocycloalkyl(C₁-C₆)alkyl, —OR⁹, —S(O)₂NR¹⁰R¹¹, —C(O)NR¹⁰R¹¹, —C(O)R¹⁰, —CH(OH)R¹⁰, —NR¹²C(O)R¹⁹, —NR¹²C(O)NR¹⁰R¹¹, —NR¹²S(O)₂R¹⁰ or —S(O)₂R¹⁰ wherein n is 0, 1 or 2;

R⁵ is hydroxy, fluoro, (C₁-C₄)alkoxy or —OC(O)R¹⁰;

R⁶ is hydrogen, —C(O)CH₃ or (C₁-C₆)alkyl;

R⁷ is hydrogen or (C₁-C₆)alkyl;

R⁸ is OR¹² or NR⁹R¹²;

R⁹ for each occurrence is independently hydrogen, (C₁-C₁₂)alkyl, (C₃-C₁₀)cycloalkyl, (C₂-C₉)heterocycloalkyl, (C₆-C₁₀)aryl or (C₂-C₉)heteroaryl;

R¹⁰ for each occurrence is independently hydrogen, (C₁-C₁₂)alkyl, (C₂-C₁₂)alkenyl, (C₂-C₁₂)alkynyl, (C₃-C₁₀)cycloalkyl, (C₃-C₉)cycloalkyl(C₁-C₆)alkyl, (C₆-C₁₀)aryl, (C₂-C₉)heteroaryl or halo(C₆-C₁₀)aryl;

R¹¹ for each occurrence is independently hydrogen, (C₁-C₆)alkyl, (C₃-C₁₀) cycloalkyl or (C₃-C₉)cycloalkyl(C₁-C₆)alkyl;

or R¹⁰ and R¹¹ may be taken together with the nitrogen to which they are attached to form a 3 to 10 membered heterocyclic group which may contain a second heteroatom selected from oxygen, sulfur or NR¹⁴ wherein R¹⁴ is hydrogen or (C₁-C₆)alkyl;

R¹² for each occurrence is independently hydrogen or (C₁-C₆)alkyl;

R¹³ is hydrogen, halo or (C₁-C₆)alkyl;

or R³ and R⁴ may be taken together with the carbons to which they are attached to form a compound of the formula

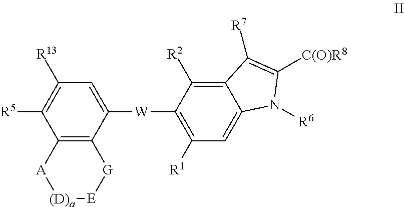

wherein a is 0, 1, 2 or 3;

A, D, E and G are each independently selected from the group consisting of CR¹⁶R¹⁷, NR¹⁸, oxygen or sulfur;

R¹⁶ and R¹⁷ for each occurrence are each independently selected from hydrogen or (C₁-C₆)alkyl; and

R¹⁸ is hydrogen, (C₁-C₆)alkyl, —C(O)R¹⁰ or —S(O)₂R¹⁰ wherein R¹⁰ is defined as above.

More particularly, the following compounds are useful in the methods of the present invention:
5-(4-Hydroxy-3-isopropyl-phenoxy)-4,6-dimethyl-1H-indole-2-carboxylic acid;
4,6-Dichloro-5-(4-hydroxy-3-isopropyl-phenoxy)-1H-indole-2-carboxylic acid;
5-(3-sec-butyl-4-hydroxy-phenoxy)-4,6-dimethyl-1H-indole-2-carboxylic acid;
5-[3-(4-Fluoro-benzyl)-4-hydroxy-phenoxyl]-4,6-dimethyl-1H-indole-2-carboxylic acid;

5-{3-[(4-Fluoro-phenyl)-hydroxy-methyl]-4-hydroxy-phenoxy}-4,6-dimethyl-1H-ondole-2-carboxylic acid;
5-[3-(2-Cyclopentyl-1-hydroxy-ethyl)-4-hydroxy-phenoxyl]-4,6-dimethyl-1H-indole-2-carboxylic acid;
5-[3-(4-Fluoro-benzoyl)-4-hydroxy-phenoxyl]-4,6-dimethyl-1H-indole-2-carboxylic acid;
5-[3-(Cyclobutyl-methyl-carbamoyl)-4-hydroxy-phenoxyl]-4,6-dimethyl-1H-indole-2-carboxylic acid;
5-(3-Cyclobutylmethanesulfonyl-4-hydroxy-phenoxy)-4,6-dimethyl-1H-indole-2-carboxylic acid;
4,6-Dichloro-5-(3-cyclobutylmethanesulfonyl-4-hydroxy-phenoxy)-1H-indole-2-carboxylic acid;
4,6-Dichloro-5-(4-hydroxy-3-isopropyl-phenoxy)-1-methyl-1H-indole-2-carboxylic acid;
4,6-Dichloro-5-(4-hydroxy-3-isopropyl-phenoxy)-3-methyl-1H-indole-2-carboxylic acid;
5-[3-(4-Fluoro-benzenesulfonyl)-4-hydroxy-phenoxy]-4,6-dimethyl-1H-indole-2-carboxylic acid;
5-[3-(4-Fluoro-benzenesulfonyl)-4-hydroxy-phenoxy]-3,4,6-trimethyl-1H-indole-2-carboxylic acid;
4,6-Dichloro-5-(3-cyclobutylsulfamoyl-4-hydroxy-phenoxy)-1H-indole-2-carboxylic acid;
4-Chloro-50(3-cyclopropylsulfamoyl-4-hydroxy-phenoxy)-5-methyl-1H-indole-2-carboxylic acid;
4,6-Dichloro-5-[4-hydroxy-3-(1-isopropyl-2-methyl-propylcarbamoyl)-phenoxy]-1H-indole-2-carboxylic acid;
5-[3-(4-Fluoro-benzenesulfonyl)-4-hydroxy-phenoxy]-1,4,6-trimethyl-1H-indole-2-carboxylic acid;

5-{3-[(4-Fluoro-phenyl)-hydroxy-methyl]-4-hydroxy-phenoxy)-3,4,6-trimethyl-1H-indole-2-carboxylic acid;
5-[3-(4-Fluoro-benzyl)-4-hydroxy-phenoxy]-3,4,6-trimethyl-1H-indole-2-carboxylic acid;
5-(3-Cyclopentylmethanesulfonyl-4-hydroxy-phenoxy)-4,6-dimethyl-1H-indole-2-carboxylic acid;
4,6-Dichloro-5-(3-cyclopropylsulfamoyl-4-hydroxy-phenoxy)-1H-indole-2-carboxylic acid;
5-(3-Cyclohexylmethanesulfonyl-4-hydroxy-phenoxy)-4,6-dimethyl-1H-indole-2-carboxylic acid;
5-(3-Cyclopropylsulfamoyl-4-hydroxy-phenoxy)-4,6-dimethyl-1H-indole-2-carboxylic acid;
5-(4-Hydroxy-3-isopropyl-phenoxy)-3,4,6-trimethyl-1H-indole-2-carboxylic acid;
5-(4-Hydroxy-3-isopropyl-phenoxy)-1,4,6-trimethyl-1H-indole-2-carboxylic acid;
4,6-Dichloro-5-[3-(4-fluoro-benzenesulfonyl)-4-hydroxy-phenoxy]-1H-indole-2-carboxylic acid;
4,6-Dichloro-5-[3-(4-fluoro-benzenesulfonyl-4-hydroxy-phenoxy]-3-methyl-1H-indole-2-carboxylic acid;
5-(3-Cyclobutylsulfamoyl-4-hydroxy-phenoxy)-4,6-dimethyl-1H-indole-2-carboxylic acid;
5-[4-Hydroxy-3-(1-isopropyl-2-methyl-propylcarbamoyl)-phenoxy]-4,6-dimethyl-1H-indole-2-carboxylic acid;
4,6-Dichloro-5-(3-[(4-fluoro-phenyl)-hydroxy-methyl]-4-hydroxy-phenoxy)-1H-indole-2-carboxylic acid;
5-(4-Hydroxy-2,3-dimethyl-phenoxy)-4,6-dimethyl-1H-indole-2-carboxylic acid;
4,6-Dichloro-5-(4-hydroxy-2,3-dimethyl-phenoxy)-1H-indole-2-carboxylic acid;
5-(7-Hydroxy-indan-4-yloxy)-4,6-dimethyl-1H-indole-2-carboxylic acid;
4,6-Dichloro-5-(7-hydroxy-indan-4-yloxy)-1H-indole-2-carboxylic acid;
4,6-Dichloro-5-(4-hydroxy-5,6,7,8-tetrahydro-naphthalen-1-yloxy)-1H-indole-2-carboxylic acid; and
5-(4-Hydroxy-5,6,7,8-tetrahydro-naphthalen-1-yloxy)-4,6-dimethyl-1H-indole-2-carboxylic acid.

Methods for making the thyromimetic compounds described above are disclosed in the above cited patent applications. All of the hereinabove and below cited references, U.S. patent applications, published European patent applications and published PCT International patent applications are hereby incorporated by reference herein in their entireties.

The compounds described herein can be used for the treatment of a nail, where the nail has been damaged or disfigured, including infections of the nail and various forms of damage (e.g., physical trauma or chemical injury), to increase the rate of re-growth of the damaged or disfigured nail. The compounds described herein can also be used for the treatment of healthy nails in normal individuals who desire to accelerate the growth of their nails.

The ability of a thyromimetic compound to bind thyroid hormone receptors may be demonstrated in standard assays known in the art, such as the Thyroid

Hormone Receptor Binding Assay, described at page 53 of published European application EP 1 088 819.

Also, as noted above, preferably, the compounds useful in the present invention are cardiac-sparing. Compounds may be tested for their cardiac-sparing properties using the following assay:

### Cardiotoxicity Assay

As is well known by those skilled in the art, thyroid hormones affect cardiac functioning, for example, by causing an increase in the heart rate as well as an increase in tissue mass, or hypertrophy. The ability of the compounds useful in the methods of the present invention to cause the thyroid hormone-like, cardiotoxic effects may be demonstrated according to the following protocol:

A. Experimental Summary

This in vivo screen is designed to evaluate the cardiac effects of compounds that are thyromimetic. The cardiac endpoints that are measured are heart weight and heart mitochondrial alpha-glycerophosphate dehydrogenase (“mGPDH”) activity. The protocol involves: (a) dosing rodents for about 6 days, (b) harvesting tissue and weighing it, (c) preparing a subcellular fraction of the tissue, enriched in mitochondria, and (d) subsequent assaying of enzyme activity thereby.

B. Preparation of Animals

A compound useful in the methods of the present invention, and a vehicle, or T₃ sodium salt, is administered topically or orally as a single daily dose given between about 3 p.m. to about 6 p.m. for about 6 days.

Animals are sacrificed by decapitation, tissues are dissected and weighed (for example, heart), then placed into 10 ml of cold homogenization buffer (0.25 M sucrose, 25 mM HEPES, pH 7.4, 0.5 mM EDTA, 0.5 mM AEBSF, 1 μg/ml leupeptin), stored on ice. Tissue homogenates are prepared using a Polytrone homogenizer (Kinematica AG, Switzerland), and then centrifuged at 15,000×g (11000 rpm, 10 minutes, at 4° C. using a Sorvall® SM-24 rotor (Dupont)), after which the supernatant is discarded. The pellet is resuspended in homogenization buffer containing 0.1% Triton-x 100 (6 mL), followed by sonication for 30 seconds (Branson Sonifier (Branson, Eagle Rd., Danbury, Conn. 06810), setting #2). Aliquots (1 ml in duplicate) are stored at −80° C., and 20 μl is placed in a separate tube for determination of the total protein concentration in the homogenate, using the BCA Protein Assay Kit (Pierce, 3747 No. Meridian Rd., Rockford, Ill. 61105).

The mGPDH enzyme assay is carried out by incubating a sample of the tissue homogenate (containing a range of protein amounts, from 10-100 ug) prepared as described above, in a buffer of the following composition: 225 mM mannitol, 75 mM sucrose, 20 mM HEPES, pH 7.4, 50 mM KH2PO₄, 1 mM KCN, 0.0025 mM rotenone, 0.025 mM menadione, 0.06 mM 2,6 dichlororindophenol. The assay is carried out at 37° C. in a Molecular Devices SpectraMax 96-well plate spectrophotometer (1311 Orleans Drive, Sunnyvale, Calif. 94089), by addition of substrate (α-glycerophosphate) to a final concentration of 50 mM. The decline in absorbance at 600 nm wavelength is measured frequently over the next 30-60 minutes. The enzyme activity is calculated from the change in absorbance at 600 nM versus time. Finally, the change in absorbance at 600 nm per unit of time is divided by the amount of protein used in the assay. By comparing the mGPDH enzyme activity in cardiac tissue from control animals dosed with a vehicle solution to the mGPDH enzyme activity in cardiac tissue from animals treated with a thyromimetic compound, the cardiac effect of the thyromimetic compound can be assessed. Another effect of a thyromimetic compound on the heart is hypertrophy. Cardiac hypertrophy in response to a thyromimetic compound can be assessed by comparing the heart weight in control animals dosed with a vehicle solution to the heart weight in animals dosed with a thyromimetic compound

There are known in the art other in vitro and in vivo assays models for understanding the biology of nail growth and for identifying the mechanisms that control nail growth and the nail growth cycle. These models are amenable to determining the effect of a compound useful in the methods of the present invention on nail growth.

The compounds of the present invention can be administered and nail growth measured by a variety of methods as described by R. Dawber and R. Baran, “Nail Growth,” CUTIS 39, 99-103 (1987); and D. L. Moffitt and D. A. R. de Berker, “Yellow nail syndrome: the nail that grows half as fast grows twice as thick,” Clinical and Experimental Dermatology, 25, 21-23 (2000). Additional methods are referenced in U. Runne and C. E. Orfanos, “The Human Nail,” Curr. Probl. Derm., vol. 9, pp. 102-149 (Karger, Basel 1981); and N. Orentreich et al., “The Effect of Aging on the Rate of Linear Nail Growth,” J. Invest. Dermatol., 73: 126-130 (1979).

In order to ensure that the compound of the present invention reaches the target tissues of the nail, it is necessary to prepare a formulation that can be applied directly to the surface of the nail, and in some cases, to the skin adjacent to and overlying the matrix at the base of the nail. The formulation will be designed such that it delivers drug through the nail plate or skin to the matrix. This can be achieved by including penetration enhancers in the formulation, such as oxacyclohexadecano-2-one.

The nail plate is thick, hard, dense and represents a formidable barrier for drugs to be able to penetrate in a therapeutically required quantity. Nail material is similar to the stratum corneum of the skin, being derived from keratin, which is highly disulfide-linked, and is approximately 100-fold thicker than the stratum corneum. In order to deliver a sufficient amount of drug into the nail matrix and nail bed, the permeability of the nail plate to the drug must be enhanced. Typically, an agent, such as cysteine, N-acetyl cysteine, and urea, is added to the formulation to increase drug permeability in the nail by breaking disulfide bonds in the nail keratin to increase drug penetration into and through the nail. Hence, a vehicle that contains an agent that enhances the permeability of the nail plate is highly desired to achieve adequate drug penetration to the nail matrix and nail bed. See, for example, the following references for additional information on various methods of administering drugs to nails as well as various formulations for administering drugs to nails, all of which are hereby incorporated by reference herein: U.S. Pat. No. 6,042,845; U.S. Pat. No. 6,231,875; published International patent application WO 01/15670; and published International patent application WO 99/49835.

The physical form of the formulation can be, for example, a solution, lacquer, nail polish, cream, ointment, foam, spray, artificial nail or medicated bandage. The formulation can be composed in such a way as to slowly release the compound over time. Also, the formulation may be suitable for injection into the nail by procedures known in the art. The formulation, dose and rate of release from the formulation will be such as to avoid sufficient compound from reaching the systemic circulation so as to produce unwanted side effects. To support this objective, the compounds that are preferred for use in the methods of the present invention will be those that undergo rapid metabolic conversion to inactive metabolites, and/or are rapidly cleared from the circulation, upon reaching the systemic circulation.

The methods of the present invention are performed by administering to a mammal (preferably a human) a compound as described herein and preferably, a pharmaceutically acceptable or cosmetically acceptable carrier. Preferably, in the methods of the present invention, the compounds are formulated into pharmaceutical or cosmetic compositions for use in treatment or prophylaxis of conditions, as described herein. Standard pharmaceutical formulation techniques are used, such as those disclosed in Remington's Pharmaceutical Sciences, Mack Publishing Company, Easton, Pa. (1990).

Typically, from about 0.0001% to about 10% (w/v), more preferably from about 0.0001% to about 1% (w/v), more preferably from about 0.0001% to about 0.1% (w/v), of a compound as described herein is administered per day for topical administration. However, it is understood that daily administration of a compound as described herein can be adjusted depending on various factors. The specific dosage of the compound to be administered, as well as the duration of treatment, and the specific method of administration are interdependent. The dosage and treatment regimen will also depend upon such factors as the specific compound used, the treatment indication, the efficacy of the compound, the personal attributes of the subject (such as, for example, weight, age, sex and medical condition of the subject), compliance with the treatment regimen, and the presence and severity of any side effects of the treatment.

According to the present invention, the compounds as described herein are co-administered with a pharmaceutically acceptable or cosmetically acceptable carrier (herein collectively described as a “carrier”). The term “carrier,” as used herein, means one or more compatible solid or liquid filler diluents, vehicles or encapsulating substances, which are suitable for administration to a mammal. The term “compatible,” as used herein, means that the components of the composition are capable of being commingled with a compound as described herein, and with each other, in a manner such that there is no interaction which would substantially reduce the efficacy of the composition under ordinary use situations. Carriers must, of course, be of sufficiently high purity and sufficiently low toxicity to render them suitable for administration to the mammal (preferably the human being) being treated. The carrier can itself be inert or it can possess pharmaceutical and/or cosmetic benefits of its own.

The compounds useful in the methods of the present invention may be formulated in any of a variety of forms suitable for topical administration. Preferably, the compounds useful in the methods of the present invention are administered topically. The carrier of the topical composition preferably aids penetration of the compounds as described herein into the nail to reach the environment of the nail matrix. Such topical compositions may be in any form including, for example, solutions, oils, creams, ointments, gels, lotions, pastes, milks, cleansers, moisturizers, sprays, aerosols, patches, lacquers, artificial nails and the like.

In topical compositions of the present invention, the compound as described herein may be in a form (e.g., a prodrug), which would more readily penetrate into the skin and then be converted to the active form upon reaching the desired skin layer. Also, the topical compositions containing a compound as described herein can be admixed with a variety of carrier materials well known in the art, such as, for example, water, alcohols, aloe vera gel, allantoin, glycerine, vitamin A and E oils, mineral oil, propylene glycol, PPG-2 myristyl propionate and the like. Other materials suitable for use in topical carriers include, for example, emollients, solvents, humectants, thickeners and powders. Examples of each of these types of materials, which can be used singly or as mixtures of one or more materials, are as follows:

Emollients include, for example, stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate and myristyl myristate. Propellants include, for example, propane, butane, isobutane, dimethyl ether, carbon dioxide and nitrous oxide. Solvents include, for example, ethyl alcohol, methylene chloride, isopropanol, castor oil, ethylene glycol monoethyl ether, diethylene glycol monobutyl ether, diethylene glycol monoethyl ether, dimethyl sulphoxide, dimethyl formamide and tetrahydrofuran. Humectants include, for example, glycerin, sorbitol, sodium 2-pyrrolidone-5-carboxylate, soluble collagen, dibutyl phthalate and gelatin. Powders include, for example, chalk, talc, fullers earth, kaolin, starch, gums, colloidal silicon dioxide, sodium polyacrylate, tetraalkyl ammonium smectites, trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, and ethylene glycol monostearate. These and other suitable agents for inclusion in the compositions of the present invention are synthesized by methods known in the art; and many are also commercially available.

The compounds used in the methods of the present invention may also be administered topically in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. A preferred formulation for topical delivery of the compounds used in the methods of the present invention utilizes liposomes such as described in Dowton et al., “Influence of Liposomal Composition on Topical Delivery of Encapsulated Cyclosporin A: I. An in vitro Study Using Hairless Mouse Skin”, S.T.P. Pharma Sciences, Vol. 3, pp. 404-407 (1993); Wallach and Philippot, “New Type of Lipid Vesicle: Novasome®”, Liposome Technology, Vol. 1, pp. 141-156 (1993); U.S. Pat. No. 4,911,928; and U.S. Pat. No. 5,834,014.

The above described compositions containing a compound as described herein may also optionally comprise an additional agent to accelerate or promote the rate of nail growth (nail growth promoter). This additional agent can be chosen from a wide variety of molecules which may function in different ways to enhance the nail growth effects of a compound used in the methods of the present invention. The following drugs (as summarized in U. Runne and C. E. Orfanos, Curr. Probl. Derm., vol. 9, pp. 102-149 (Karger, Basel 1981)) have been found to accelerate the rate of nail growth: gelatin, biotin, cystine and methionine, I-dopa. Other substances, such as certain nail polishes and vitamins, which are commercially available in drug stores and beauty salons, are also marketed as making nails stronger and less brittle, resulting in longer and stronger nails.

The formulation can include both a compound of the present invention and an antibacterial agent. The following are examples of antibacterial agents which can be used in combination with the compounds of the present invention: β-lactams, including carbapenems, cephamycins, penicillins and cephalosporins; Glycopeptides, including vancomycin and teicoplanin; Aminoglycosides, including streptomycins, neomycins, kanamycins and spectinomycin; Macrolides, including erythromycins, azithromycin and clarithromycin; Bacitracin; Clycoserine; Fosfomycin; Chloramphenicol; Tetracyclines; Fusidic acid; Polymyxins; Diaminopyrimidines; Lincosamides; Streptogramins; Mupirocin; Sulphonamides; Quinolones and fluoroquinolones; Novobiocin; Nitroimidazoles; Nitrofurans; Rifamycins, including rifampicin; and Oxazolidinones, including linezolid. This combination of a compound of the present invention and an antibacterial agent will eliminate an infection and accelerate the regrowth of a normal nail after it has been damaged by the infection.

The formulation can include both a compound of the present invention and an antifungal agent. The following are examples of antifungal agents which can be used in combination with the compounds of the present invention: Azoles, including fluconazole (DIFLUCAN), ketoconazole, itraconazole (SPORANOX), clotrimazole, miconazole, econazole, isoconazole, sulconazole, tioconazole and voriconazole; Allylamines, including terbinafine (LAMISIL); Amorolfine; Ciclopirox; Griseofulvin; Tolnaftate; Haloprogin; Candins, including cancidas and anidulafungin; and Polyenes, including amphotericin B (note, amphotericin would not be used for superficial infections). Another antifungal agent is ciclopirox, which is available commercially as a topical solution, known as Penlac Nail Lacquer. Preferred antifungal agents include the following: fluconazole, itraconazole, terbinafine and ciclopirox. This combination of a compound of the present invention and an antifungal agent will accelerate elimination of the fungal infection because the more rapidly growing nail will clear fungal spores more rapidly.

Additional methods of administering the compounds of the present invention, alone or in combination with an antifungal agent, and additional ways of formulating such compounds, either alone or in combination with an antifungal agent, are discussed in the following references, which are incorporated by reference herein: U.S. Pat. No. 6,281,239; U.S. Patent Application Publication US 2001/0046478; U.S. Pat. No. 5,696,164; and International Publication No. WO 99/53913.

In all of the foregoing, the compounds used in the methods of the present invention can be administered alone or as mixtures; and the compositions may further include additional drugs or excipients as appropriate for the indication, such as described above.

The present invention further relates to kits comprising a compound and/or composition as described herein and information and/or instructions by words, pictures, and/or the like, that use of the kit will provide treatment for nail growth in mammals (particularly humans). In addition or in the alternative, the kit may comprise a compound and/or composition as described herein and information and/or instructions regarding methods of application of the compound and/or composition, preferably with the benefit of increasing nail growth in mammals.

### EXAMPLES

In the examples below, “active ingredient” means a compound useful in the methods of the present invention, as described above.

### Example A

A composition for topical administration is made, comprising:

| COMPONENT | AMOUNT |
|---|---|
| Active Ingredient | 1% |
| Ethanol | 61% |
| Propylene Glycol | 19% |
| Dimethyl Isosorbide | 19% |

1. A method for increasing the rate of nail growth in a mammal which comprises the local administration of a thyromimetic compound to the mammal.

2. A method of claim 1 wherein the thyromimetic compound is administered to the nail and/or immediately surround tissue.

3. A method of claim 1 wherein the thyromimetic compound is administered topically.

4. A method for increasing the rate of nail growth in a mammal which comprises the administration to the mammal of an effective amount of a compound of the formula 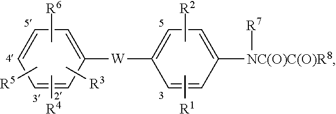
a prodrug thereof, a geometric or optical isomer thereof, or a pharmaceutically acceptable salt of said compound, said prodrug, or said isomer, wherein:
R¹, R² and R³ are each independently hydrogen, halogen, C₁₋₆ alkyl, trifluoromethyl, —CN, —OCF₃ or —OC₁₋₆ alkyl;
R⁴ is hydrogen, C₁₋₁₂ alkyl optionally substituted with one to three substitutents independently selected from Group Z, C₂₋₁₂ alkenyl, halogen, —CN, aryl, heteroaryl, C₃₋₁₀ cycloalkyl, heterocycloalkyl, —S(O)₂NR⁹R¹⁰, —C(O)NR⁹R¹⁰, —(C₁₋₆ alkyl) —NR⁹R¹⁰, —NR⁹C(O)R¹⁰, —NR⁹C(O)NR⁹R¹⁰, —NR⁹S(O)₂R¹⁰, —(C₁₋₆ alkyl) —OR¹¹, —OR¹¹ or —S(O)ₐR¹², provided that, where R⁵ is not fluoro, R⁴ is —S(O)₂NR⁹R¹⁰, —C(O)NR⁹R¹⁰, —(C₁₋₆ alkyl) —NR⁹R¹⁰, —NR⁹C(O)R¹⁰, —NR⁹C(O)NR⁹R¹⁰, —NR⁹S(O)₂R¹⁰, (C₁₋₆alkyl) —OR¹¹, —OR¹¹ or —S(O)ₐR¹²;
or R³ and R⁴ may be taken together to form a carbocyclic ring A of the formula —(CH₂)_{b}— or a heterocyclic ring A selected from the group consisting of -Q-(CH₂)_{c}— and —(CH₂)ⱼ-Q-(CH₂)ₖ— wherein Q is O, S or NR¹⁷, wherein said carbocyclic ring A and said heterocyclic ring A are each independently optionally substituted with one or more substituents independently selected from C₁₋₄ alkyl, halide or oxo;
R⁵ is fluoro, hydroxy, C₁₋₄ alkoxy or OC(O)R⁹;
or R⁴ and R⁵ may be taken together to form a heterocyclic ring B selected from the group consisting of —CR⁹═CR¹⁰—NH—, —N═CR⁹—NH—, —CR⁹═CH—O— and —CR⁹═CH—S—;
R⁶ is hydrogen, halogen, C₁₋₄ alkyl or trifluoromethyl;
R⁷ is hydrogen or C₁₋₆ alkyl;
R⁸ is —OR⁹ or —NR¹⁹R²⁰;
R⁹ and R¹⁰ for each occurrence are independently (A) hydrogen, (B) C₁₋₁₂ alkyl optionally substituted with one or more substituents independently selected from Group V, (C)C₂₋₁₂ alkenyl, (D) C₃₋₁₀ cycloalkyl optionally substituted with one or more substituents independently selected from C₁₋₆ alkyl, C₂₋₅ alkynyl, C₃₋₁₀ cycloalkyl, —CN, —NR¹³R¹⁴, oxo, —OR¹⁸, —COOR¹⁸ or aryl optionally substituted with X and Y, (E) aryl optionally substituted with X and Y, or (F) het optionally substituted with X and Y;
or R⁹ and R¹⁰ for any occurrence may be taken together to form a heterocyclic ring C optionally further containing a second heterogroup selected from the group consisting of —O—, —NR¹³— and —S—, and optionally further substituted with one or more substituents independently selected from C₁₋₅ alkyl, oxo, —NR¹³R¹⁴, —OR¹⁸, —C(O)₂R¹⁸, —CN, —C(O)R⁹, aryl optionally substituted with X and Y, het optionally substituted with X and Y, C₅₋₆ spirocycloalkyl, and a carbocyclic ring B selected from the group consisting of 5-, 6-, 7- and 8-membered partially and fully saturated, and unsaturated carbocyclic rings, and including any bicyclic group in which said carbocyclic ring B is fused to a carbocyclic ring C selected from the group consisting of 5-, 6-, 7- and 8-membered partially and fully saturated, and unsaturated carbocyclic rings;
R¹¹ is C₁₋₁₂ alkyl optionally substituted with one or more substituents independently selected from Group V, C₂₋₁₂ alkenyl, C₃₋₁₀ cycloalkyl, trifluoromethyl, difluoromethyl, monofluoromethyl, aryl optionally substituted with X and Y, het optionally substituted with X and Y, —C(O)NR⁹R¹⁰ or —C(O)R⁹;
R¹² is C₁₋₁₂ alkyl optionally substituted with one or more substituents independently selected from Group V, C₂₋₁₂ alkenyl, C₃₋₁₀ cycloalkyl, aryl optionally substituted with X and Y, or het optionally substituted with X and Y;
R¹³ and R¹⁴ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁵R¹⁸ or C₃₋₁₀ cycloalkyl;
R¹⁵ and R¹⁶ for each occurrence are independently hydrogen, C₁₋₆ alkyl, C₃₋₁₀ cycloalkyl or aryl optionally substituted with X and Y;
R¹⁷ is hydrogen, C₁₋₆ alkyl, —COR⁹ or —SO₂R⁹;
R¹⁸ is hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁸R¹⁸, —(C₁₋₄ alkyl) —(C₁₋₄ alkoxy) or C₃₋₁₀ cycloalkyl;
R¹⁹ is hydrogen or C₁₋₆ alkyl;
R²⁰ is hydrogen or C₁₋₆ alkyl;
W is O, S(O)_{d}, CH₂ or NR⁹;
Group Z is C₂₋₆ alkenyl, C₂₋₆ alkynyl, halogen, —CF₃, —OCF₃, hydroxy, oxo, —CN, aryl, heteroaryl, C₃₋₁₀ cycloalkyl, heterocycloalkyl, —S(O)ₐR¹², —S(O)₂NR⁹R¹⁰, —C(O)R⁹R¹⁰, and —NR⁹R¹⁰;
Group V is halogen, —NR¹³R¹⁴, —OCF₃, —OR⁹, oxo, trifluoromethyl, —CN, C₃₋₁₀ cycloalkyl, aryl optionally substituted with X and Y, and het optionally substituted with X and Y;
het for each occurrence is a heterocyclic ring D selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring D is fused to a benzene ring or a heterocyclic ring E selected from the group consisting of 4-, 5-, 6-, 7- and 8-membered partially and fully saturated, and unsaturated, heterocyclic rings containing from one to four heteroatoms independently selected from the group consisting of N, O and S;
X and Y for each occurrence are independently (A) hydrogen, (B) halogen, (C) trifluoromethyl, (D) —OCF₃, (E) —CN, (F) C₁₋₆ alkyl optionally substituted with one or more substituents independently selected from the group consisting of halogen, —OCF₃, —CF₃ and phenyl, (G) C₁₋₆ alkoxy, (H) aryl optionally substituted with one or more substituents independently selected from the group consisting of halogen, —OCF₃, —CF₃, C₁₋₄ alkyl and C₁₋₄ alkoxy, (l) —C(O)₂R¹³, (J) —C(O)NR¹³R¹⁴, (K) —C(O)R¹³, (L) —NR¹³C(O)NR¹³R¹⁴ and (M) —NR¹³C(O)R¹⁴;
or X and Y for any occurrence in the same variable may be taken together to form (a) a carbocyclic ring D of the formula —(CH₂)ₑ— or (b) a heterocyclic ring F selected from the group consisting of —O(CH₂)_{f}O—, (CH₂)_{g}NH— and —CH═CHNH—;
a and d are each independently 0, 1 or 2;
b is 3, 4, 5, 6 or 7;
c, f, g, j and k are each independently 2, 3, 4, 5 or 6; and
e is 3, 4, 5, 6 or 7.

5. A method of claim 4 wherein the compound is selected from the group consisting of:
N-[3-chloro-4-(3-cyclopropylsulfamoyl-4-hydroxy-phenoxy)-5-methyl-phenyl]-oxamic acid;
N-[4-(3-cyclopropylsulfamoyl-4-hydroxy-phenoxy)-3,5-dimethyl-phenyl]-oxamic acid;
N-{4-[3-(cyclobutyl-methyl-carbamoyl)-4-hydroxy-phenoxy]-3,5-dimethyl-phenyl}-oxamic acid;
N-{3-chloro-4-[3-(cyclobutyl-methyl-carbamoyl)-4-hydroxy-phenoxy]-5-methyl-phenyl}-oxamic acid;
N-[4-(7-hydroxy-indan-4-yloxy)-3,5-dimethyl-phenyl]-oxamic acid;
N-[3,5-dichloro-4-(3-(cyclobutyl-methyl-carbamoyl)-4-hydroxy-phenoxy]-phenyl)-oxamic acid;
N-[3,5-dichloro-4-(3-cyclopentanesulfonyl-4-hydroxy-phenoxy)-phenyl]-oxamic acid;
N-[3,5-dichloro-4-(3-cyclopropylmethanesulfonyl-4-hydroxy-phenoxy)-phenyl]-oxamic acid;
N-[3,5-dichloro-4-(3-cyclobutylmethanesulfonyl-4-hydroxy-phenoxy)-phenyl]-oxamic acid;
N-[4-(3-cyclopropylmethanesulfonyl-4-hydroxy-phenoxy)-3,5-dimethyl-phenyl]-oxamic acid;
N-[3-chloro-4-(3-cyclobutylmethanesulfonyl-4-hydroxy-phenoxy)-5-methyl-phenyl]-oxamic acid;
N-[4-(3-cyclobutylmethanesulfonyl-4-hydroxy-phenoxy)-3,5-dimethyl-phenyl]-oxamic acid;
N-[4-(3-cyclopentylmethanesulfonyl-4-hydroxy-phenoxy)-3,5-dimethyl-phenyl]-oxamic acid;
N-[3-chloro-4-(3-cyclopentylmethanesulfonyl-4-hydroxy-phenoxy)-5-methyl-phenyl]-oxamic acid;
N-[3,5-dichloro-4-(3-cyclopentylmethanesulfonyl-4-hydroxy-phenoxy)-phenyl]-oxamic acid;
N-[4-(3-cyclohexylmethanesulfonyl-4-hydroxy-phenoxy)-3,5-dimethyl-phenyl]oxamic acid;
N-[3-chloro-4-(3-cyclohexylmethanesulfonyl-4-hydroxy-phenoxy)-5-methyl-phenyl]-oxamic acid;
N-[3,5-dichloro-4-(3-cyclohexylmethanesulfonyl-4-hydroxy-phenoxy)-phenyl]-oxamic acid;
N-[3,5-dichloro-4-[3-(4-fluoro-benzenesulfonyl)-4-hydroxy-phenoxy)-phenyl]-oxamic acid;
N-(4-[3-(4-fluoro-benzenesulfonyl)-4-hydroxy-phenoxy]-3,5-dimethyl-phenyl]-oxamic acid; and
N-{3-chloro-4-[3-(4-fluoro-benzenesulfonyl)-4-hydroxy-phenoxy]-5-methyl-phenyl}-oxamic acid.

6. A method for increasing the rate of nail growth in a mammal which comprises the administration to the mammal of an effective amount of a compound of the formula 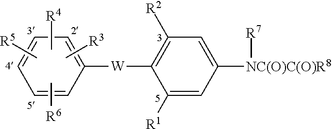
a prodrug thereof, a geometric or optical isomer thereof, or a pharmaceutically acceptable salt of said compound, said prodrug, or said isomer, wherein:
R¹ and R² are independently halogen, C₁₋₈ alkyl, —CN or C₁₋₈ perfluoroalkyl;
provided that at least one of R¹ and R² is —CN;
R³ is hydrogen or C₁₋₈ alkyl;
R⁴ is halogen, C₁₋₈ perfluoroalkyl, C₁₋₈ alkyl, C₁₋₈ alkanoyl, hydroxy-(C₁₋₈ alkyl), aryl optionally substituted with Y and Z, aryl-(C₁₋₈ alkyl), carbocyclic aroyl optionally substituted with Y and Z, C₃₋₁₀ cycloalkyl optionally substituted with Y and Z, or C₃₋₁₀ cycloalkyl-(C₁₋₈ alkyl);
or R⁴ is the radical 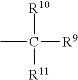
wherein: R⁹ is hydrogen, C₁₋₈ alkyl, aryl optionally substituted with Y and Z, aryl-(C₁₋₈ alkyl), C₃₋₁₀ cycloalkyl optionally substituted with Y and Z, or C₃₋₁₀ cycloalkyl-(C₁₋₈ alkyl); R¹⁰ is —OR¹⁴; R¹¹ is hydrogen or C₁₋₈ alkyl; or R¹⁰ and R¹¹ may be taken together with the carbon atom to which they are attached to form a carbonyl group;
R⁵ is hydroxy, esterified hydroxy or etherified hydroxy;
R⁶ is hydrogen, halogen, C₁₋₈ alkyl or C₁₋₈ perfluoroalkyl;
R⁷ is hydrogen, C₁₋₈ alkyl or C₁₋₈ perfluoroalkyl;
R⁸ is —OR¹² or —NR¹²R¹³;
R¹² and R¹³ are each independently hydrogen or C₁₋₈ alkyl;
R¹⁴ is hydrogen, C₁₋₈ alkyl or C₁₋₈ acyl;
X is O, S(O)ₐ, C═O or NR¹⁵;
a is 0, 1 or 2;
R¹⁵ is hydrogen or C₁₋₈ alkyl;
Y and Z for each occurrence are independently (a) hydrogen, (b) halogen, (c) trifluoromethyl, (d) —OCF₃, (e) —CN, (f) C₁₋₆ alkyl optionally substituted with one or more substituents independently selected from the group consisting of halogen, —OCF₃, —CF₃ and phenyl, (g) C₁₋₆ alkoxy, (h) aryl optionally substituted with one or more substituents independently selected from the group consisting of halogen, —OCF₃, —CF₃, C₁₋₄ alkyl and C₁₋₄ alkoxy, (i) —C(O)₂R¹⁶, (j) —C(O)NR¹⁶R¹⁷, (k) —C(O)R¹⁶, (l) —NR¹⁶C(O)NR¹⁶R¹⁷ or (m) —NR¹⁶C(O)R¹⁷; or Y and Z for any occurrence may be taken together to form (a) a carbocycle of the formula —(CH₂)_{b}, or (b) a heterocycle selected from the group consisting of —O(CH₂)_{c}O—, —(CH₂)_{d}NH— and —CH═CHNH—;
b is 3, 4, 5, 6 or 7;
c and d are each independently 2, 3, 4, 5 or 6;
R¹⁶ and R¹⁷ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁸R¹⁸ or C₃₋₁₀ cycloalkyl;
het for each occurrence is a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring is fused to a benzene ring or a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S; and
R¹⁸ and R¹⁹ for each occurrence are independently hydrogen, C₁₋₆ alkyl, C₃₋₁₀ cycloalkyl or aryl optionally substituted with Y and Z.

7. A method for increasing the rate of nail growth in a mammal which comprises the administration to the mammal of an effective amount of a compound of the formula 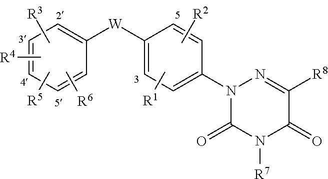
an isomer thereof, a prodrug of said compound or isomer, or a pharmaceutically acceptable salt of said compound, isomer or prodrug;
wherein W is (a) —O—, (b) —S(O)ₘ—, (c) —NR³⁰—, (d) —C(O) —, (e) —HC═CH—, (f) —CH₂—, (g) —CHF—, (h) —CF₂— or (i) —CH(OH) —;
R¹ and R² are independently (a) hydrogen, (b) halogen, (c) —(C₁-C₆)alkyl, (d) —CN, (e) —OR¹² or (f) -trifluoromethyl;
R³ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁰, (k) —C(O)R¹⁶, (l) —NR²¹C(O) —NR²¹R²², (m) —NR¹⁹—C(O)R²⁰ or (n) —NR¹⁷R¹⁸;
R⁴ is (a) —C(R¹⁴)(R¹⁵)(R¹⁶), (b) —(C₀-C₃)alkyl-NR¹⁷R¹⁸, (c) —C(O)NR¹⁹R²⁹, (d) —NR¹⁹—C(O) —R²⁰, (e) —(C₀-C₃)alkyl-NR²¹—C(O) —NR²¹R²², (f) —S(O)ₘ—R²², (g) —S(O)₂—NR²¹R²², (h) —NR²¹—S(O)₂—R²², (i) -aryl, (j) -het, (k) —OR³³ or (l) halogen; provided that in substituents (f) and (h), R²² is other than —OR³⁴; and provided that when substituent (b) is —(C₀)alkyl-NR¹⁷R¹⁸, R¹⁸ is other than —C(O) —R²⁸ or —S(O)₂—R²⁹;
or R³ and R⁴ may be taken together to form a carbocyclic ring of Formula —(CH₂)_{b}— or a heterocyclic ring selected from the group consisting of -Q-(CH₂)_{c}— and —(CH₂)ⱼ-Q-(CH₂)ₖ— wherein Q is O, S or NR²⁵; wherein said carbocyclic ring is optionally substituted with one or more substituents independently selected from Group V; and wherein said heterocyclic ring is optionally substituted with one or more substituents independently selected from Group Z;
R⁵ is —OR²³;
or R⁴ and R⁵ may be taken together to form a heterocyclic ring selected from the group consisting of —CR³¹═CR³²—NH—, —N═CR³¹—NH—, —CR³¹═CR³²—O— and —CR³¹═CR³²—S—;
R⁶ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁹, (k) —C(O)R¹⁶, (l) —NR²¹C(O)NR²¹R²², (m) —NR¹⁹—C(O)R²⁰ or (n) —NR¹⁷R¹⁸;
R⁷ is (a) hydrogen, (b) —(C₁-C₄)alkyl wherein each carbon atom is optionally substituted with 1 to 3 halo atoms or (c) —(CH₂)ₙCOOR⁹;
R⁸ is (a) hydrogen, (b) —(C₁-C₆)alkyl, (c) —C(O) —OR⁹, (d) —C(O)NR¹⁰R¹¹ or (e) —CN; provided that in substituent (c), R⁹ is other than methyl or ethyl; and provided that in substitutent (d), R¹⁰ and R¹¹ are not both hydrogen;
R⁹ is (a) —(C₁-C₁₂)alkyl optionally substituted with one or more substitutents independently selected from Group V, (b) —(C₂-C₁₂)alkenyl optionally substituted with phenyl, (c) —(C₂-C₁₂)dialkenyl, (d) —(C₃-C₁₀)cycloalkyl, (e) -aryl or (f) -het;
R¹⁰ and R¹¹ are independently (a) hydrogen, (b) —(C₁-C₁₂)alkyl optionally substituted with one or more substituents independently selected from Group V, (c) —(C₃-C₁₀)cycloalkyl optionally substituted with one or more substituents independently selected from Group V, (d) —(C₂-C₁₂)alkenyl or (e) -het;
or R¹⁰ and R¹¹ for any occurrence may be taken together with the nitrogen atom to which are they attached to form het;
R¹² is (a) hydrogen or (b) —(C₁-C₆)alkyl wherein each carbon atom is optionally substituted with 1 to 3 fluoro atoms;
R¹³ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) —NR¹⁷R¹⁸, (e) -aryl or (f) -het;
R¹⁴ is (a) hydrogen, (b) —(C₁-C₆)alkyl or (c) —O—R³⁴;
R¹⁵ is (a) hydrogen or (b) —(C₁-C₆)alkyl;
or R¹⁴ and R¹⁵ are taken together with the carbon atom to which they are attached to form a carbonyl group;
R¹⁶ is (a) hydrogen, (b) —(C₁-C₆)alkyl wherein each carbon atom is optionally substituted with 1 to 3 fluoro atoms, (c) —(C₀-C₆)alkyl-(C₃-C₁₀)cycloalkyl, (d) —(C₀-C₆)alkyl-aryl or (e) —(C₀-C₆)alkyl-het;
R¹⁷ is (a) hydrogen, (b) —(C₁-C₁₂)alkyl optionally substituted with one or more substituents independently selected from Group V, (c) -aryl, (d) -het, (e) —OR³⁴ or (f) —(C₃-C₁₀)cycloalkyl;
R¹⁸ is (a) hydrogen, (b) —(C₁-C₁₂)alkyl optionally substituted with one or more substituents independently selected from Group V, (c) -aryl, (d) -het, (e) —C(O) —R²⁸, (f) —S(O)₂—R²⁹, (g) —OR³⁴ or (h) —(C₃-C₁₀)cycloalkyl;
or R¹⁷ and R¹⁸ for any occurrence are taken together with the nitrogen atom to which they are attached to form het;
R¹⁹ and R²⁰ for each occurrence are independently (a) hydrogen, (b) —(C₁-C₁₂)alkyl optionally substituted with one or more substituents independently selected from Group V, (c) —(C₀-C₆)alkyl-aryl, (d) —(C₀-C₆)alkyl-het, (e) —C(O) —NR²⁶R²⁷, (f) —C(O) —R²⁸, (g) —S(O)₂—R²⁹, (h) —OR³⁴ or (i) —(C₃-C₁₀)cycloalkyl;
or R¹⁹ and R²⁰ for any occurrence are taken together with the nitrogen atom to which they are attached to form het;
R²¹ and R²² for each occurrence are independently (a) hydrogen, (b) —(C₁-C₁₂)alkyl optionally substituted with one to three substituents independently selected from Group V, (c) -aryl, (d) -het, (e) —(C₃-C₁₀)cycloalkyl or (f) —OR³⁴;
or R²¹ and R²² are taken together with the nitrogen atom to which they are attached to form het;
R²³ is (a) hydrogen, (b) —(C₁-C₄)alkyl optionally substituted with one or more substituents independently selected from Group V or (c) —C(O) —R²⁴;
R²⁴ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R²⁵ for each occurrence is independently (a) hydrogen, (b) —(C₁-C₆)alkyl, (c) —COR²³ or (d) —SO₂R²⁹;
R²⁶ and R²⁷ for each occurrence are independently (a) hydrogen, (b) —(C₁-C₆)alkyl, (c) —(C₃-C₁₀)cycloalkyl, (d) —(C₀-C₆)alkyl-aryl, or (e) —(C₀-C₆)alkyl-het,
R²⁸ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R²⁹ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) -aryl or (e) -het;
R³⁰ is (a) hydrogen, (b) —(C₁-C₁₂)alkyl optionally substituted with one or more substituents independently selected from Group V, (c) —(C₁-C₁₂)alkenyl, (d) —(C₃-C₁₀)cycloalkyl, (e) —C(O) —R³¹ or (f) —S(O)ₘ—R³²;
R³¹ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl, (f) -het or (g) —OR³⁴;
R³² is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R³³ is (a) —(C₀-C₆)alkyl-aryl, (b) —(C₀-C₆)alkyl-het, (c) —(C₇-C₁₂)alkyl optionally substituted with one or more substituents independently selected from Group V, (d) —(C₁-C₆)alkyl wherein at least one carbon atom is substituted with 1 to 3 fluoro atoms, (e) —(C₂-C₁₂)alkenyl or (f) —(C₃-C₁₀)cycloalkyl;
R³⁴ is (a) -aryl, (b) -het, (c) —(C₁-C₁₂)alkyl optionally substituted with one or more substituents independently selected from Group V, (d) —(C₂-C₁₂)alkenyl or (e) —(C₃-C₁₀)cycloalkyl;
—(C₃-C₁₀)cycloalkyl for each occurrence is a fully or partially saturated mono-, bi- or tricyclic ring containing three to ten carbon atoms; wherein in the bicyclic ring, a monocyclic cycloalkyl ring is spiro used to another cycloalkyl ring or is fused via two carbon atoms to a benzene ring or another cycloalkyl ring; and wherein in the tricyclic ring, a bicyclic ring is spiro used to a cycloalkyl ring or is fused via two atoms to a benzene ring or another cycloalkyl ring;
said —(C₃-C₁₀)cycloalkyl optionally contains one to three bridging atoms independently selected from carbon, oxygen, sulfur and nitrogen; said bridging atoms are attached to two carbon atoms in the ring; and said bridging atoms are optionally substituted with one to three groups independently selected from —(C₁-C₆)alkyl and hydroxy;
said cycloalkyl ring is optionally substituted on one ring if the moiety is monocyclic, on one or both rings if the moiety is bicyclic, or on one, two or three rings if the moiety is tricyclic, with one or more substitutents independently selected from Group V;
Group V is (a) —(C₁-C₆)alkyl optionally substituted with one or two hydroxy, (b) —(C₂-C₅)alkynyl, (c) -halogen, (d) —NR³⁵R³⁶, (e) —NO₂, (f) —OCF₃, (g) —OR³⁷, (h) —SR³⁷, (i) -oxo, (j) -trifluoromethyl, (k) —CN, (l) —C(O)NR³⁵—OH, (m) —COOR³⁵, (n) —O—C(O) —(C₁-C₆)alkyl, (O) —(C₃-C₁₀)cycloalkyl optionally substituted with CN, (p) —(C₀-C₆)alkyl-aryl, (q) —(C₀-C₆)alkyl-het, (r) —C(O) —(C₁-C₆)alkyl or (s) —C(O)-aryl;
R³⁵ and R³⁶ for each occurrence are independently (a) hydrogen, (b) —(C₁-C₆)alkyl or (c) —(C₀-C₆)alkyl-aryl;
R³⁷ is (a) hydrogen, (b) —(C₁-C₆)alkyl optionally substituted with one or more halo, hydroxy or methoxy, (c) —(C₀-C₆)alkyl-aryl or (d) —(C₀-C₆)alkyl-het;
aryl is (a) phenyl optionally substituted with one or more substituents independently selected from Group Z; (b) naphthyl optionally substituted with one or more substituents independently selected from Group Z or (c) biphenyl optionally substituted with one or more substituents independently selected from Group Z;
het for each occurrence is a 4-, 5-, 6-, 7- and 8-membered fully saturated, partially saturated or fully unsaturated mono-, bi- or tricyclic heterocyclic ring containing from one to four heteroatoms independently selected from the group consisting of oxygen, sulfur and nitrogen; wherein in the bicyclic ring, a monocyclic heterocyclic ring is spiro fused to a —(C₃-C₈)cycloalkyl ring or to another heterocyclic ring which is fully or partially saturated; or is fused via two atoms to a benzene ring, a —(C₃-C₈)cycloalkyl ring or another heterocyclic ring; and wherein in the tricyclic ring, a bicyclic ring is Spiro fused to a —(C₃-C₈)cycloalkyl ring or to another heterocyclic ring which is fully or partially saturated; or is fused via two atoms to a benzene ring, a (C₃-C₆)cycloalkyl ring, or another heterocyclic ring;
said het optionally contains one to three bridging atoms independently selected from oxygen, sulfur and nitrogen; said bridging atoms are attached to two other atoms in the ring; and said bridging atoms are optionally substituted with one to three groups independently selected from —(C₁-C₆)alkyl and hydroxy;
said het optionally has one or two oxo groups substituted on carbon or one or two oxo groups substituted on sulfur;
said het is optionally substituted on carbon or nitrogen, on one ring if the moiety is monocyclic, on one or both rings if the moiety is bicyclic, or on one, two or three rings if the moiety is tricyclic, with one or more substituents independently selected from Group Z;
Group Z for each occurrence is independently (a) hydrogen, (b) halogen, (c) trifluoromethyl, (d) hydroxy, (e) —OCF₃, (f) —CN, (g) —NO₂, (h) —(C₁-C₆)alkyl optionally substituted with one or more substituents independently selected from the group consisting of hydroxy, halogen, —OCF₃ and —CF₃, (i) —(C₂-C₆)alkenyl optionally substituted with phenyl, (j) —(C₂-C₅)alkynyl, (k) —(C₁-C₆)alkoxy, (l) —(C₀-C₈)alkyl-phenyl optionally substituted with one or more substituents independently selected from the group consisting of halogen, —OCF₃, —CF₃, —(C₁-C₄)alkyl, —(C₁-C₄)alkoxy and —C(O)CH₃, (m) —(C₀-C₆)alkyl-naphthyl optionally substituted with one or more substituents independently selected from the group consisting of halogen, —OCF₃, —CF₃, —(C₁-C₄)alkyl, —(C₁-C₄)alkoxy and —C(O)CH₃, (n) —C(O)₂R³⁵, (O) —(C₀-C₈)alkyl-C(O)NR³⁵R³⁶, (p) —(C₀-C₆)alkyl-C(O)R³⁸, (q) —NR³⁵R³⁶, (r) —NR³⁵—C(O)NR³⁵R³⁶, (s) —NR³⁵—C(O)R³⁶, (t) —OR³⁷, (u) —SR³⁷, (v) —(C₃-C₁₈)cycloalkyl, (w) —(C₀-C₆)alkyl-pyridinyl optionally substituted with one or more —(C₁-C₆)alkyl which is optionally substituted with one or more substituents independently selected from the group consisting of hydroxy and halo, (x) —(C₀-C₆)alkyl-piperidinyl optionally substituted with one or more —(C₁-C₆)alkyl which is optionally substituted with one or more substituents independently selected from hydroxy and halo, (y) —SO₂—R³⁷, (z) —SO₂—NR³⁵R³⁶ or (a1) —S-phenyl-CH₂OH;
R³⁸ is (a) —(C₁-C₆)alkyl, (b) —(C₀-C₆)alkyl-phenyl, (c) —(C₀-C₆)alkyl-phenanthrenyl optionally substituted with one to three CF₃, (d) —(C₀-C₆)alkyl-pyrrolidinyl or (e) —(C₀-C₆)alkyl-morpholinyl;
or any two Z Groups for any occurrence in the same variable may be taken together to form (a) a carbocyclic ring of the formula —(CH₂)ₑ— or (b) a heterocyclic ring selected from the group consisting of —O(CH₂)_{f}O—, —(CH₂)_{g}NH— and —CH═CHNH—;
m is 0, 1 or 2;
n is 0,1, 2 or 3;
b is 3, 4, 5, 6 or 7;
c, f, g, j and k are each independently 2, 3, 4, 5 or 6; and
e is 3, 4, 5, 6 or 7;
provided that in a compound of the above formula: 1) the substituent —C(R¹⁴)(R¹⁵)(R¹⁶) in R⁴ is other than (C₁-C₄)alkyl; and 2) R⁴ is halo only when R⁸ is —C(O) —OR⁹ or —C(O)NR¹⁰R¹¹.

8. A method of claim 7 wherein the compound is selected from the group consisting of:
8-[[5-[2,6-dichloro-4-(4,5-dihydro-3,5-dioxo-1,2,4-triazine-2(3H)-yl)phenoxy]-2-hydroxyphenyl]sulfonyl]-spiro[8-azabicyclo[3.2.1]octane-3,2′-(3′H)-dihydro-furan];
2-{3,5-dichloro-4-[3-(3,3-dimethyl-piperidine-1-sulfonyl)-4-hydroxy-phenoxy]-phenyl}-2H-[1,2,4]triazine-3,5-dione;
2-{3,5-dichloro-4-[4-hydroxy-3-(3-methyl-3-phenyl-piperidine-1-sulfonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzenesulfonamide;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,3-dimethyl-piperidine-1-carbonyl)-4-hydroxy-phenoxy]-phenyl}-2H-[1,2,4]triazine-3,5-dione;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[4-hydroxy-3-(3-methyl-3-phenyl-piperidine-1-carbonyl)-phenoxy]-phenyl}-2H-[1,2,4]triazine-3,5-dione;
5-[2,6-dichloro-4-(3,5-dioxo-4,5-dihydro-3H-[1,2,4]triazin-2-yl)-phenoxy]-N-(6,6-dimethyl-bicyclo[3.1.1]hept-2-yl)-2-hydroxy-benzamide;
2-{3,5-dichloro-4-[3-(3,5-dimethyl-piperidine-1-carbonyl)-4-hydroxy-phenoxy]-phenyl}-2H-[1,2,4]triazine-3,5-dione;
2-{3,5-dichloro-4-[4-hydroxy-3-(piperidine-1-carbonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,4-dihydro-1H-isoquinoline-2-carbonyl)-4-hydroxy-phenoxy]-phenyl}-2H-[1,2,4]triazine-3,5-dione;
2-{4-[3-(4-fluoro-benzyl)-4-hydroxy-phenoxy]-3,5-dimethyl-phenyl}-2H-[1,2,4]triazine-3,5-dione; and
2-{3,5-dichloro-4-[3-(4-fluoro-benzoyl)-4-hydroxy-phenoxy]-phenyl}-2H-[1,2,4]triazine-3,5-dione.

9. A method for increasing the rate of nail growth in a mammal which comprises the administration to the mammal of an effective amount of a compound of the formula 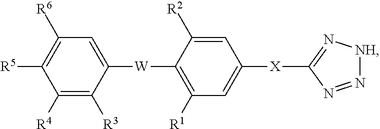
or a stereoisomer, a pharmaceutically acceptable salt or prodrug thereof, or a pharmaceutically acceptable salt of the prodrug, wherein:
W is O, S, SO, SO₂, CH₂, CF₂, CHF, C(═O), CH(OH), NR^{a}, or 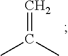
X is O, CH₂, CH₂CH₂, S, SO, SO₂, CH₂NR^{a}, NR^{a}, or a bond;
each R^{a} is independently hydrogen, C₁-C₆alkyl, or C₁-C₆alkyl substituted with one substituent selected from C₃-C₆cycloalkyl or methoxy;
R¹, R², R³ and R⁶ are independently hydrogen, halogen, C₁-C₈alkyl, —CF₃, —OCF₃, —OC₁-C₈alkyl, or —CN;
R⁴ is hydrogen, C₁-C₁₂alkyl, [C₁-C₁₂alkyl that is substituted with from one to three substituents independently selected from Group V], C₂-C₁₂ alkenyl, C₂-C₁₂ alkynyl, halogen, —CN, —OR^{b}, —SR^{c}, —S(═O)R^{c}, —S(═O)₂R^{c}, aryl, heteroaryl, C₃-C₁₀ cycloalkyl, heterocycloalkyl, —S(═O)₂NR^{c}R^{d}, —C(═O)NR^{c}R^{d}, —C(═O)OR^{c}, —NR^{a}C(═O)R^{d}, —NR^{a}C(═O)NR^{c}R^{d}, —NR^{a}S(═O)₂R^{d}, —NR^{a}R^{d}, —C(═O)R^{c},
or R³ and R⁴ may be taken together with the carbon atoms to which they are attached to form an unsubstituted or substituted carbocyclic ring of formula —(CH₂)ᵢ— or an unsubstituted or substituted heterocyclic ring selected from the group consisting of -Q-(CH₂)ⱼ— and —(CH₂)ₖ-Q-(CH₂)ₗ— wherein Q is O, S or NR^{a}; i is 3, 4, 5, 6 or 7; j is 2, 3, 4, 5, or 6; k and l are each independently 1, 2, 3, 4, or 5, and any substituents up to four are selected from C₁-C₄alkyl, —OR^{b}, oxo, —CN, phenyl, or —NR^{a}R^{g};
R^{b} is hydrogen, C₁-C₁₂alkyl, [C₁-C₁₂alkyl substituted with one to three substituents independently selected from Group V], aryl, heteroaryl, C₃-C₁₀ cycloalkyl, heterocycloalkyl, —C(═O)NR^{c}R^{d}, or —C(═O)R^{f};
R^{c} and R^{d} are each independently selected from hydrogen, C₁-C₁₂alkyl, [C₁-C₁₂alkyl substituted with one to three substituents independently selected from Group VI], C₂-C₁₂alkenyl, C₂-C₁₂alkynyl, aryl, heteroaryl, C₃-C₁₀ cycloalkyl, heterocycloalkyl,
or R^{c} and R^{d} may together along with the atom(s) to which they are attached form a 3-10 membered unsubstituted or substituted heterocyclic ring, which may contain a second heterogroup selected from O, NR^{e}, or S, wherein any substitutents up to four are selected from C₁-C₄alkyl, —OR^{b}, oxo, —CN, phenyl, or —NR^{a}R^{g};
R⁵ is —OH, —OC₁-C₆alkyl, —OC(═O)R^{f}, —F, —C(═O)OR^{c},
or R⁴ and R⁵ may together with the atom(s) to which they are attached form a heterocyclic ring selected from the group consisting of —CR^{c}═CR^{a}—NH—, —N═CR^{a}—NH—, —CR^{c}═CR^{a}—O—, —CR^{c}═CR^{a}—S—, —CR^{c}═N—NH—, or —CR^{a}═CR^{a}—CR^{a}═N—;
Group V is halogen, —CF₃, —OCF₃, hydroxy, oxo, C₁-C₆alkoxy, —CN, aryl, heteroaryl, C₃-C₁₀cycloalkyl, heterocycloalkyl, —S(═O)R^{f}, —S(═O)₂R^{f}, [—S(═O)₂NR^{a}R^{f}, wherein R^{a} and R^{f} may together along with the atom(s) to which they are attached form a 3-8 membered heterocyclic ring, which may contain a second heterogroup selected from O, NR^{a} or S], —NR^{a}R^{g}, or [—C(═O)NR^{a}R^{f}, wherein R^{a} and R^{f} may together along with the atom(s) to which they are attached form a 3-8 membered heterocyclic ring, which may contain a second heterogroup selected from O, NR^{e} or S];
Group VI is halogen, hydroxy, oxo, C₁-C₆alkoxy, aryl, heteroaryl, C₃-C₈cycloalkyl, heterocycloalkyl, —CN, or —OCF₃;
R^{e} is hydrogen, —CN, C₁-C₁₀alkyl, [C₁-C₁₀alkyl substituted with one to three substitutents independently selected from Group V], C₂-C₁₀alkenyl, C₂-C₁₀alkoxy, C₃-C₁₀cycloalkyl, aryl, heteroaryl, —C(═O)R^{f}, —C(═O)OR^{f}, —C(═O)NR^{a}R^{f}, —S(═O)₂NR^{a}R^{f}, or
—S(═O)₂R^{f};
R^{f} is hydrogen, C₁-C₁₀alkyl, [C₁-C₁₀alkyl substituted with from one to three substituents selected from Group VI], C₂-C₁₀alkenyl, C₂-C₁₀alkoxy, C₃-C₁₀cycloalkyl, heterocycloalkyl, aryl, or heteroaryl; and
R^{f} is hydrogen, C₁-C₆alkyl, C₃-C₈cycloalkyl, C₂-C₆ alkenyl, aryl, —C(═O)R^{f}, —C(═O)OR^{f}, —C(═O)NR^{a}R^{f}, or —S(═O)₂R^{f}, provided that R¹ and R² are not both hydrogen, further provided that when X is CH₂, W is NR^{a}, R³ is hydrogen and R⁵ is —OH, then R⁶ and R⁴ are not both —C(CH₃)₃, further provided that when X is CH₂ or CH₂CH₂, W is O, and R³ and R⁶ are hydrogen, then R⁴ is not halogen, —CF₃, C₁-C₆alkyl or C₃-C₇cycloalkyl, and further provided that when R³ and R⁴ are hydrogen and W is O then R⁶ is not halogen, —CF₃, C₁-C₆alkyl or C₃-C₇cycloalkyl.

10. A method for increasing the rate of nail growth in a mammal which comprises the administration to the mammal of an effective amount of a compound of the formula 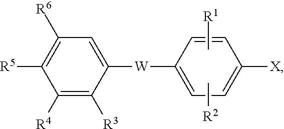
a stereoisomer or prodrug thereof, or a pharmaceutically acceptable salt of said compound, stereoisomer, or prodrug, wherein:
W is oxygen, sulfur, —SO—, —S(O)₂, —CH₂—, —CF₂—, —CHF—, —C(O) —, —CH(OH)—, —NR^{a}, or —C(═CH₂) —;
R¹, R², R³, and R⁶ are each independently hydrogen, halogen, —(C₁-C₈)alkyl, —CF₃, —OCF₃, —O(C₁-C₈)alkyl, or —CN;
R⁴ is hydrogen, —(C₁-C₁₂)alkyl substituted with zero to three substituents independently selected from Group V, —(C₂-C₁₂)alkenyl, —(C₂-C₁₂)alkynyl, halogen, —CN, —OR^{b}, —SR^{c}, —S(O)R^{c}, —S(O)₂R^{c}, aryl, heteroaryl, —(C₃-C₁₀)cycloalkyl, heterocycloalkyl, —S(O)₂NR^{c}R^{d}, —C(O)NR^{c}R^{d}, —C(O)OR^{c}, —NR^{a}C(O)R^{d}, —NR^{a}C(O)NR^{c}R^{d}, —NR^{a}S(O)₂R^{d}, or —C(O)R^{c}; or
R³ and R⁴ are taken together along with the carbon atoms to which they are attached to form a carbocyclic ring of formula —(CH₂)ᵢ— or a heterocyclic ring of formula —(CH₂)ₖ-Q-(CH₂)ₗ— wherein Q is oxygen, sulfur, or —NR^{e}—; i is 3, 4, 5, or 6; k is 0, 1, 2, 3, 4, or 5; and l is 0, 1, 2, 3, 4, or 5; and wherein said carbocyclic ring and said heterocyclic ring are each substituted with zero to four substituents independently selected from —(C₁-C₄)alkyl, —OR^{b}, oxo, —CN, phenyl, or —NR^{a}R^{g};
R⁵ is hydroxy, —O(C₁-C₆)alkyl, —OC(O)R^{f}, fluorine, or —C(O)OR^{c}; or
R⁴ and R⁵ are taken together along with the carbon atoms to which they are attached to form a heterocyclic ring selected from the group consisting of —CR^{c}═CR^{a}—NH—, —N═CR^{a}—NH, —CR^{c}═CR^{a}—O—, —CR^{a}═CR^{a}—S—, —CR^{c}═N—NH—, and —CR^{a}═CR^{a}—CR^{a}═N—;
R^{a} for each occurrence is independently hydrogen, or —(C₁-C₆)alkyl substituted with zero or one —(C₃-C₆)cycloalkyl or methoxy;
R^{b} for each occurrence is independently hydrogen, —(C₁-C₁₂)alkyl substituted with zero to three substituents independently selected from Group V, aryl, heteroaryl, —(C₃-C₁₀)cycloalkyl, heterocycloalkyl, —C(O)NR^{c}R^{d}, or —C(O)R^{f};
R^{c} and R^{d} for each occurrence are each independently hydrogen, —(C₁-C₁₂)alkyl substituted with zero to three substituents independently selected from Group VI, —(C₂-C₁₂)alkenyl, —(C₂-C₁₂)alkynyl, aryl, heteroaryl, —(C₃-C₁₀)cycloalkyl, or heterocycloalkyl;
provided that when R⁴ is the moiety —SR^{c}, —S(O)R^{c}, or —S(O)₂R^{c}, R^{c} is other than hydrogen; or
R^{c} and R^{d} are taken together along with the atom(s) to which they are attached to form a 3-10 membered heterocylic ring which may optionally contain a second heterogroup selected from oxygen, —NR^{e}—, or sulfur; and wherein said heterocyclic ring is substituted with zero to four substituents independently selected from —(C₁-C₄)alkyl, —OR^{b}, oxo, —CN, phenyl, or —NR^{a}R^{g};
R^{e} for each occurrence is hydrogen, —CN, —(C₁-C₁₀)alkyl substituted with zero to three substituents independently selected from Group V, —(C₂-C₁₀)alkenyl, —(C₂-C₁₀)alkoxy, —(C₃-C₁₀)cycloalkyl, aryl, heteroaryl, —C(O)R^{f}, —C(O)OR^{f}, —C(O)NR^{a}R^{f}, or —S(O)₂R^{f};
R^{f} for each occurrence is independently —(C₁-C₁₀)alkyl substituted with zero to three substituents independently selected from Group VI, —(C₂-C₁₂)alkenyl, —(C₂-C₁₀)alkynyl, —(C₃-C₁₀)cycloalkyl, aryl, heteroaryl, or heterocycloalkyl;
R^{g} for each occurrence is independently hydrogen, —(C₁-C₆)alkyl, —(C₂-C₆)alkenyl, aryl, —C(O)R^{f}, —C(O)OR^{f}, —C(O)NR^{a}R^{f}, —S(O)₂R^{f}, or —(C₃-C₈)cycloalkyl;
Group V is halogen, —CF₃, —OCF₃, —OH, oxo, —(C₁-C₆)alkoxy, —CN, aryl, heteroaryl, —(C₃-C₁₀)cycloalkyl, heterocycloalkyl, —SR^{f}, —S(O)R^{f}, —S(O)₂R^{f}, —S(O)₂NR^{a}R^{f}, —NR^{a}R^{g}, or —C(O)NR^{a}R^{f};
Group VI is halogen, hydroxy, oxo, —(C₁-C₆)alkoxy, aryl, heteroaryl, —(C₃-C₈)cycloalkyl, heterocycloalkyl, —CN, or —OCF₃;
provided that when R⁴ is —(C₁-C₁₂)alkyl substituted with zero to three substituents independently selected from Group V, wherein said Group V substituent is oxo, said oxo group is substituted on a carbon atom other than the C₁ carbon atom in —(C₁-C₁₂)alkyl;
aryl for each occurrence is independently phenyl or naphthyl substituted with zero to four substituents independently selected from halogen, —(C₁-C₆)alkyl, —CN, —SR^{f}, —S(O)R^{f}, —S(O)₂R^{f}, —(C₃-C₆)cycloalkyl, —S(O)₂NR^{a}R^{f}, —NR^{a}R^{g}, —C(O)NR^{a}R^{f}, —OR^{b}, -perfluoro-(C₁-C₄)alkyl, or —COOR^{f};
provided that when said substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, said substituents R^{b}, R^{f}, and R^{g}, are other than aryl or heteroaryl;
heteroaryl for each occurrence is independently a 5-, 6-, 7-, 8-, or 9-membered monocyclic or bicyclic ring having from one to three heteroatoms selected from O, N, or S;
wherein in said bicyclic ring, a monocyclic heteroaryl ring is fused to a benzene ring or to another heteroaryl ring, and having zero to three substituents independently selected from halogen, —(C₁-C₄)alkyl, —CF₃, —OR^{b}, —NR^{a}R^{g}, or —COOR^{f};
provided that when said substituent(s) on heteroaryl are —NR^{a}R^{g}, —OR^{b}, or —COOR^{f}, said substituents R^{b}, R^{f}, and R^{g}, are other than aryl or heteroaryl;
heterocycloalkyl for each occurrence is independently a 5-, 6-, 7-, 8-, or 9-membered monocyclic or bicyclic cycloalkyl ring having from one to three heteroatoms selected from oxygen, —NR^{e}, or sulfur, and having zero to four substituents independently selected from —(C₁-C₄)alkyl, —OR^{b}, oxo, —CN, phenyl, or —NR^{a}R^{g}; and
X is

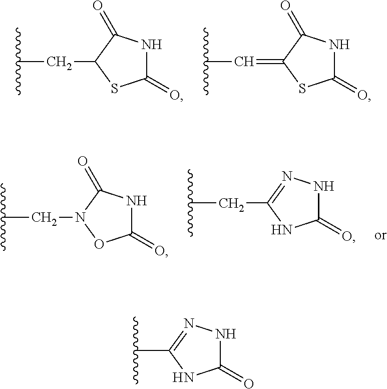

11. A method for increasing the rate of nail growth in a mammal which comprises the administration to the mammal of an effective amount of a compound of the formula 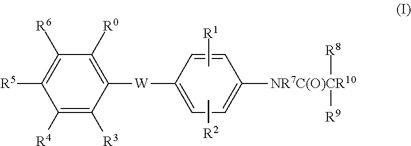
an isomer thereof, a prodrug of said compound or isomer, or a pharmaceutically acceptable salt of said compound, isomer or prodrug; wherein W is (a) —O—, (b) —S—, (c) —SO—, (d) —SO₂—, (e) —CH₂—, (f) —CF₂—, (g) —CHF—, (h) —C(O) —, (i) —CH(OH) —, (j) —NR^{a} or (k) 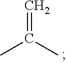
R⁰ is (a) hydrogen, (b) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (c) —C(O)R^{h}, (d) —S(O)₂R^{h} or (e) halogen;
R¹, R², R³ and R⁶ are each independently (a) hydrogen, (b) halogen, (c) —(C₁-C₈)alkyl, (d) —CF₃, (e) (f) —O(C₁-C₈)alkyl, or (g) —CN;
R⁴ is (a) hydrogen, (b) —(C₁-C₁₂)alkyl substituted with zero to three substituents independently selected from Group V, (c) —(C₂-C₁₂)alkenyl, (d) —(C₂-C₁₂)alkynyl, (e) halogen, (f) —CN, (g) —OR^{b}, (h) —SR^{c}, (i) —S(O)R^{c}, (j) —S(O)₂R^{c}, (k) aryl, (l) heteroaryl, (m) —(C₃-C₁₀)cycloalkyl, (n) heterocycloalkyl, (O) —S(O)₂NR^{c}R^{d}, (p) —C(O)NR^{c}R^{d}, (q) —C(O)OR^{c}, (r) —NR^{a}C(O)R^{d}, (s) —NR^{a}C(O)NR^{c}R^{d}, (t) —NR^{a}S(O)₂R^{d}, (u) —NR^{a}R^{d} or (v) —C(O)R^{c};
or R³ and R⁴ are taken together along with the carbon atoms to which they are attached to form a carbocyclic ring of formula —(CH₂)ᵢ— or a heterocyclic ring of formula —(CH₂)ₖ-Q-(CH₂)ₗ— wherein Q is —O—, —S— or —NR^{e}—; i is 3, 4, 5 or 6; k is 0, 1, 2, 3, 4 or 5; and l is 0, 1, 2, 3, 4 or 5; and wherein the carbocyclic ring and the heterocyclic ring are each substituted with zero to four substituents independently selected from (a) —(C₁-C₄)alkyl, (b) —OR^{b}, (c) oxo, (d) —CN, (e) phenyl or (f) —NR^{a}R^{g};
R⁵ is (a) —OH, (b) —O(C₁-C₆)alkyl, (c) —OC(O)R^{f}, (d) F, or (e) —C(O)OR^{c};
or R⁴ and R⁵ are taken together along with the carbon atoms to which they are attached to form a heterocyclic ring selected from the group consisting of —CR^{c}═CR^{a}—NH—, —N═CR^{a}—NH, —CR^{c}═CR^{a}—O—, —CR^{c}═CR^{a}—S—, —CR^{c}═N—NH— and —CR^{a}═CR^{a}—CR^{a}═N—;
R⁷ is (a) hydrogen or (b) —(C₁-C₆)alkyl;
R⁸ and R⁹ are each independently (a) hydrogen, (b) —(C₁-C₆)alkyl, (c) aryl, or (d) halogen;
R¹⁰ is (a) —(C₀-C₁)alkyl-C(O)OH, (b) —(C₀-C₁)alkyl-C(O)OR^{f}, (c) —(C₀-C₁)alkyl-C(O)NR^{c}R^{d}, or (d) —(C₀-C₁)alkyl-OH;
R^{a} for each occurrence is independently (a) hydrogen or (b) —(C₁-C₆)alkyl substituted with zero or one —(C₃-C₆)cycloalkyl or methoxy;
R^{b} for each occurrence is independently (a) hydrogen, (b) —(C₁-C₁₂)alkyl substituted with zero to three substituents independently selected from Group V, (c) aryl, (d) heteroaryl, (e) —(C₃-C₁₀)cycloalkyl, (f) heterocycloalkyl, (g) —C(O)NR^{c}R^{d}, or (h) —C(O)R^{f};
R^{c} and R^{d} for each occurrence are each independently (a) hydrogen, (b) —(C₁-C₁₂)alkyl substituted with zero to three substituents independently selected from Group VI, (c) —(C₂-C₁₂)alkenyl, (d) —(C₂-C₁₂)alkynyl, (e) aryl, (f) heteroaryl, (g) —(C₃-C₁₀)cycloalkyl or (h) heterocycloalkyl;
provided that when R⁴ is the moiety —SR^{C}, —S(O)R^{c} or —S(O)₂R^{c}, R^{c} is other than hydrogen;
or R^{c} and R^{d} are taken together along with the atom(s) to which they are attached to form a 3-10 membered heterocyclic ring which may optionally contain a second heterogroup selected from —O—, —NR^{e}— or —S—; and wherein the heterocyclic ring is substituted with zero to four substituents independently selected from (a) —(C₁-C₄)alkyl, (b) —OR^{b}, (c) oxo, (d) —CN, (e) phenyl or (f) —NR^{a}R^{g};
R^{e} for each occurrence is (a) hydrogen, (b) —CN, (c) —(C₁-C₁₀)alkyl substituted with zero to three substitutents independently selected from Group V, (d) —(C₂-C₁₀)alkenyl, (e) —(C₂-C₁₀)alkoxy, (f) —(C₃-C₁₀)cycloalkyl, (g) aryl, (h) heteroaryl, (i) —C(O)R^{f}, (j) —C(O)OR^{f}, (k) —C(O)NR^{a}R^{f} or (l) —S(O)₂R^{f};
R^{f} for each occurrence is independently (a) —(C₁-C₁₀)alkyl substituted with zero to three substituents independently selected from the Group VI, (b) —(C₂-C₁₀)alkenyl, (c) —(C₂-C₁₀)alkynyl, (d) —(C₃-C₁₀)cycloalkyl, (e) aryl, (f) heteroaryl or (g) heterocycloalkyl;
R^{g} for each occurrence is independently (a) hydrogen, (b) —(C₁-C₆)alkyl, (c) —(C₂-C₆)alkenyl, (d) aryl, (e) —C(O)R^{f}, (f) —C(O)OR^{f}, (g) —C(O)NR^{a}R^{f}, (h) —S(O)₂R^{f} or (i) —(C₃-C₆)cycloalkyl;
R^{h} is (a) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (b) phenyl substituted with zero to two substituents independently selected from the group consisting of (1) —(C₁-C₄)alkyl, (2) halogen, (3) —CF₃ and (4) —OCF₃; (c) —(C₃-C₆)cycloalkyl or (d) heterocycloalkyl;
Group V is (a) halogen, (b) —CF₃, (c) —OCF₃, (d) —OH, (e) -oxo, (f) —(C₁-C₆)alkoxy, (g) —CN, (h) aryl, (i) heteroaryl, a) —(C₃-C₁₀)cycloalkyl, (k) heterocycloalkyl, (l) —SR^{f}, (m) —S(O)R^{f}, (n) —S(O)₂R^{f}, (O) —S(O)₂NR^{a}R^{f} (p) —NR^{a}R^{g} or (q) —C(O)NR^{a}R^{f};
Group VI is (a) halogen, (b) hydroxy, (c) oxo, (d) —(C₁-C₆)alkoxy, (e) aryl, (f) heteroaryl, (g) —(C₃-C₈)cycloalkyl, (h) heterocycloalkyl, (i) —CN, or (j) —OCF₃;
provided that when the substituent R⁴ is —(C₁-C₁₂)alkyl substituted with zero to three substituents independently selected from Group V wherein the Group V substituent is oxo, the oxo group is substituted on a carbon atom other than the C₁ carbon atom in —(C₁-C₁₂)alkyl;
aryl for each occurrence is independently phenyl or naphthyl substituted with zero to four substituents independently selected from (a) halogen, (b) —(C₁-C₆)alkyl, (c) —CN, (d) —SR^{f}, (e) —S(O)R^{f}, (f) —S(O)₂R^{f}, (g) —(C₃-C₆)cycloalkyl, (h) —S(O)₂NR^{a}R^{f}, (i) —NR^{a}R^{g}, (j) —C(O)NR^{a}R^{f}, (k) —OR^{b}, (l) -perfluoro-(C₁-C₄)alkyl, or (m) —COOR^{f};
provided that when the substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, the substituents R^{b}, R^{f} and R^{g} are other than aryl or heteroaryl;
heteroaryl for each occurrence is independently a 5-, 6-, 7-, 8-, 9- or 10-membered monocyclic or bicyclic ring having from 1 to 3 heteroatoms selected from O, N or S; wherein in the bicyclic ring, a monocyclic heteroaryl ring is fused to a benzene ring or to another heteroaryl ring; and having zero to three substituents independently selected from (a) halogen, (b) —(C₁-C₄)alkyl, (c) —CF₃, (d) —OR^{b}, (e) —NR^{a}R^{g}, or (f) —CO₂R^{f};
provided that when the substituent(s) on heteroaryl are —OR^{b}, —NR^{a}R^{g} or —CO₂R^{f}, the substituents R^{b}, R^{f} and R^{g} are other than aryl or heteroaryl;
heterocycloalkyl for each occurrence is independently a 4-, 5-, 6-, 7-, 8-, 9- or 10-membered monocyclic or bicyclic cycloalkyl ring having from 1 to 3 heteroatoms selected from O, NR^{e} or S; and having zero to four substituents independently selected from (a) —(C₁-C₄)alkyl, (b) —OR^{b}, (c) oxo, (d) —CN, (e) phenyl or (f) —NR^{a}R^{g}.

12. A method of claim 11 which comprises administering a compound selected from the group consisting of:
N-{4-[3-(cyclobutyl-methyl-carbamoyl)-4-hydroxy-phenoxy]-3,5-dimethyl-phenyl)}-malonamic acid;
N-{3-chloro-4-[4-hydroxy-3-(1-isopropyl-2-methyl-propylcarbamoyl)-phenoxy]-5-methyl-phenyl}-malonamic acid;
N-{3,5-dichloro-4-[3-((1S)-cyclohexyl-ethylcarbamoyl)-4-hydroxy-phenoxy]-phenyl}-malonamic acid;
N-[3,5-dichloro-4-(3-cyclopropylsulfamoyl-4-hydroxy-phenoxy)-phenyl]-malonamic acid;
N-[3,5-dichloro-4-(3-cyclobutylsulfamoyl-4-hydroxy-phenoxy)-phenyl]-malonamic acid;
N-[3-chloro-4-(3-cyclobutylsulfamoyl-4-hydroxy-phenoxy)-5-methyl-phenyl]-malonamic acid;
N-[4-(3-cyclobutylsulfamoyl-4-hydroxy-phenoxy)-3,5-dimethyl-phenyl]-malonamic acid;
N-[4-(3-cyclopropylsulfamoyl-4-hydroxy-phenoxy)-3,5-dimethyl-phenyl]-malonamic acid;
N-[3-chloro-4-(3-cyclobutylmethanesulfonyl-4-hydroxy-phenoxy)-5-methyl-phenyl]-malonamic acid;
N-[3-chloro-4-(3-cyclopropylmethanesulfonyl-4-hydroxy-phenoxy)-5-methyl-phenyl]-malonamic acid;
N-[3,5-dichloro-4-(3-cyclopropylmethanesulfonyl-4-hydroxy-phenoxy)-phenyl]-malonamic acid;
N-[4-(3-cyclobutylmethanesulfonyl-4-hydroxy-phenoxy)-3,5-dimethyl-phenyl]-malonamic acid;
N-[3,5-dichloro-4-(3-cyclobutylmethanesulfonyl-4-hydroxy-phenoxy)-phenyl]-malonamic acid;
N-[4-(3-cyclopentylmethanesulfonyl-4-hydroxy-phenoxy)-3,5-dimethyl-phenyl]-malonamic acid;
N-[4-(3-cyclobutylmethanesulfonyl-4-hydroxy-phenoxy)-3,5-dimethyl-phenyl]-2-methyl-malonamic acid;
N-[3-chloro-4-(3-cyclohexylmethanesulfonyl-4-hydroxy-phenoxy)-5-methyl-phenyl]-malonamic acid;
N-[3-chloro-4-(3-cyclobutylmethanesulfonyl-4-hydroxy-phenoxy)-5-methyl-phenyl]-2-methyl-malonamic acid;
N-[3-chloro-4-(3-cyclopentylmethanesulfonyl-4-hydroxy-phenoxy)-5-methyl-phenyl]-malonamic acid;
N-[4-(3-cyclohexylmethanesulfonyl-4-hydroxy-phenoxy)-3,5-dimethyl-phenyl]-malonamic acid;
N-{4-[3-(4-fluoro-benzenesulfonyl)-4-hydroxy-phenoxy]-3,5-dimethyl-phenyl}-malonamic acid;
N-{3-chloro-4-[3-(4-fluoro-benzenesulfonyl)-4-hydroxy-phenoxy]-5-methyl-phenyl}-malonamic acid;
N-{4-[3-(4-fluoro-benzoyl)-4-hydroxy-phenoxy]-3,5-dimethyl-phenyl}-malonamic acid;
N-[4-(3-cyclopentylacetyl-4-hydroxy-phenoxy)-3,5-dimethyl-phenyl]-malonamic acid;
N-(4-{3-[(4-fluoro-phenyl)-hydroxy-methyl]-4-hydroxy-phenoxy}-3,5-dimethyl-phenyl)-malonamic acid;
N-{4-[3-(2-cyclopentyl-1-hydroxy-ethyl)-4-hydroxy-phenoxy]-3,5-dimethyl-phenyl}-malonamic acid;
N-[3-chloro-4-(3-cyclobutylmethanesulfonyl-4-hydroxy-phenoxy)-5-methyl-phenyl]-malonamic acid methyl ester;
N-[3-chloro-4-(3-cyclobutylmethanesulfonyl-4-hydroxy-phenoxy)-5-methyl-phenyl]-malonamic acid ethyl ester;
N-[4-(3-cyclobutylmethanesulfonyl-4-hydroxy-phenoxy)-3,5-dimethyl-phenyl]-malonamic acid ethyl ester;
N-[4-(3-cyclobutylmethanesulfonyl-4-hydroxy-phenoxy)-3,5-dimethyl-phenyl]-malonamic acid methyl ester;
N-[3-chloro-4-(3-cyclopentanesulfonyl-4-hydroxy-phenoxy)-5-methyl-phenyl]-malonamic acid;
N-[4-(3-cyclopentanesulfonyl-4-hydroxy-phenoxy)-3,5-dimethyl-phenyl]-malonamic acid;
N-{3-chloro-4-[3-(4-fluoro-benzenesulfonyl)-4-hydroxy-phenoxy]-5-methyl-phenyl}-malonamic acid;
N-{3-chloro-4-[3-(4-fluoro-benzenesulfonyl)-4-hydroxy-phenoxy]-5-methyl-phenyl}-malonamic acid methyl ester;
N-{3,5-dichloro-4-[3-(4-fluoro-benzenesulfonyl)-4-hydroxy-phenoxy]-phenyl}-malonamic acid methyl ester;
N-{3,5-dichloro-4-[3-(4-fluoro-benzenesulfonyl)-4-hydroxy-phenoxy]-phenyl}-malonamic acid ethyl ester;
N-{4-[3-(4-fluoro-benzenesulfonyl)-4-hydroxy-phenoxy]-3,5-dimethyl-phenyl}-2-methyl-malonamic acid methyl ester;
N-{4-[3-(4-fluoro-benzenesulfonyl)-4-hydroxy-phenoxy]-3,5-dimethyl-phenyl}-2-methyl-malonamic acid;
N-{3-chloro-4-[3-(4-fluoro-benzenesulfonyl)-4-hydroxy-phenoxy]-5-methyl-phenyl}-2-methyl-malonamic acid methyl ester;
N-{3-chloro-4-[3-(4-fluoro-benzenesulfonyl)-4-hydroxy-phenoxy]-5-methyl-phenyl}-2-methyl-malonamic acid;
N-{3,5-dichloro-4-[3-(4-fluoro-benzenesulfonyl)-4-hydroxy-phenoxy]-phenyl}-2-methyl-malonamic acid methyl ester; and
N-{3,5-dichloro-4-[3-(4-fluoro-benzenesulfonyl)-4-hydroxy-phenoxy]-phenyl}-2-methyl-malonamic acid;
N-{4-[3-(4-fluoro-benzenesulfonyl)-4-hydroxy-phenoxy]-3,5-dimethyl-phenyl}-malonamic acid; and
N-{4-[3-(4-fluoro-benzenesulfonyl)-4-hydroxy-phenoxy]-3,5-dimethyl-phenyl}-malonamic acid methyl ester; or an isomer thereof, a prodrug of said compound or isomer, or a pharmaceutically acceptable salt of said compound, isomer or prodrug.

13. A method for increasing the rate of nail growth in a mammal which comprises the administration to the mammal of an effective amount of a compound of the formula 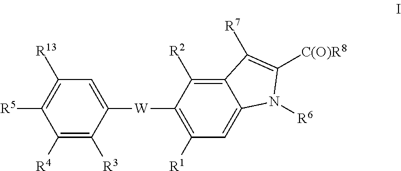
or the pharmaceutically acceptable salt thereof; wherein
W is oxygen, CH₂, CF₂, NR¹², S(O)ₘ wherein m is 0, 1 or 2;
R¹, R², and R³ are each independently selected from the group consisting of hydrogen, halo, cyano, trifluoromethyl, trifluomethoxy and (C₁-C₆)alkyl;
R⁴ is hydrogen, halo, cyano, (C₁-C₁₂)alkyl, (C₂-C₁₂)alkenyl, (C₂-C₁₂)alkynyl, (C₃-C₁₀)cycloalkyl, (C₃-C₁₀)cycloalkyl(C₁-C₆)alkyl, (C₆-C₁₀)aryl, (C₆-C₁₀)aryl(C₁-C₆)alkyl, (C₂-C₉)heteroaryl, (C₂-C₉)heteroaryl(C₁-C₆)alkyl, (C₂-C₉)heterocycloalkyl, (C₂-C₉)heterocycloalkyl(C₁-C₆)alkyl, —OR⁹, —S(O)₂NR¹⁰R¹¹, —C(O)NR¹⁰R¹¹, —C(O)R¹⁰, —CH(OH)R¹⁰, —NR¹²C(O)R¹⁰, —NR¹²C(O)NR¹⁶R¹¹, —NR¹²S(O)₂R¹⁰ or —S(O)ₙR¹⁰ wherein n is 0, 1 or 2;
R⁵ is hydroxy, fluoro, (C₁-C₄)alkoxy or —OC(O)R¹⁰;
R⁶ is hydrogen, —C(O)CH₃ or (C₁-C₆)alkyl;
R⁷ is hydrogen or (C₁-C₆)alkyl;
R⁸ is OR¹² or NR⁹R¹²;
R⁹ for each occurrence is independently hydrogen, (C₁-C₁₂)alkyl, (C₃-C₁₀)cycloalkyl, (C₂-C₉)heterocycloalkyl, (C₆-C₁₀)aryl or (C₂-C₉)heteroaryl;
R¹⁰ for each occurrence is independently hydrogen, (C₁-C₁₂)alkyl, (C₂-C₁₂)alkenyl, (C₂-C₁₂)alkynyl, (C₃-C₁₀)cycloalkyl, (C₃-C₉)cycloalkyl(C₁-C₆)alkyl, (C₆-C₁₀)aryl, (C₂-C₉)heteroaryl or halo(C₆-C₁₀)aryl;
R¹¹ for each occurrence is independently hydrogen, (C₁-C₆)alkyl, (C₃-C₁₀) cycloalkyl or (C₃-C₉)cycloalkyl(C₁-C₆)alkyl;
or R¹⁰ and R¹¹ may be taken together with the nitrogen to which they are attached to form a 3 to 10 membered heterocyclic group which may contain a second heteroatom selected from oxygen, sulfur or NR¹⁴ wherein R¹⁴ is hydrogen or (C₁-C₆)alkyl;
R¹² for each occurrence is independently hydrogen or (C₁-C₆)alkyl;
R¹³ is hydrogen, halo or (C₁-C₆)alkyl;
or R³ and R⁴ may be taken together with the carbons to which they are attached to form a compound of the formula 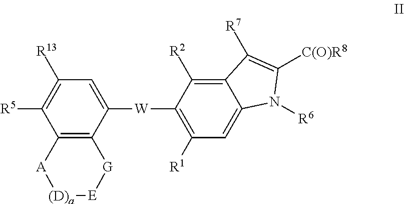
wherein a is 0, 1, 2 or 3;
A, D, E and G are each independently selected from the group consisting of CR¹⁶R¹⁷, NR¹⁸, oxygen or sulfur;
R¹⁶ and R¹⁷ for each occurrence are each independently selected from hydrogen or (C₁-C₆)alkyl; and
R¹⁸ is hydrogen, (C₁-C₆)alkyl, —C(O)R¹⁰ or —S(O)₂R¹⁰ wherein R¹⁰ is defined as above.

14. A method of claim 13 wherein the compound is selected from the group consisting of:
5-(4-Hydroxy-3-isopropyl-phenoxy)-4,6-dimethyl-1H-indole-2-carboxylic acid;
4,6-Dichloro-5-(4-hydroxy-3-isopropyl-phenoxy)-1H-indole-2-carboxylic acid;
5-(3-sec-butyl-4-hydroxy-phenoxy)-4,6-dimethyl-1H-indole-2-carboxylic acid;
5-[3-(4-Fluoro-benzyl)-4-hydroxy-phenoxyl]-4,6-dimethyl-1H-indole-2-carboxylic acid;
5-{3-[(4-Fluoro-phenyl)-hydroxy-methyl]-4-hydroxy-phenoxy}-4,6-dimethyl-1H-ondole-2-carboxylic acid;
5-[3-(2-Cyclopentyl-1-hydroxy-ethyl)-4-hydroxy-phenoxyl]-4,6-dimethyl-1H-indole-2-carboxylic acid;
5-[3-(4-Fluoro-benzoyl)-4-hydroxy-phenoxyl]-4,6-dimethyl-1H-indole-2-carboxylic acid;
5-[3-(Cyclobutyl-methyl-carbamoyl)-4-hydroxy-phenoxyl]-4,6-dim ethyl-1H-indole-2-carboxylic acid;
5-(3-Cyclobutylmethanesulfonyl-4-hydroxy-phenoxy)-4,6-dimethyl-1H-indole-2-carboxylic acid;
4,6-Dichloro-5-(3-cyclobutylmethanesulfonyl-4-hydroxy-phenoxy)-1H-indole-2-carboxylic acid;
4,6-Dichloro-5-(4-hydroxy-3-isopropyl-phenoxy)-1-methyl-1H-indole-2-carboxylic acid;
4,6-Dichloro-5-(4-hydroxy-3-isopropyl-phenoxy)-3-methyl-1H-indole-2-carboxylic acid;
5-[3-(4-Fluoro-benzenesulfonyl)-4-hydroxy-phenoxy]-4,6-dimethyl-1H-indole-2-carboxylic acid;
5-[3-(4-Fluoro-benzenesulfonyl)-4-hydroxy-phenoxy]-3,4,6-trimethyl-1H-indole-2-carboxylic acid;
4,6-Dichloro-5-(3-cyclobutylsulfamoyl-4-hydroxy-phenoxy)-1H-indole-2-carboxylic acid;
4-Chloro-50(3-cyclopropylsulfamoyl-4-hydroxy-phenoxy)-5-methyl-1H-indole-2-carboxylic acid;
4,6-Dichloro-5-[4-hydroxy-3-(1-isopropyl-2-methyl-propylcarbamoyl)-phenoxy]-1H-indole-2-carboxylic acid;
5-[3-(4-Fluoro-benzenesulfonyl)-4-hydroxy-phenoxy]-1,4,6-trimethyl-1H-indole-2-carboxylic acid;
5-{3-[(4(4-Fluoro-phenyl)-hydroxy-methyl]-4-hydroxy-phenoxy)-3,4,6-trimethyl-1H-indole-2-carboxylic acid;
5-[3-(4-Fluoro-benzyl)-4-hydroxy-phenoxy]-3,4,6-trimethyl-1H-indole-2-carboxylic acid;
5-(3-Cyclopentylmethanesulfonyl-4-hydroxy-phenoxy)-4,6-dimethyl-1H-indole-2-carboxylic acid;
4,6-Dichloro-5-(3-cyclopropylsulfamoyl-4-hydroxy-phenoxy)-1H-indole-2-carboxylic acid;
5-(3-Cyclohexylmethanesulfonyl-4-hydroxy-phenoxy)-4,6-dimethyl-1H-indole-2-carboxylic acid;
5-(3-Cyclopropylsulfamoyl-4-hydroxy-phenoxy)-4,6-dimethyl-1H-indole-2-carboxylic acid;
5-(4-Hydroxy-3-isopropyl-phenoxy)-3,4,6-trimethyl-1H-indole-2-carboxylic acid;
5-(4-Hydroxy-3-isopropyl-phenoxy)-1,4,6-trimethyl-1H-indole-2-carboxylic acid;
4,6-Dichloro-5-[3-(4-fluoro-benzenesulfonyl)-4-hydroxy-phenoxy]-1H-indole-2-carboxylic acid;
4,6-Dichloro-5-[3-(4-fluoro-benzenesulfonyl-4-hydroxy-phenoxy]-3-methyl-1H-indole-2-carboxylic acid;
5-(3-Cyclobutylsulfamoyl-4-hydroxy-phenoxy)-4,6-dimethyl-1H-indole-2-carboxylic acid;
5-[4-Hydroxy-3-(1-isopropyl-2-methyl-propylcarbamoyl)-phenoxy]-4,6-dimethyl-1H-indole-2-carboxylic acid;
4,6-Dichloro-5-(3-[(4-fluoro-phenyl)-hydroxy-methyl]-4-hydroxy-phenoxy)-1H-indole-2-carboxylic acid;
5-(4-Hydroxy-2,3-dimethyl-phenoxy)-4,6-dimethyl-1H-indole-2-carboxylic acid;
4,6-Dichloro-5-(4-hydroxy-2,3-dimethyl-phenoxy)-1H-indole-2-carboxylic acid;
5-(7-Hydroxy-indan-4-yloxy)-4,6-dimethyl-1H-indole-2-carboxylic acid;
4,6-Dichloro-5-(7-hydroxy-indan-4-yloxy)-1H-indole-2-carboxylic acid;
4,6-Dichloro-5-(4-hydroxy-5,6,7,8-tetrahydro-naphthalen-1-yloxy)-1H-indole-2-carboxylic acid; and
5-(4-Hydroxy-5,6,7,8-tetrahydro-naphthalen-1-yloxy)-4,6-dimethyl-1H-indole-2-carboxylic acid.

15. A method of claim 1, 4, 6, 7, 9, 10, 11 or 13 wherein the compound is cardiac-sparing.

16. A method of claim 1, 4, 6, 7, 9, 10, 11 or 13 wherein the mammal is a human being.

17. A method of claim 1, 4, 6, 7, 9, 10, 11 or 13 wherein the administration is topical.

18. A method of claim 1, 4, 6, 7, 9, 10, 11 or 13 wherein the effective amount of the compound is about 0.0001% to about 10% (w/v) of the compound per day.

19. A method of claim 1, 4, 6, 7, 9, 10, 11 or 13 which comprises the administration of an effective amount of a nail growth promoter, an antibacterial agent or an antifungal agent.

20. A method of claim 1, 4, 6, 7, 9, 10, 11 or 13 wherein the antifungal agent is fluconazole, itraconazole, terbinafine or ciclopirox.

21. A topical pharmaceutical composition for increasing the rate of nail growth which comprises an effective amount of a compound of the formula 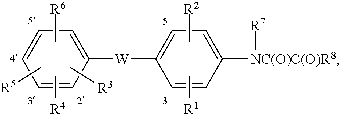
a prodrug thereof, a geometric or optical isomer thereof, or a pharmaceutically acceptable salt of said compound, said prodrug, or said isomer, wherein:
R¹, R² and R³ are each independently hydrogen, halogen, C₁₋₆ alkyl, trifluoromethyl, —CN, —OCF₃ or —OC₁₋₆ alkyl;
R⁴ is hydrogen, C₁₋₁₂ alkyl optionally substituted with one to three substitutents independently selected from Group Z, C₂₋₁₂ alkenyl, halogen, —CN, aryl, heteroaryl, C₃₋₁₀ cycloalkyl, heterocycloalkyl, —S(O)₂NR⁹R¹⁰, —C(O)NR⁹R¹⁰, —(C₁₋₆ alkyl) —NR⁹R¹⁰, —NR⁹C(O)R¹⁰, —NR⁹C(O)NR⁹R¹⁰, —NR⁹S(O)₂R¹⁰, —(C₁₋₆ alkyl) —OR¹¹, —OR¹¹ or —S(O)ₐR¹², provided that, where R⁵ is not fluoro, R⁴ is —S(O)₂NR⁹R¹⁰, —C(O)NR⁹R¹⁰, —(C₁₋₆ alkyl) —NR⁹R¹⁰, —NR⁹C(O)R¹⁰, —NR⁹C(O)NR⁹R¹⁰, —NR⁹S(O)₂R¹⁰, —(C₁₋₆alkyl) —OR¹¹, —OR¹¹ or —S(O)ₐR¹²;
or R³ and R⁴ may be taken together to form a carbocyclic ring A of the formula —(CH₂)_{b}— or a heterocyclic ring A selected from the group consisting of -Q-(CH₂)_{c}— and —(CH₂)ⱼ-Q-(CH₂)ₖ— wherein Q is O, S or NR¹⁷, wherein said carbocyclic ring A and said heterocyclic ring A are each independently optionally substituted with one or more substituents independently selected from C₁₋₄ alkyl, halide or oxo;
R⁵ is fluoro, hydroxy, C₁₋₄ alkoxy or OC(O)R⁹;
or R⁴ and R⁵ may be taken together to form a heterocyclic ring B selected from the group consisting of —CR⁹═CR¹⁰—NH—, —N═CR⁹—NH—, —CR⁹═CH—O— and —CR⁹═CH—S—;
R⁶ is hydrogen, halogen, C₁₋₄ alkyl or trifluoromethyl;
R⁷ is hydrogen or C₁₋₆ alkyl;
R⁸ is —OR⁹ or —NR¹⁹R²³;
R⁹ and R¹⁹ for each occurrence are independently (A) hydrogen, (B) C₁₋₁₂ alkyl optionally substituted with one or more substituents independently selected from Group V, (C)C₂₋₁₂ alkenyl, (D) C₃₋₁₀ cycloalkyl optionally substituted with one or more substituents independently selected from C₁₋₆ alkyl, C₂₋₅ alkynyl, C₃₋₁₀ cycloalkyl, —CN, —NR¹³R¹⁴, oxo, —OR¹⁸, —COOR¹⁸ or aryl optionally substituted with X and Y, (E) aryl optionally substituted with X and Y, or (F) het optionally substituted with X and Y;
or R⁹ and R¹⁰ for any occurrence may be taken together to form a heterocyclic ring C optionally further containing a second heterogroup selected from the group consisting of —O—, —NR¹³— and —S—, and optionally further substituted with one or more substituents independently selected from C₁₋₅ alkyl, oxo, —NR¹³R¹⁴, —OR¹⁸, —C(O)₂R¹⁸, —CN, —C(O)R⁹, aryl optionally substituted with X and Y, het optionally substituted with X and Y, C₅₋₆ spirocycloalkyl, and a carbocyclic ring B selected from the group consisting of 5-, 6-, 7- and 8-membered partially and fully saturated, and unsaturated carbocyclic rings, and including any bicyclic group in which said carbocyclic ring B is fused to a carbocyclic ring C selected from the group consisting of 5-, 6-, 7- and 8-membered partially and fully saturated, and unsaturated carbocyclic rings;
R¹¹ is C₁₋₁₂ alkyl optionally substituted with one or more substituents independently selected from Group V, C₂₋₁₂ alkenyl, C₃₋₁₀ cycloalkyl, trifluoromethyl, difluoromethyl, monofluoromethyl, aryl optionally substituted with X and Y, het optionally substituted with X and Y, —C(O)NR⁹R¹⁰ or —C(O)R⁹;
R¹² is C₁₋₁₂ alkyl optionally substituted with one or more substituents independently selected from Group V, C₂₋₁₂ alkenyl, C₃₋₁₀ cycloalkyl, aryl optionally substituted with X and Y, or het optionally substituted with X and Y;
R¹³ and R¹⁴ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁵R¹⁶ or C₃₋₁₀ cycloalkyl;
R¹⁵ and R¹⁶ for each occurrence are independently hydrogen, C₁₋₆ alkyl, C₃-C₁₀cycloalkyl or aryl optionally substituted with X and Y;
R¹⁷ is hydrogen, C₁₋₆ alkyl, —COR⁹ or —SO₂R⁹;
R¹⁸ is hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁸R¹⁶, —(C₁₋₄ alkyl) —(C₁₋₄ alkoxy) or C₃₋₁₀ cycloalkyl;
R¹⁹ is hydrogen or C₁₋₆ alkyl;
R²⁰ is hydrogen or C₁₋₆ alkyl;
W is O, S(O)_{d}, CH₂ or NR⁹;
Group Z is C₂₋₆ alkenyl, C₂₋₆ alkynyl, halogen, —CF₃, —OCF₃, hydroxy, oxo, —CN, aryl, heteroaryl, C₃₋₁₀ cycloalkyl, heterocycloalkyl, —S(O)ₐR¹², —S(O)₂NR⁹R¹⁰, —C(O)R⁹R¹⁰, and —NR⁹R¹⁰;
Group V is halogen, —NR¹³R¹⁴, —OCF₃, —OR⁹, oxo, trifluoromethyl, —CN, C₃₋₁₀ cycloalkyl, aryl optionally substituted with X and Y, and het optionally substituted with X and Y;
het for each occurrence is a heterocyclic ring D selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring D is fused to a benzene ring or a heterocyclic ring E selected from the group consisting of 4-, 5-, 6-, 7- and 8-membered partially and fully saturated, and unsaturated, heterocyclic rings containing from one to four heteroatoms independently selected from the group consisting of N, O and S;
X and Y for each occurrence are independently (A) hydrogen, (B) halogen, (C) trifluoromethyl, (D) —OCF₃, (E) —CN, (F) C₁₋₆ alkyl optionally substituted with one or more substituents independently selected from the group consisting of halogen, —OCF₃, —CF₃ and phenyl, (G) C₁₋₆ alkoxy, (H) aryl optionally substituted with one or more substituents independently selected from the group consisting of halogen, —OCF₃, —CF₃, C₁₋₄ alkyl and C₁₋₄ alkoxy, (I) —C(O)₂R¹³, (J) —C(O)NR¹³R¹⁴, (K) —C(O)R¹³, (L) —NR¹³C(O)NR¹³R¹⁴ and (M) —NR¹³C(O)R¹⁴;
or X and Y for any occurrence in the same variable may be taken together to form (a) a carbocyclic ring D of the formula —(CH₂)ₑ— or (b) a heterocyclic ring F selected from the group consisting of —O(CH₂)_{f}O—, (CH₂)_{g}NH— and —CH═CHNH—;
a and d are each independently 0, 1 or 2;
b is 3, 4, 5, 6 or 7;
c, f, g, j and k are each independently 2, 3, 4, 5 or 6; and
e is 3, 4, 5, 6 or 7;
and a pharmaceutically acceptable carrier.

22. A composition of claim 21 wherein the compound is selected from the group consisting of:
N-[3-chloro-4-(3-cyclopropylsulfamoyl-4-hydroxy-phenoxy)-5-methyl-phenyl]-oxamic acid;
N-[4-(3-cyclopropylsulfamoyl-4-hydroxy-phenoxy)-3,5-dimethyl-phenyl]-oxamic acid;
N-{4-[3-(cyclobutyl-methyl-carbamoyl)-4-hydroxy-phenoxy]-3,5-dimethyl-phenyl}-oxamic acid;
N-{3-chloro-4-[3-(cyclobutyl-methyl-carbamoyl)-4-hydroxy-phenoxy]-5-methyl-phenyl}-oxamic acid;
N-[4-(7-hydroxy-indan-4-yloxy)-3,5-dimethyl-phenyl]oxamic acid;
N-{3,5-dichloro-4-[3-(cyclobutyl-methyl-carbamoyl)-4-hydroxy-phenoxy]-phenyl}-oxamic acid;
N-[3,5-dichloro-4-(3-cyclopentanesulfonyl-4-hydroxy-phenoxy)-phenyl]-oxamic acid;
N-[3,5-dichloro-4-(3-cyclopropylmethanesulfonyl-4-hydroxy-phenoxy)-phenyl]-oxamic acid;
N-[3,5-dichloro-4-(3-cyclobutylmethanesulfonyl-4-hydroxy-phenoxy)-phenyl]-oxamic acid;
N-[4-(3-cyclopropylmethanesulfonyl-4-hydroxy-phenoxy)-3,5-dimethyl-phenyl]-oxamic acid;
N-[3-chloro-4-(3-cyclobutylmethanesulfonyl-4-hydroxy-phenoxy)-5-methyl-phenyl]-oxamic acid;
N-[4-(3-cyclobutylmethanesulfonyl-4-hydroxy-phenoxy)-3,5-dimethyl-phenyl]-oxamic acid;
N-[4-(3-cyclopentylmethanesulfonyl-4-hydroxy-phenoxy)-3,5-dimethyl-phenyl]-oxamic acid;
N-[4-(3-chloro-4-(3-cyclopentylmethanesulfonyl-4-hydroxy-phenoxy)-5-methyl-phenyl]-oxamic acid;
N-[3,5-dichloro-4-(3-cyclopentylmethanesulfonyl-4-hydroxy-phenoxy)-phenyl]oxamic acid;
N-[4-(3-cyclohexylmethanesulfonyl-4-hydroxy-phenoxy)-3,5-dimethyl-phenyl]-oxamic acid;
N-[3-chloro-4-(3-cyclohexylmethanesulfonyl-4-hydroxy-phenoxy)-5-methyl-phenyl]-oxamic acid;
N-[3,5-dichloro-4-(3-cyclohexylmethanesulfonyl-4-hydroxy-phenoxy)-phenyl]-oxamic acid;
N-[3,5-dichloro-4-[3-(4-fluoro-benzenesulfonyl)-4-hydroxy-phenoxy)-phenyl]-oxamic acid;
N-{4-[3-(4-fluoro-benzenesulfonyl)-4-hydroxy-phenoxy]-3,5-dimethyl-phenyl}-oxamic acid; and
N-{3-chloro-4-[3-(4-fluoro-benzenesulfonyl)-4-hydroxy-phenoxy]-5-methyl-phenyl}-oxamic acid.

23. A topical pharmaceutical composition for increasing the rate of nail growth which comprises an effective amount of a compound of the formula 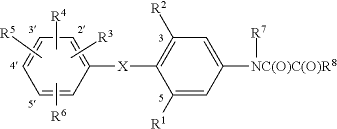
a prodrug thereof, a geometric or optical isomer thereof, or a pharmaceutically acceptable salt of said compound, said prodrug, or said isomer, wherein:
R¹ and R² are independently halogen, C₁₋₈ alkyl, —CN or C₁₋₈ perfluoroalkyl; provided that at least one of R¹ and R² is —CN;
R³ is hydrogen or C₁₋₈ alkyl;
R⁴ is halogen, C₁₋₈ perfluoroalkyl, C₁₋₈ alkyl, C₁₋₈ alkanoyl, hydroxy-(C₁₋₈ alkyl), aryl optionally substituted with Y and Z, aryl-(C₁₋₈ alkyl), carbocyclic aroyl optionally substituted with Y and Z, C₃₋₁₀ cycloalkyl optionally substituted with Y and Z, or C₃₋₁₀ cycloalkyl-(C₁₋₈ alkyl);
or R⁴ is the radical 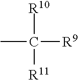
wherein: R⁹ is hydrogen, C₁₋₈ alkyl, aryl optionally substituted with Y and Z, aryl-(C₁₋₈ alkyl), C₃₋₁₀ cycloalkyl optionally substituted with Y and Z, or C₃₋₁₀ cycloalkyl-(C₁₋₈ alkyl); R¹³ is —OR¹⁴; R¹¹ is hydrogen or C₁₋₈ alkyl; or R¹⁰ and R¹¹ may be taken together with the carbon atom to which they are attached to form a carbonyl group;
R⁵ is hydroxy, esterified hydroxy or etherified hydroxy;
R⁶ is hydrogen, halogen, C₁₋₈ alkyl or C₁₋₈ perfluoroalkyl;
R⁷ is hydrogen, C₁₋₈ alkyl or C₁₋₈ perfluoroalkyl;
R⁸ is —OR¹² or —NR¹²R¹³;
R¹² and R¹³ are each independently hydrogen or C₁₋₈ alkyl;
R¹⁴ is hydrogen, C₁₋₈ alkyl or C₁₋₈ acyl;
X is O, S(O)ₐ, C═O or NR¹⁵;
a is 0, 1 or 2;
R¹⁵ is hydrogen or C₁₋₈ alkyl;
Y and Z for each occurrence are independently (a) hydrogen, (b) halogen, (c) trifluoromethyl, (d) —OCF₃, (e) —CN, (f) C₁₋₆ alkyl optionally substituted with one or more substituents independently selected from the group consisting of halogen, —OCF₃, —CF₃ and phenyl, (g) C₁₋₆ alkoxy, (h) aryl optionally substituted with one or more substituents independently selected from the group consisting of halogen, —OCF₃, —CF₃, C₁₋₄ alkyl and C₁₋₄ alkoxy, (i) —C(O)₂R¹⁶, (j) —C(O)NR¹⁶R¹⁷, (k) —C(O)R¹⁶, (l) —NR¹⁶C(O)NR¹⁶R¹⁷ or (m) —NR¹⁶C(O)R¹⁷; or Y and Z for any occurrence may be taken together to form (a) a carbocycle of the formula —(CH₂)_{b}, or (b) a heterocycle selected from the group consisting of —O(CH₂)_{c}O—, —(CH₂)_{d}NH— and —CH═CHNH—;
b is 3, 4, 5, 6 or 7;
c and d are each independently 2, 3, 4, 5 or 6;
R¹⁶ and R¹⁷ for each occurrence are independently hydrogen, C₁₋₆ alkyl, C₂₋₆ alkenyl, —(C₁₋₆ alkyl)-C₁₋₆ alkoxy, aryl optionally substituted with X and Y, het optionally substituted with X and Y, —(C₁₋₄ alkyl)-aryl optionally substituted with X and Y, —(C₁₋₄ alkyl)-heterocycle optionally substituted with X and Y, —(C₁₋₄ alkyl)-hydroxy, —(C₁₋₄ alkyl)-halo, —(C₁₋₄ alkyl)-poly-halo, —(C₁₋₄ alkyl)-CONR¹⁸R¹⁸ or C₃₋₁₀ cycloalkyl;
het for each occurrence is a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S, and including any bicyclic group in which said heterocyclic ring is fused to a benzene ring or a heterocyclic ring selected from the group consisting of 4-, 5-, 6-, 7- and 8-membered partially and fully saturated, and unsaturated, heterocyclic rings containing from one to four heteroatoms independently selected from the group consisting of N, O and S; and
R¹⁸ and R¹⁹ for each occurrence are independently hydrogen, C₁₋₆ alkyl, C₃₋₁₀ cycloalkyl or aryl optionally substituted with Y and Z;
and a pharmaceutically acceptable carrier.

24. A topical pharmaceutical composition for increasing the rate of nail growth which comprises an effective amount of a compound of the formula 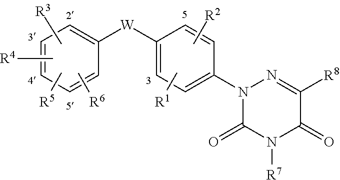
an isomer thereof, a prodrug of said compound or isomer, or a pharmaceutically acceptable salt of said compound, isomer or prodrug;
wherein W is (a) —O—, (b) —S(O)ₘ—, (c) —NR³⁰—, (d) —C(O)—, (e) —HC═CH—, (f) —CH₂—, (g) —CHF—, (h) —CF₂— or (i) —CH(OH) —;
R¹ and R² are independently (a) hydrogen, (b) halogen, (c) —(C₁-C₆)alkyl, (d) —CN, (e) —OR¹² or (f) -trifluoromethyl;
R³ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁰, (k) —C(O)R¹⁶, (l) —NR²¹C(O) —NR²¹R²², (m) —NR¹⁹—C(O)R²⁰ or (n) —NR¹⁷R¹⁸;
R⁴ is (a) —C(R¹⁴)(R¹⁵)(R¹⁶), (b) —(C₀-C₃)alkyl-NR¹⁷R¹⁸, (c) —C(O)NR¹⁹R²⁰, (d) —NR¹⁹—C(O) —R²⁰, (e) —(C₀-C₃)alkyl-NR²¹—C(O) —NR²¹R²², (f) —S(O)ₘ—R²², (g) —S(O)₂—NR²¹R²², (h) —NR²¹—S(O)₂—R²², (i) -aryl, (j) -het, (k) —OR³³ or (l) halogen; provided that in substituents (f) and (h), R²² is other than —OR³⁴; and provided that when substituent (b) is —(C₀)alkyl-NR¹⁷R¹⁸, R¹⁸ is other than —C(O) —R²⁸ or —S(O)₂—R²⁹;
or R³ and R⁴ may be taken together to form a carbocyclic ring of Formula —(CH₂)_{b}— or a heterocyclic ring selected from the group consisting of -Q-(CH₂) — and —(CH₂)ⱼ-Q-(CH₂)ₖ— wherein Q is O, S or NR²⁵; wherein said carbocyclic ring is optionally substituted with one or more substituents independently selected from Group V; and wherein said heterocyclic ring is optionally substituted with one or more substituents independently selected from Group Z;
R⁵ is —OR²³;
or R⁴ and R⁵ may be taken together to form a heterocyclic ring selected from the group consisting of —CR³¹═CR³²—NH—, —N═CR³¹—NH—, —CR³¹═CR³²—O— and —CR³¹═CR³²—S—;
R⁶ is (a) hydrogen, (b) halogen, (c) —(C₁-C₆)alkyl optionally substituted with one to three substituents independently selected from the group consisting of halogen, —OCF₃ and —CF₃, (d) —CN, (e) —OR¹², (f) -trifluoromethyl, (g) —NO₂, (h) —SO₂—R¹³, (i) —C(O)₂R⁹, (j) —C(O)NR¹⁹R²⁰, (k) —C(O)R¹⁶, (l) —NR²¹C(O)NR²¹R²², (m) —NR¹⁹—C(O)R²⁰ or (n) —NR¹⁷R¹⁸;
R⁷ is (a) hydrogen, (b) —(C₁-C₄)alkyl wherein each carbon atom is optionally substituted with 1 to 3 halo atoms or (c) —(CH₂)ₙCOOR⁹;
R⁸ is (a) hydrogen, (b) —(C₁-C₆)alkyl, (c) —C(O) —OR⁹, (d) —C(O)NR¹⁰R¹¹ or (e) —CN; provided that in substituent (c), R⁹ is other than methyl or ethyl; and provided that in substitutent (d), R¹³ and R¹¹ are not both hydrogen;
R⁹ is (a) —(C₁-C₁₂)alkyl optionally substituted with one or more substitutents independently selected from Group V, (b) —(C₂-C₁₂)alkenyl optionally substituted with phenyl, (c) —(C₂-C₁₂)dialkenyl, (d) —(C₃-C₁₀)cycloalkyl, (e) -aryl or (f) -het;
R¹⁰ and R¹¹ are independently (a) hydrogen, (b) —(C₁-C₁₂)alkyl optionally substituted with one or more substituents independently selected from Group V, (c) —(C₃-C₁₀)cycloalkyl optionally substituted with one or more substituents independently selected from Group V, (d) —(C₂-C₁₂)alkenyl or (e) -het;
or R¹⁰ and R¹¹ for any occurrence may be taken together with the nitrogen atom to which are they attached to form het;
R¹² is (a) hydrogen or (b) —(C₁-C₆)alkyl wherein each carbon atom is optionally substituted with 1 to 3 fluoro atoms;
R¹³ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) —NR¹⁷R¹⁸, (e) -aryl aryl or (f) -het;
R¹⁴ is (a) hydrogen, (b) —(C₁-C₆)alkyl or (c) —O—R³⁴;
R¹⁵ is (a) hydrogen or (b) —(C₁-C₆)alkyl;
or R¹⁴ and R¹⁵ are taken together with the carbon atom to which they are attached to form a carbonyl group;
R¹⁶ is (a) hydrogen, (b) —(C₁-C₆)alkyl wherein each carbon atom is optionally substituted with 1 to 3 fluoro atoms, (c) —(C₀-C₆)alkyl-(C₃-C₁₀)cycloalkyl, (d) —(C₀-C₆)alkyl-aryl or (e) —(C₀-C₆)alkyl-het;
R¹⁷ is (a) hydrogen, (b) —(C₁-C₁₂)alkyl optionally substituted with one or more substituents independently selected from Group V, (c) -aryl, (d) -het, (e) —OR³⁴ or (f) —(C₃-C₁₀)cycloalkyl;
R¹⁸ is (a) hydrogen, (b) —(C₁-C₁₂)alkyl optionally substituted with one or more substituents independently selected from Group V, (c) -aryl, (d) -het, (e) —C(O) —R²⁸, (f) —S(O)₂—R²⁹, (g) —OR³⁴ or (h) —(C₃-C₁₀)cycloalkyl;
or R¹⁷ and R¹⁸ for any occurrence are taken together with the nitrogen atom to which they are attached to form het;
R¹⁹ and R²⁰ for each occurrence are independently (a) hydrogen, (b) —(C₁-C₁₂)alkyl optionally substituted with one or more substituents independently selected from Group V, (c) —(C₀-C₆)alkyl-aryl, (d) —(C₀-C₆)alkyl-het, (e) —C(O) —NR²⁶R²⁷, (f) —C(O) —R²⁸, (g) —S(O)₂—R²⁹, (h) —OR³⁴ or (i) —(C₃-C₁₀)cycloalkyl;
or R¹⁹ and R²⁹ for any occurrence are taken together with the nitrogen atom to which they are attached to form het;
R²¹ and R²² for each occurrence are independently (a) hydrogen, (b) —(C₁-C₁₂)alkyl optionally substituted with one to three substituents independently selected from Group V, (c) -aryl, (d) -het, (e) —(C₃-C₁₀)cycloalkyl or (f) —OR³⁴;
or R²¹ and R²² are taken together with the nitrogen atom to which they are attached to form het;
R²³ is (a) hydrogen, (b) —(C₁-C₄)alkyl optionally substituted with one or more substituents independently selected from Group V or (c) —C(O) —R²⁴;
R²⁴ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R²⁵ for each occurrence is independently (a) hydrogen, (b) —(C₁-C₆)alkyl, (c) —COR²⁹ or (d) —SO₂R²⁹;
R²⁶ and R²⁷ for each occurrence are independently (a) hydrogen, (b) —(C₁-C₆)alkyl, (c) —(C₃-C₁₀)cycloalkyl, (d) —(C₀-C₆)alkyl-aryl, or (e) —(C₀-C₆)alkyl-het,
R²⁸ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R²⁹ is (a) —(C₁-C₁₂)alkyl optionally substituted with one or more substituents independently selected from Group V, (b) —(C₂-C₁₂)alkenyl, (c) —(C₃-C₁₀)cycloalkyl, (d) -aryl or (e) -het;
R³⁰ is (a) hydrogen, (b) —(C₁-C₁₂)alkyl optionally substituted with one or more substituents independently selected from Group V, (c) —(C₁-C₁₂)alkenyl, (d) —(C₃-C₁₀)cycloalkyl, (e) —C(O) —R³¹ or (f) —S(O)ₘ—R³²;
R³¹ is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl, (f) -het or (g) —OR³⁴;
R³² is (a) hydrogen, (b) —(C₁-C₁₂)alkyl optionally substituted with one or more substituents independently selected from Group V, (c) —(C₂-C₁₂)alkenyl, (d) —(C₃-C₁₀)cycloalkyl, (e) -aryl or (f) -het;
R³³ is (a) —(C₀-C₆)alkyl-aryl, (b) —(C₀-C₆)alkyl-het, (c) —(C₇-C₁₂)alkyl optionally substituted with one or more substituents independently selected from Group V, (d) —(C₁-C₆)alkyl wherein at least one carbon atom is substituted with 1 to 3 fluoro atoms, (e) —(C₂-C₁₂)alkenyl or (f) —(C₃-C₁₀)cycloalkyl;
R³⁴ is (a) -aryl, (b) -het, (c) —(C₁-C₁₂)alkyl optionally substituted with one or more substituents independently selected from Group V, (d) —(C₂-C₁₂)alkenyl or (e)-(C₃-C₁₀)cycloalkyl;
—(C₃-C₁₀)cycloalkyl for each occurrence is a fully or partially saturated mono-, bi- or tricyclic ring containing three to ten carbon atoms; wherein in the bicyclic ring, a monocyclic cycloalkyl ring is spiro used to another cycloalkyl ring or is fused via two carbon atoms to a benzene ring or another cycloalkyl ring; and wherein in the tricyclic ring, a bicyclic ring is spiro used to a cycloalkyl ring or is fused via two atoms to a benzene ring or another cycloalkyl ring;
said —(C₃-C₁₀)cycloalkyl optionally contains one to three bridging atoms independently selected from carbon, oxygen, sulfur and nitrogen; said bridging atoms are attached to two carbon atoms in the ring; and said bridging atoms are optionally substituted with one to three groups independently selected from —(C₁-C₆)alkyl and hydroxy;
said cycloalkyl ring is optionally substituted on one ring if the moiety is monocyclic, on one or both rings if the moiety is bicyclic, or on one, two or three rings if the moiety is tricyclic, with one or more substitutents independently selected from Group V;
Group V is (a) —(C₁-C₆)alkyl optionally substituted with one or two hydroxy, (b) —(C₂-C₅)alkynyl, (c) -halogen, (d) —NR³⁵R³⁶, (e) —NO₂, (f) —OCF₃, (g) —OR³⁷, (h) —SR³⁷, (i) -oxo, (j) -trifluoromethyl, (k) —CN, (l) —C(O)NR³⁵—OH, (m) —COOR³⁵, (n) —O—C(O) —(C₁-C₆)alkyl, (O) —(C₃-C₁₀)cycloalkyl optionally substituted with CN, (p) —(C₀-C₆)alkyl-aryl, (q) —(C₀-C₆)alkyl-het, (r) —C(O)—(C₁-C₆)alkyl or (s) —C(O)-aryl;
R³⁵ and R³⁶ for each occurrence are independently (a) hydrogen, (b) —(C₁-C₆)alkyl or (c) —(C₀-C₆)alkyl-aryl;
R³⁷ is (a) hydrogen, (b) —(C₁-C₆)alkyl optionally substituted with one or more halo, hydroxy or methoxy, (c) —(C₀-C₆)alkyl-aryl or (d) —(C₀-C₆)alkyl-het;
aryl is (a) phenyl optionally substituted with one or more substituents independently selected from Group Z; (b) naphthyl optionally substituted with one or more substituents independently selected from Group Z or (c) biphenyl optionally substituted with one or more substituents independently selected from Group Z;
het for each occurrence is a 4-, 5-, 6-, 7- and 8-membered fully saturated, partially saturated or fully unsaturated mono-, bi- or tricyclic heterocyclic ring containing from one to four heteroatoms independently selected from the group consisting of oxygen, sulfur and nitrogen; wherein in the bicyclic ring, a monocyclic heterocyclic ring is spiro fused to a —(C₃-C₈)cycloalkyl ring or to another heterocyclic ring which is fully or partially saturated; or is fused via two atoms to a benzene ring, a —(C₃-C₈)cycloalkyl ring or another heterocyclic ring; and wherein in the tricyclic ring, a bicyclic ring is spiro fused to a —(C₃-C₈)cycloalkyl ring or to another heterocyclic ring which is fully or partially saturated; or is fused via two atoms to a benzene ring, a (C₃-C₆)cycloalkyl ring, or another heterocyclic ring;
said het optionally contains one to three bridging atoms independently selected from oxygen, sulfur and nitrogen; said bridging atoms are attached to two other atoms in the ring; and said bridging atoms are optionally substituted with one to three groups independently selected from —(C₁-C₆)alkyl and hydroxy;
said het optionally has one or two oxo groups substituted on carbon or one or two oxo groups substituted on sulfur;
said het is optionally substituted on carbon or nitrogen, on one ring if the moiety is monocyclic, on one or both rings if the moiety is bicyclic, or on one, two or three rings if the moiety is tricyclic, with one or more substituents independently selected from Group Z;
Group Z for each occurrence is independently (a) hydrogen, (b) halogen, (c) trifluoromethyl, (d) hydroxy, (e) —OCF₃, (f) —CN, (g) —NO₂, (h) —(C₁-C₆)alkyl optionally substituted with one or more substituents independently selected from the group consisting of hydroxy, halogen, —OCF₃ and —CF₃, (i) —(C₂-C₆)alkenyl optionally substituted with phenyl, (j) —(C₂-C₅)alkynyl, (k) —(C₁-C₆)alkoxy, (l) —(C₀-C₆)alkyl-phenyl optionally substituted with one or more substituents independently selected from the group consisting of halogen, —OCF₃, —CF₃, —(C₁-C₄)alkyl, —(C₁-C₄)alkoxy and —C(O)CH₃, (m) —(C₀-C₆)alkyl-naphthyl optionally substituted with one or more substituents independently selected from the group consisting of halogen, —OCF₃, —CF₃, —(C₁-C₄)alkyl, —(C₁-C₄)alkoxy and —C(O)CH₃, (n) —C(O)₂R³⁵, (O) —(C₀-C₆)alkyl-C(O)NR³⁵R³⁶, (p) —(C₀-C₆)alkyl —C(O)R³⁸, (q) —NR³⁵R³⁶, (r) —NR³⁵—C(O)NR³⁵R³⁶, (s) —NR³⁵—C(O)R³⁶, (t) —OR³⁷, (u) —SR³⁷, (v) —(C₃-C₁₀)cycloalkyl, (w)-(C₀-C₆)alkyl-pyridinyl optionally substituted with one or more —(C₁-C₆)alkyl which is optionally substituted with one or more substituents independently selected from the group consisting of hydroxy and halo, (x) —(C₀-C₆)alkyl-piperidinyl optionally substituted with one or more —(C₁-C₆)alkyl which is optionally substituted with one or more substituents independently selected from hydroxy and halo, (y) —SO₂—R³⁷, (z) —SO₂—NR³⁵R³⁶ or (a1) —S-phenyl-CH₂OH;
R³⁸ is (a) —(C₁-C₆)alkyl, (b) —(C₀-C₆)alkyl-phenyl, (c) —(C₀-C₆)alkyl-phenanthrenyl optionally substituted with one to three CF₃, (d) —(C₀-C₆)alkyl-Pyrrolidinyl or (e) —(C₀-C₆)alkyl-morpholinyl;
or any two Z Groups for any occurrence in the same variable may be taken together to form (a) a carbocyclic ring of the formula —(CH₂)ₑ— or (b) a heterocyclic ring selected from the group consisting of —O(CH₂)_{f}O—, —(CH₂)_{g}NH— and —CH═CHNH—;
m is 0, 1 or 2;
n is 0, 1, 2 or 3;
b is 3, 4, 5, 6 or 7;
c, f, g, j and k are each independently 2, 3, 4, 5 or 6; and
e is 3, 4, 5, 6 or 7;
provided that in a compound of the above formula: 1) the substituent —C(R¹⁴)(R¹⁵)(R¹⁸) in R⁴ is other than (C₁-C₄)alkyl; and 2) R⁴ is halo only when R⁸ is —C(O) —OR⁹ or —C(O)NR¹⁰R¹¹;
and a pharmaceutically acceptable carrier.

25. A composition of claim 24 wherein the compound is selected from the group consisting of:
8-[[5-[2,6-dichloro-4-(4,5-dihydro-3,5-dioxo-1,2,4-triazine-2(3H)-yl)phenoxy]-2-hydroxyphenyl]sulfonyl]-spiro[8-azabicyclo[3.2.1]octane-3,2′-(3′H)-dihydro-furan];
2-{3,5-dichloro-4-[3-(3,3-dimethyl-piperidine-1-sulfonyl)-4-hydroxy-phenoxy]-phenyl}-2H-[1,2,4]triazine-3,5-dione;
2-{3,5-dichloro-4-[4-hydroxy-3-(3-methyl-3-phenyl-piperidine-1-sulfonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzenesulfonamide;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,3-dimethyl-piperidine-1-carbonyl)-4-hydroxy-phenoxy]-phenyl}-2H-[1,2,4]triazine-3,5-dione;
N-bicyclo[2.2.1]hept-2-yl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[4-hydroxy-3-(3-methyl-3-phenyl-piperidine-1-carbonyl)-phenoxy]-phenyl}-2H-[1,2,4]triazine-3,5-dione;
5-[2,6-dichloro-4-(3,5-dioxo-4,5-dihydro-3H-[1,2,4]triazin-2-yl)-phenoxy]-N-(6,6-dimethyl-bicyclo[3.1.1]hept-2-yl)-2-hydroxy-benzamide;
2-{3,5-dichloro-4-[3-(3,5-dimethyl-piperidine-1-carbonyl)-4-hydroxy-phenoxy]-phenyl}-2H-[1,2,4]triazine-3,5-dione;
2-{3,5-dichloro-4-[4-hydroxy-3-(piperidine-1-carbonyl)-phenoxy]-phenyl}-2H-[1,2,4]triazine-3,5-dione;
N-cyclohexyl-5-[2,6-dichloro-4-(3,5-dioxo-4,5-dihydro-3H-[1,2,4]triazin-2-yl)-phenoxy]-2-hydroxy-benzamide;
2-{3,5-dichloro-4-[3-(3,4-dihydro-1H-isoquinoline-2-carbonyl)-4-hydroxy-phenoxy]-phenyl}-2H-[1,2,4]triazine-3,5-dione;
2-{4-[3-(4-fluoro-benzyl)-4-hydroxy-phenoxy]-3,5-dimethyl-phenyl}-2H-[1,2,4]triazine-3,5-dione; and
2-{3,5-dichloro-4-[3-(4-fluoro-benzoyl)-4-hydroxy-phenoxy]-phenyl}-2H-[1,2,4]triazine-3,5-dione.

26. A topical pharmaceutical composition for increasing the rate of nail growth which comprises an effective amount of a compound of the formula 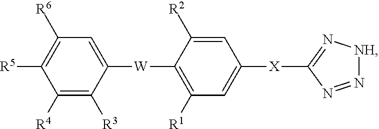
or a stereoisomer, a pharmaceutically acceptable salt or prodrug thereof, or a pharmaceutically acceptable salt of the prodrug, wherein:
W is O, S, SO, SO₂, CH₂, CF₂, CHF, C(═O), CH(OH), NR^{a}, or 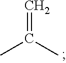
X is O, CH₂, CH₂CH₂, S, SO, SO₂, CH₂NR^{a}, NR^{a}, or a bond;
each R^{a} is independently hydrogen, C₁-C₆alkyl, or C₁-C₆alkyl substituted with one substituent selected from C₃-C₆cycloalkyl or methoxy;
R¹, R², R³ and R⁶ are independently hydrogen, halogen, C₁-C₈alkyl, —CF₃, —OCF₃, —OC₁-C₈alkyl, or —CN;
R⁴ is hydrogen, C₁-C₁₂alkyl, [C₁-C₁₂alkyl that is substituted with from one to three substituents independently selected from Group V], C₂-C₁₂ alkenyl, C₂-C₁₂ alkynyl, halogen, —CN, —SR^{b}, —S(═O)R^{c}, —S(═O)₂R^{c}, aryl, heteroaryl, C₃-C₁₀ cycloalkyl, heterocycloalkyl, —S(═O)₂NR^{c}R^{d}, —C(═O)NR^{c}R^{d}, —C(═O)OR^{c}, —NR^{a}C(═O)R^{d}, —NR^{a}C(═O)NR^{c}R^{d}, —NR^{a}S(═O)₂R^{d}, —NR^{a}R^{d}, —C(═O)R^{c},
or R³ and R⁴ may be taken together with the carbon atoms to which they are attached to form an unsubstituted or substituted carbocyclic ring of formula —(CH₂) or an unsubstituted or substituted heterocyclic ring selected from the group consisting of -Q-(CH₂)ⱼ— and —(CH₂)ₖ—O—(CH₂)ₗ— wherein Q is O, S or NR^{a}; i is 3, 4, 5, 6 or 7; j is 2, 3, 4, 5, or 6; k and l are each independently 1, 2, 3, 4, or 5, and any substituents up to four are selected from C₁-C₄alkyl, —OR^{b}, oxo, —CN, phenyl, or —NR^{a}R^{g};
R^{b} is hydrogen, C₁-C₁₂alkyl, [C₁-C₁₂alkyl substituted with one to three substituents independently selected from Group V], aryl, heteroaryl, C₃-C₁₀ cycloalkyl, heterocycloalkyl, —C(═O)NR^{c}R^{d}, or —C(═O)R^{f};
R^{c} and R^{d} are each independently selected from hydrogen, C₁-C₁₂alkyl, [C₁-C₁₂alkyl substituted with one to three substituents independently selected from Group VI], C₂-C₁₂alkenyl, C₂-C₁₂alkynyl, aryl, heteroaryl, C₃-C₁₀ cycloalkyl, heterocycloalkyl,
or R^{c} and R^{d} may together along with the atom(s) to which they are attached form a 3-10 membered unsubstituted or substituted heterocyclic ring, which may contain a second heterogroup selected from O, NR^{e}, or S, wherein any substitutents up to four are selected from C₁-C₄alkyl, —OR^{b}, oxo, —CN, phenyl, or —NR^{a}R^{g};
R⁵ is —OH, —OC₁-C₆alkyl, —OC(═O)R^{f}, —F, —C(═O)OR^{c},
or R⁴ and R⁵ may together with the atom(s) to which they are attached form a heterocyclic ring selected from the group consisting of —CR^{c}═CR^{a}—NH—, —N═CR^{a}—NH—, —CR^{c}═CR^{a}—O—, —CR^{c}═CR^{a}—S—, —CR^{c}═N—NH—, or —CR^{a}═CR^{a}—CR^{a}═N—;
Group V is halogen, —CF₃, —OCF₃, hydroxy, oxo, C₁-C₆alkoxy, —CN, aryl, heteroaryl, C₃-C₁₀cycloalkyl, heterocycloalkyl, —SR^{f}, —S(═O)R^{f}, —S(═O)₂R^{f}, [—S(═O)₂NR^{a}R^{f}, wherein R^{a} and R^{f} may together along with the atom(s) to which they are attached form a 3-8 membered heterocyclic ring, which may contain a second heterogroup selected from O, NR^{e} or S], —NR^{a}R^{g}, or [—C(═O)NR^{a}R^{f},
wherein R^{a} and R^{f} may together along with the atom(s) to which they are attached form a 3-8 membered heterocyclic ring, which may contain a second heterogroup selected from O, NR^{e} or S];
Group VI is halogen, hydroxy, oxo, C₁-C₆alkoxy, aryl, heteroaryl, C₃-C₈cycloalkyl, heterocycloalkyl, —CN, or —OCF₃;
R^{e} is hydrogen, —CN, C₁-C₁₀alkyl, [C₁-C₁₀alkyl substituted with one to three substitutents independently selected from Group V], C₂-C₁₀alkenyl, C₂-C₁₈alkoxy, C₃-C₁₀cycloalkyl, aryl, heteroaryl, —C(═O)R^{f}, —C(═O)OR^{f}, —C(═O)NR^{a}R^{f}, —S(═O)₂NR^{a}R^{f}, or
—S(═O)₂R^{f};
R^{f} is hydrogen, C₁-C₁₀alkyl, [C₁-C₁₀alkyl substituted with from one to three substituents selected from Group VI], C₂-C₁₀alkenyl, C₂-C₁₀alkoxy, C₃-C₁₀cycloalkyl, heterocycloalkyl, aryl, or heteroaryl; and
R⁹ is hydrogen, C₁-C₆alkyl, C₃-C₈cycloalkyl, C₂-C₆ alkenyl, aryl, —C(═O)R^{f}, —C(═O)OR^{f}, —C(═O)NR^{a}R^{f}, or —S(═O)₂R^{f}, provided that R¹ and R² are not both hydrogen, further provided that when X is CH₂, W is NR^{a}, R³ is hydrogen and R⁵ is —OH, then R⁶ and R⁴ are not both —C(CH₃)₃, further provided that when X is CH₂ or CH₂CH₂, W is O, and R³ and R⁶ are hydrogen, then R⁴ is not halogen, —CF₃, C₁-C₆alkyl or C₃-C₇cycloalkyl, and further provided that when R³ and R⁴ are hydrogen and W is O then R⁶ is not halogen, —CF₃, C₁-C₆alkyl or C₃-C₇cycloalkyl;
and a pharmaceutically acceptable carrier.

27. A topical pharmaceutical composition for increasing the rate of nail growth which comprises an effective amount of a compound of the formula 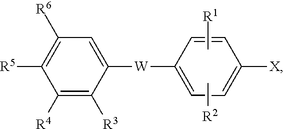
a stereoisomer or prodrug thereof, or a pharmaceutically acceptable salt of said compound, stereoisomer, or prodrug, wherein:
W is oxygen, sulfur, —SO—, —S(O)₂, —CH₂—, —CF₂—, —CHF—, —C(O) —, —CH(OH) —, —NR^{a}, or —C(═CH₂) —;
R¹, R², R³, and R⁶ are each independently hydrogen, halogen, —(C₁-C₈)alkyl, —CF₃, —OCF₃, —O(C₁-C₈)alkyl, or —CN;
R⁴ is hydrogen, —(C₁-C₁₂)alkyl substituted with zero to three substituents independently selected from Group V, —(C₂-C₁₂)alkenyl, —(C₂-C₁₂)alkynyl, halogen, —CN, —OR^{b}, —S(O)R^{c}, —S(O)₂R^{c}, aryl, heteroaryl, —(C₃-C₁₀)cycloalkyl, heterocycloalkyl, —S(O)₂NR^{c}R^{d}, —C(O)NR^{c}R^{d}, —C(O)OR^{c}, —NR^{a}C(O)R^{d}, —NR^{a}C(O)NR^{c}R^{d}, —NR^{a}S(O)₂R^{d}, or —C(O)R^{d}; or
R³ and R⁴ are taken together along with the carbon atoms to which they are attached to form a carbocyclic ring of formula —(CH₂)ᵢ— or a heterocyclic ring of formula —(CH₂)ₖ-Q-(CH₂)ᵢ— wherein Q is oxygen, sulfur, or —NR^{e}—; i is 3, 4, 5, or 6; k is 0, 1, 2, 3, 4, or 5; and l is 0, 1, 2, 3, 4, or 5; and wherein said carbocyclic ring and said heterocyclic ring are each substituted with zero to four substituents independently selected from —(C₁-C₄)alkyl, —OR^{b}, oxo, —CN, phenyl, or —NR^{a}R^{g};
R⁵ is hydroxy, —O(C₁-C₆)alkyl, —OC(O)R^{f}, fluorine, or —C(O)OR^{c}; or
R⁴ and R⁵ are taken together along with the carbon atoms to which they are attached to form a heterocyclic ring selected from the group consisting of —CR^{c}═-CR^{a}—NH—, —N═CR^{a}—NH, —CR^{a}═CR^{a}—S—, —CR^{a}═N—NH—, and —CR^{a}═CR^{a}—CR^{a}═N—;
R^{a} for each occurrence is independently hydrogen, or —(C₁-C₆)alkyl substituted with zero or one —(C₃-C₆)cycloalkyl or methoxy;
R^{b} for each occurrence is independently hydrogen, —(C₁-C₁₂)alkyl substituted with zero to three substituents independently selected from Group V, aryl, heteroaryl, —(C₃-C₁₀)cycloalkyl, heterocycloalkyl, —C(O)NR^{c}R^{d}, or —C(O)R^{f};
R^{c} and R^{d} for each occurrence are each independently hydrogen, —(C₁-C₁₂)alkyl substituted with zero to three substituents independently selected from Group VI, —(C₂-C₁₂)alkenyl, —(C₂-C₁₂)alkynyl, aryl, heteroaryl, —(C₃-C₁₀)cycloalkyl, or heterocycloalkyl;
provided that when R⁴ is the moiety —SR^{c}, —S(O)R^{c}, or —S(O)₂R^{c}, R^{c} is other than hydrogen; or
R^{c} and R^{d} are taken together along with the atom(s) to which they are attached to form a 3-10 membered heterocylic ring which may optionally contain a second heterogroup selected from oxygen, —NR^{e}—, or sulfur; and wherein said heterocyclic ring is substituted with zero to four substituents independently selected from —(C₁-C₄)alkyl, —OR^{b}, oxo, —CN, phenyl, or —NR^{a}R^{g};
R^{e} for each occurrence is hydrogen, —CN, —(C₁-C₁₀)alkyl substituted with zero to three substituents independently selected from Group V, —(C₂-C₁₀)alkenyl, —(C₂-C₁₀)alkoxy, —(C₃-C₁₀)cycloalkyl, aryl, heteroaryl, —C(O)R^{f}, —C(O)OR^{f}, —C(O)NR^{a}R^{f}, or —S(O)₂R^{f};
R^{f} for each occurrence is independently —(C₁-C₁₀)alkyl substituted with zero to three substituents independently selected from Group VI, —(C₂-C₁₂)alkenyl, —(C₂-C₁₀)alkynyl, —(C₃-C₁₀)cycloalkyl, aryl, heteroaryl, or heterocycloalkyl;
R^{g} for each occurrence is independently hydrogen, —(C₁-C₆)alkyl, —(C₂-C₆)alkenyl, aryl, —C(O)R^{f}, —C(O)OR^{f}, —C(O)NR^{a}R^{f}, —S(O)₂R^{f}, or —(C₃-C₈)cycloalkyl;
Group V is halogen, —CF₃, —OCF₃, —OH, oxo, —(C₁-C₆)alkoxy, —CN, aryl, heteroaryl, —(C₃-C₁₀)cycloalkyl, heterocycloalkyl, —SR^{f}, —S(O)R^{f}, —S(O)₂R^{f}, —S(O)₂NR^{a}R^{f}, —NR^{a}R^{g}, or —C(O)NR^{a}R^{f};
Group VI is halogen, hydroxy, oxo, —(C₁-C₆)alkoxy, aryl, heteroaryl, —(C₃-C₈)cycloalkyl, heterocycloalkyl, —CN, or —OCF₃;
provided that when R⁴ is —(C₁-C₁₂)alkyl substituted with zero to three substituents independently selected from Group V, wherein said Group V substituent is oxo, said oxo group is substituted on a carbon atom other than the C₁ carbon atom in —(C₁-C₁₂)alkyl;
aryl for each occurrence is independently phenyl or naphthyl substituted with zero to four substituents independently selected from halogen, —(C₁-C₆)alkyl, —CN, —SR^{f}, —S(O)R^{f}, —S(O)₂R^{f}, —(C₃-C₆)cycloalkyl, —S(O)₂NR^{a}R^{f}, —NR^{a}R^{g}, —C(O)NR^{a}R^{f}, —OR^{b}, -perfluoro-(C₁-C₄)alkyl, or —COOR^{f};
provided that when said substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, said substituents R^{b}, R^{f}, and R^{g}, are other than aryl or heteroaryl;
heteroaryl for each occurrence is independently a 5-, 6-, 7-, 8-, or 9-membered monocyclic or bicyclic ring having from one to three heteroatoms selected from O, N, or S;
wherein in said bicyclic ring, a monocyclic heteroaryl ring is fused to a benzene ring or to another heteroaryl ring, and having zero to three substituents independently selected from halogen, —(C₁-C₄)alkyl, —CF₃, —OR^{b}, —NR^{a}R^{g}, or —COOR^{f};
provided that when said substituent(s) on heteroaryl are —NR^{a}R^{g}, —OR^{b}, or —COOR^{f}, said substituents R^{b}, R^{f}, and R^{g}, are other than aryl or heteroaryl;
heterocycloalkyl for each occurrence is independently a 5-, 6-, 7-, 8-, or 9-membered monocyclic or bicyclic cycloalkyl ring having from one to three heteroatoms selected from oxygen, —NR^{a}, or sulfur, and having zero to four substituents independently selected from —(C₁-C₄)alkyl, —OR^{b}, oxo, —CN, phenyl, or —NR^{a}R^{g}; and
X is 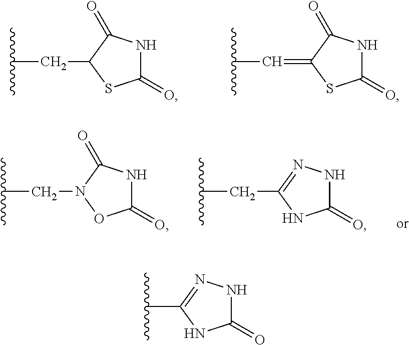
and a pharmaceutically acceptable carrier.

28. A topical pharmaceutical composition for increasing the rate of nail growth which comprises an effective amount of a compound of the formula 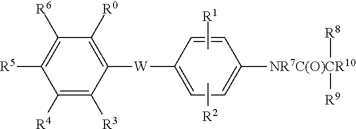
an isomer thereof, a prodrug of said compound or isomer, or a pharmaceutically acceptable salt of said compound, isomer or prodrug;
wherein W is (a) —O—, (b) —S—, (c) —SO—, (d) —SO₂—, (e) —CH₂—, (f) —CF₂—, (g) —CHF—, (h) —C(O) —, (i) —CH(OH) —, (j) —NR^{a} or (k) 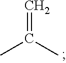
R⁰ is (a) hydrogen, (b) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (c) —C(O)R^{h}, (d) —S(O)₂R^{h} or (e) halogen;
R¹, R², R³ and R⁶ are each independently (a) hydrogen, (b) halogen, (c) —(C₁-C₈)alkyl, (d) —CF₃, (e) —OCF₃, (f) —O(C₁-C₈)alkyl, or (g) —CN;
R⁴ is (a) hydrogen, (b) —(C₁-C₁₂)alkyl substituted with zero to three substituents independently selected from Group V, (c) —(C₂-C₁₂)alkenyl, (d) —(C₂-C₁₂)alkynyl, (e) halogen, (f) —CN, (g) —OR^{b}, (h) —SR^{c}, (i) —S(O)R^{c}, (j) —S(O)₂R^{c}, (k) aryl, (l) heteroaryl, (m) —(C₃-C₁₀)cycloalkyl, (n) heterocycloalkyl, (O) —S(O)₂NR^{c}R^{d}, (p) —C(O)NR^{e}R^{d}, (q) —C(O)OR^{c}, (r) —NR^{a}C(O)R^{d}, (s) —NR^{a}C(O)NR^{c}R^{d}, (t) —NR^{a}S(O)₂R^{d}, (u) —NR^{a}R^{d} or (v) —C(O)R^{c};
or R³ and R⁴ are taken together along with the carbon atoms to which they are attached to form a carbocyclic ring of formula —(CH₂)ᵢ— or a heterocyclic ring of formula —(CH₂)ₖ—O—(CH₂)ᵣ wherein Q is —O—, —S— or —NR^{c}—; i is 3, 4, 5 or 6; k is 0, 1, 2, 3, 4 or 5; and l is 0, 1, 2, 3, 4 or 5; and wherein the carbocyclic ring and the heterocyclic ring are each substituted with zero to four substituents independently selected from (a) —(C₁-C₄)alkyl, (b) (c) oxo, (d) —CN, (e) phenyl or (f) —NR^{a}R^{g};
R⁵ is (a) —OH, (b) —O(C₁-C₆)alkyl, (c) —OC(O)R^{f}, (d) F, or (e) —C(O)OR^{c};
or R⁴ and R⁵ are taken together along with the carbon atoms to which they are attached to form a heterocyclic ring selected from the group consisting of —CR^{c}═CR^{a}—NH—, —N═CR^{a}—NH, —CR^{a}═CR^{a}—O—, —CR^{c}═CR^{a}—S—, —CR^{c}═N—NH— and —CR^{a}═-CR^{a}—CR^{a}═N—;
R⁷ is (a) hydrogen or (b) —(C₁-C₆)alkyl;
R⁸ and R⁹ are each independently (a) hydrogen, (b) —(C₁-C₆)alkyl, (c) aryl, or (d) halogen;
R¹⁰ is (a) —(C₀-C₁)alkyl-C(O)OH, (b) —(C₀-C₁)alkyl-C(O)OR^{f}, (c) —(C₀-C₁)alkyl-C(O)NR^{c}R^{d}, or (d) —(C₀-C₁)alkyl-OH;
R^{a} for each occurrence is independently (a) hydrogen or (b) —(C₁-C₆)alkyl substituted with zero or one —(C₃-C₆)cycloalkyl or methoxy;
R^{b} for each occurrence is independently (a) hydrogen, (b) —(C₁-C₁₂)alkyl substituted with zero to three substituents independently selected from Group V, (c) aryl, (d) heteroaryl, (e) —(C₃-C₁₀)cycloalkyl, (f) heterocycloalkyl, (g) —C(O)NR^{c}R^{d}, or (h) —C(O)R^{f};
R^{c} and R^{d} for each occurrence are each independently (a) hydrogen, (b) —(C₁-C₁₂)alkyl substituted with zero to three substituents independently selected from Group VI, (c) —(C₂-C₁₂)alkenyl, (d) —(C₂-C₁₂)alkynyl, (e) aryl, (f) heteroaryl, (g) —(C₃-C₁₀)cycloalkyl or (h) heterocycloalkyl;
provided that when R⁴ is the moiety —SR^{c}, —S(O)R^{c} or —S(O)₂R^{c}, R^{c} is other than hydrogen;
or R^{c} and R^{d} are taken together along with the atom(s) to which they are attached to form a 3-10 membered heterocyclic ring which may optionally contain a second heterogroup selected from —O—, —NR^{e}— or —S—; and wherein the heterocyclic ring is substituted with zero to four substituents independently selected from (a) —(C₁-C₄)alkyl, (b) —OR^{b}, (c) oxo, (d) —CN, (e) phenyl or (f) —NR^{a}R^{g};
R^{e} for each occurrence is (a) hydrogen, (b) —CN, (c) —(C₁-C₁₀)alkyl substituted with zero to three substitutents independently selected from Group V, (d) —(C₂-C₁₀)alkenyl, (e) —(C₂-C₁₀)alkoxy, (f) —(C₃-C₁₀)cycloalkyl, (g) aryl, (h) heteroaryl, (i) —C(O)R^{f}, (j) —C(O)OR^{f}, (k) —C(O)NR^{a}R^{f} or (l) —S(O)₂R^{f};
R^{f} for each occurrence is independently (a) —(C₁-C₁₀)alkyl substituted with zero to three substituents independently selected from the Group VI, (b) —(C₂-C₁₀)alkenyl, (c) —(C₂-C₁₀)alkynyl, (d) —(C₃-C₁₀)cycloalkyl, (e) aryl, (f) heteroaryl or (g) heterocycloalkyl;
R^{g} for each occurrence is independently (a) hydrogen, (b) —(C₁-C₆)alkyl, (c)-(C₂-C₆)alkenyl, (d) aryl, (e) —C(O)R^{f}, (f) —C(O)OR^{f}, (g) —C(O)NR^{a}R^{f}, (h) —S(O)₂R^{f} or (i) —(C₃-C₈)cycloalkyl;
R^{h} is (a) —(C₁-C₆)alkyl substituted with zero or one substituent selected from the group consisting of (1) —(C₃-C₆)cycloalkyl, (2) heterocycloalkyl and (3) phenyl substituted with zero or one substituent selected from the group consisting of (i) —(C₁-C₄)alkyl, (ii) halogen, (iii) —CF₃ and (iv) —OCF₃; (b) phenyl substituted with zero to two substituents independently selected from the group consisting of (1) —(C₁-C₄)alkyl, (2) halogen, (3) —CF₃ and (4) —OCF₃; (c) —(C₃-C₆)cycloalkyl or (d) heterocycloalkyl;
Group V is (a) halogen, (b) —CF₃, (c) —OCF₃, (d) —OH, (e) -oxo, (f) —(C₁-C₆)alkoxy, (g) —CN, (h) aryl, (i) heteroaryl, (j) —(C₃-C₁₀)cycloalkyl, (k) heterocycloalkyl, (l) —SR^{f}, (m) —S(O)R^{f}, (n) —S(O)₂R^{f}, (O) —S(O)₂NR^{a}R^{f} (p) —NR^{a}R^{g} or (q) —C(O)NR^{a}R^{f};
Group VI is (a) halogen, (b) hydroxy, (c) oxo, (d) —(C₁-C₆)alkoxy, (e) aryl, (f) heteroaryl, (g) —(C₃-C₈)cycloalkyl, (h) heterocycloalkyl, (i) —CN, or (j) —OCF₃;
provided that when the substituent R⁴ is —(C₁-C₁₂)alkyl substituted with zero to three substituents independently selected from Group V wherein the Group V substituent is oxo, the oxo group is substituted on a carbon atom other than the C₁ carbon atom in —(C₁-C₁₂)alkyl;
aryl for each occurrence is independently phenyl or naphthyl substituted with zero to four substituents independently selected from (a) halogen, (b) —(C₁-C₆)alkyl, (c) —CN, (d) (e) —S(O)R^{f}, (f) —S(O)₂R^{f}, (g) —(C₃-C₆)cycloalkyl, (h) —S(O)₂NR^{a}R^{f}, (i) —NR^{a}R^{g}, (j) —C(O)NR^{a}R^{g}, (k) (l) -perfluoro-(C₁-C₄)alkyl, or (m) —COOR^{f};
provided that when the substituent(s) on aryl are —SR^{f}, —S(O)R^{f}, —S(O)₂R^{f}, —S(O)₂NR^{a}R^{f}, —NR^{a}R^{g}, —C(O)NR^{a}R^{f}, —OR^{b}, or —COOR^{f}, the substituents R^{b}, R^{f} and R^{g} are other than aryl or heteroaryl;
heteroaryl for each occurrence is independently a 5-, 6-, 7-, 8-, 9- or 10-membered monocyclic or bicyclic ring having from 1 to 3 heteroatoms selected from O, N or S; wherein in the bicyclic ring, a monocyclic heteroaryl ring is fused to a benzene ring or to another heteroaryl ring; and having zero to three substituents independently selected from (a) halogen, (b) —(C₁-C₄)alkyl, (c) —CF₃, (d) (e) —NR^{a}R^{g}, or (f) —CO₂R^{f};
provided that when the substituent(s) on heteroaryl are —OR^{b}, —NR^{a}R^{g} or —CO₂R^{f}, the substituents R^{b}, R^{f} and R^{g} are other than aryl or heteroaryl;
heterocycloalkyl for each occurrence is independently a 4-, 5-, 6-, 7-, 8-, 9- or 10-membered monocyclic or bicyclic cycloalkyl ring having from 1 to 3 heteroatoms selected from O, NR^{e} or S; and having zero to four substituents independently selected from (a) —(C₁-C₄)alkyl, (b) —OR^{b}, (c) oxo, (d) —CN, (e) phenyl or (f) —NR^{a}R^{g};
and a pharmaceutically acceptable carrier.

29. A composition of claim 28 wherein the compound is selected from the group of:
N-{4-[3-(cyclobutyl-methyl-carbamoyl)-4-hydroxy-phenoxy]-3,5-dimethyl-phenyl}-malonamic acid;
N-{3-chloro-4-[4-hydroxy-3-(1-isopropyl-2-methyl-propylcarbamoyl)-phenoxy]-5-methyl-phenyl}-malonamic acid;
N-{3,5-dichloro-4-[3-((1S)-cyclohexyl-ethylcarbamoyl)-4-hydroxy-phenoxy]-phenyl}-malonamic acid;
N-[3,5-dichloro-4-(3-cyclopropylsulfamoyl-4-hydroxy-phenoxy)-phenyl]-malonamic acid;
N-[3,5-dichloro-4-(3-cyclobutylsulfamoyl-4-hydroxy-phenoxy)-phenyl]-malonamic acid;
N-[3-chloro-4-(3-cyclobutylsulfamoyl-4-hydroxy-phenoxy)-5-methyl-phenyl]-malonamic acid;
N-[4-(3-cyclobutylsulfamoyl-4-hydroxy-phenoxy)-3,5-dimethyl-phenyl]-malonamic acid;
N-[4-(3-cyclopropylsulfamoyl-4-hydroxy-phenoxy)-3,5-dimethyl-phenyl]-malonamic acid;
N-[3-chloro-4-(3-cyclobutylmethanesulfonyl-4-hydroxy-phenoxy)-5-methyl-phenyl]-malonamic acid;
N-[4(3-chloro-4-(3-cyclopropylmethanesulfonyl-4-hydroxy-phenoxy)-5-methyl-phenyl]-malonamic acid;
N-[3,5-dichloro-4-(3-cyclopropylmethanesulfonyl-4-hydroxy-phenoxy)-phenyl]-malonamic acid;
N-[4-(3-cyclobutylmethanesulfonyl-4-hydroxy-phenoxy)-3,5-dimethyl-phenyl]-malonamic acid;
N-[3,5-dichloro-4-(3-cyclobutylmethanesulfonyl-4-hydroxy-phenoxy)-phenyl]-malonamic acid;
N-[4-(3-cyclopentylmethanesulfonyl-4-hydroxy-phenoxy)-3,5-dimethyl-phenyl]-malonamic acid;
N-[4-(3-cyclobutylmethanesulfonyl-4-hydroxy-phenoxy)-3,5-dimethyl-phenyl]-2-methyl-malonamic acid;
N-[3-chloro-4-(3-cyclohexylmethanesulfonyl-4-hydroxy-phenoxy)-5-methyl-phenyl]-malonamic acid;
N-[3-chloro-4-(3-cyclobutylmethanesulfonyl-4-hydroxy-phenoxy)-5-methyl-phenyl]-2-methyl-malonamic acid;
N-[3-chloro-4-(3-cyclopentylmethanesulfonyl-4-hydroxy-phenoxy)-5-methyl-phenyl]-malonamic acid;
N-[4-(3-cyclohexylmethanesulfonyl-4-hydroxy-phenoxy)-3,5-dimethyl-phenyl]-malonamic acid;
N-{4-[3-(4-fluoro-benzenesulfonyl)-4-hydroxy-phenoxy]-3,5-dimethyl-phenyl}-malonamic acid;
N-{3-chloro-4-[3-(4-fluoro-benzenesulfonyl)-4-hydroxy-phenoxy]-5-methyl-phenyl}-malonamic acid;
N-{4-[3-(4-fluoro-benzoyl)-4-hydroxy-phenoxy]-3,5-dimethyl-phenyl}-malonamic acid;
N-[4-(3-cyclopentylacetyl-4-hydroxy-phenoxy)-3,5-dimethyl-phenyl]-malonamic acid;
N-(4-{3-[(4-fluoro-phenyl)-hydroxy-methyl]-4-hydroxy-phenoxy)-3,5-dimethyl-phenyl}-malonamic acid;
N-{4-[3-(2-cyclopentyl-1-hydroxy-ethyl)-4-hydroxy-phenoxy]-3,5-dimethyl-phenyl}-malonamic acid;
N-[3-chloro-4-(3-cyclobutylmethanesulfonyl-4-hydroxy-phenoxy)-5-methyl-phenyl]-malonamic acid methyl ester;
N-[3-chloro-4-(3-cyclobutylmethanesulfonyl-4-hydroxy-phenoxy)-5-methyl-phenyl]-malonamic acid ethyl ester;
N-[4-(3-cyclobutylmethanesulfonyl-4-hydroxy-phenoxy)-3,5-dimethyl-phenyl]-malonamic acid ethyl ester;
N-[4-(3-cyclobutylmethanesulfonyl-4-hydroxy-phenoxy)-3,5-dimethyl-phenyl]-malonamic acid methyl ester;
N-[3-chloro-4-(3-cyclopentanesulfonyl-4-hydroxy-phenoxy)-5-methyl-phenyl]-malonamic acid;
N-[4-(3-cyclopentanesulfonyl-4-hydroxy-phenoxy)-3,5-dimethyl-phenyl]-malonamic acid;
N-{3-chloro-4-[3-(4-fluoro-benzenesulfonyl)-4-hydroxy-phenoxy]-5-methyl-phenyl}-malonamic acid;
N-{3-chloro-4-[3-(4-fluoro-benzenesulfonyl)-4-hydroxy-phenoxy]-5-methyl-phenyl}-malonamic acid methyl ester;
N-{3,5-dichloro-4-[3-(4-fluoro-benzenesulfonyl)-4-hydroxy-phenoxy]-phenyl}-malonamic acid methyl ester;
N-{3,5-dichloro-4-[3-(4-fluoro-benzenesulfonyl)-4-hydroxy-phenoxy]-phenyl}-malonamic acid ethyl ester;
N-{4-[3-(4-fluoro-benzenesulfonyl)-4-hydroxy-phenoxy]-3,5-dimethyl-phenyl}-2-methyl-malonamic acid methyl ester;
N-{4-[3-(4-fluoro-benzenesulfonyl)-4-hydroxy-phenoxy]-3,5-dimethyl-phenyl}-2-methyl-malonamic acid;
N-{3-chloro-4-[3-(4-fluoro-benzenesulfonyl)-4-hydroxy-phenoxy]-5-methyl-phenyl}-2-methyl-malonamic acid methyl ester;
N-{3-chloro-4-[3-(4-fluoro-benzenesulfonyl)-4-hydroxy-phenoxy]-5-methyl-phenyl}-2-methyl-malonamic acid;
N-{3,5-dichloro-4-[3-(4-fluoro-benzenesulfonyl)-4-hydroxy-phenoxy]-phenyl}-2-methyl-malonamic acid methyl ester; and
N-{3,5-dichloro-4-[3-(4-fluoro-benzenesulfonyl)-4-hydroxy-phenoxy]-phenyl}-2-methyl-malonamic acid;
N-{4-[3-(4-fluoro-benzenesulfonyl)-4-hydroxy-phenoxy]-3,5-dimethyl-phenyl}-malonamic acid; and
N-{4-[3-(4-fluoro-benzenesulfonyl)-4-hydroxy-phenoxy]-3,5-dimethyl-phenyl}-malonamic acid methyl ester; or an isomer thereof, a prodrug of said compound or isomer, or a pharmaceutically acceptable salt of said compound, isomer or prodrug.

30. A topical pharmaceutical composition for increasing the rate of nail growth which comprises an effective amount of a compound of the formula 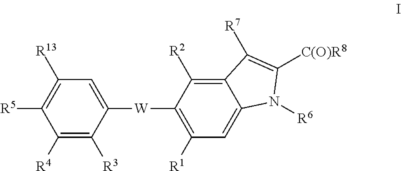
or the pharmaceutically acceptable salt thereof; wherein
W is oxygen, CH₂, CF₂, NR¹², S(O)ₘ wherein m is 0, 1 or 2;
R¹, R², and R³ are each independently selected from the group consisting of hydrogen, halo, cyano, trifluoromethyl, trifluomethoxy and (C₁-C₆)alkyl;
R⁴ is hydrogen, halo, cyano, (C₁-C₁₂)alkyl, (C₂-C₁₂)alkenyl, (C₂-C₁₂)alkynyl, (C₃-C₁₀)cycloalkyl, (C₃-C₁₀)cycloalkyl(C₁-C₆)alkyl, (C₆-C₁₀)aryl, (C₆-C₁₀)aryl(C₁-C₆)alkyl, (C₂-C₉)heteroaryl, (C₂-C₉)heteroaryl(C₁-C₆)alkyl, (C₂-C₉)heterocycloalkyl, (C₂-C₉)heterocycloalkyl(C₁-C₆)alkyl, —OR⁹, —S(O)₂NR¹⁰R¹¹, —C(O)NR¹⁰R¹¹, —C(O)R¹⁰, —CH(OH)R¹⁰, —NR¹²C(O)R¹⁰, —NR¹²C(O)NR¹⁰R¹¹, —NR¹²S(O)₂R¹⁰ or —S(O)ₙR¹⁰ wherein n is 0, 1 or 2;
R⁵ is hydroxy, fluoro, (C₁-C₄)alkoxy or —OC(O)R¹⁰;
R⁶ is hydrogen, —C(O)CH₃ or (C₁-C₆)alkyl;
R⁷ is hydrogen or (C₁-C₆)alkyl;
R⁸ is OR¹² or NR⁹R¹²;
R⁹ for each occurrence is independently hydrogen, (C₁-C₁₂)alkyl, (C₃-C₁₀)cycloalkyl, (C₂-C₉)heterocycloalkyl, (C₆-C₁₀)aryl or (C₂-C₉)heteroaryl;
R¹⁰ for each occurrence is independently hydrogen, (C₁-C₁₂)alkyl, (C₂-C₁₂)alkenyl, (C₂-C₁₂)alkynyl, (C₃-C₁₀)cycloalkyl, (C₃-C₉)cycloalkyl(C₁-C₆)alkyl, (C₆-C₁₀)aryl, (C₂-C₉)heteroaryl or halo(C₆-C₁₀)aryl;
R¹¹ for each occurrence is independently hydrogen, (C₁-C₆)alkyl, (C₃-C₁₀) cycloalkyl or (C₃-C₉)cycloalkyl(C₁-C₆)alkyl;
or R¹⁰ and R¹¹ may be taken together with the nitrogen to which they are attached to form a 3 to 10 membered heterocyclic group which may contain a second heteroatom selected from oxygen, sulfur or NR¹⁴ wherein R¹⁴ is hydrogen or (C₁-C₆)alkyl;
R¹² for each occurrence is independently hydrogen or (C₁-C₆)alkyl;
R¹³ is hydrogen, halo or (C₁-C₆)alkyl;
or R³ and R⁴ may be taken together with the carbons to which they are attached to form a compound of the formula 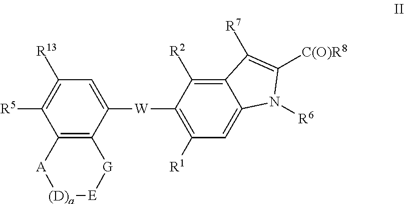
wherein a is 0, 1, 2 or 3;
A, D, E and G are each independently selected from the group consisting of CR¹⁶R¹⁷, NR¹⁸, oxygen or sulfur;
R¹⁶ and R¹⁷ for each occurrence are each independently selected from hydrogen or (C₁-C₆)alkyl; and
R¹⁸ is hydrogen, (C₁-C₆)alkyl, —C(O)R¹⁶ or —S(O)₂R¹⁰ wherein R¹⁰ is defined as above;
and a pharmaceutically acceptable carrier.

31. A composition according to claim 30 wherein the compound is selected from the group consisting of:
5-(4-Hydroxy-3-isopropyl-phenoxy)-4,6-dimethyl-1H-indole-2-carboxylic acid;
4,6-Dichloro-5-(4-hydroxy-3-isopropyl-phenoxy)-1H-indole-2-carboxylic acid;
5-(3-sec-butyl-4-hydroxy-phenoxy)-4,6-dimethyl-1H-indole-2-carboxylic acid;
5-[3-(4-Fluoro-benzyl)-4-hydroxy-phenoxyl]-4,6-dimethyl-1H-indole-2-carboxylic acid;
5-{3-[(4-Fluoro-phenyl)-hydroxy-methyl]-4-hydroxy-phenoxy}-4,6-dimethyl-1H-ondole-2-carboxylic acid;
5-[3-(2-Cyclopentyl-1-hydroxy-ethyl)-4-hydroxy-phenoxyl]-4,6-dimethyl-1H-indole-2-carboxylic acid;
5-[3-(4-Fluoro-benzoyl)-4-hydroxy-phenoxyl]-4,6-dimethyl-1H-indole-2
5-[3-(Cyclobutyl-methyl-carbamoyl)-4-hydroxy-phenoxyl]-4,6-dimethyl-1H-indole-2-carboxylic acid;
5-(3-Cyclobutylmethanesulfonyl-4-hydroxy-phenoxy)-4,6-dimethyl-1H-indole-2-carboxylic acid;
4,6-Dichloro-5-(3-cyclobutylmethanesulfonyl-4-hydroxy-phenoxy)-1H-indole-2-carboxylic acid;
4,6-Dichloro-5-(4-hydroxy-3-isopropyl-phenoxy)-1-methyl-1H-indole-2-carboxylic acid;
4,6-Dichloro-5-(4-hydroxy-3-isopropyl-phenoxy)-3-methyl-1H-indole-2-carboxylic acid;
5-[3-(4-Fluoro-benzenesulfonyl)-4-hydroxy-phenoxy]-4,6-dimethyl-1H-indole-2-carboxylic acid;
5-[3-(4-Fluoro-benzenesulfonyl)-4-hydroxy-phenoxy]-3,4,6-trimethyl-1H-indole-2-carboxylic acid;
4,6-Dichloro-5-(3-cyclobutylsulfamoyl-4-hydroxy-phenoxy)-1H-indole-2-carboxylic acid;
4-Chloro-50(3-cyclopropylsulfamoyl-4-hydroxy-phenoxy)-5-methyl-1H-indole-2-carboxylic acid;
4,6-Dichloro-5-[4-hydroxy-3-(1-isopropyl-2-methyl-propylcarbamoyl)-phenoxy]-1H-indole-2-carboxylic acid;
5-[3-(4-Fluoro-benzenesulfonyl)-4-hydroxy-phenoxy]-1,4,6-trimethyl-1H-indole-2-carboxylic acid;
5-{3-[(4-Fluoro-phenyl)-hydroxy-methyl]-4-hydroxy-phenoxy)-3,4,6-trimethyl-1H-indole-2-carboxylic acid;
5-[3-(4-Fluoro-benzyl)-4-hydroxy-phenoxy]-3,4,6-trimethyl-1H-indole-2-carboxylic acid;
5-(3-Cyclopentylmethanesulfonyl-4-hydroxy-phenoxy)-4,6-dimethyl-1H-indole-2-carboxylic acid;
4,6-Dichloro-5-(3-cyclopropylsulfamoyl-4-hydroxy-phenoxy)-1H-indole-2-carboxylic acid;
5-(3-Cyclohexylmethanesulfonyl-4-hydroxy-phenoxy)-4,6-dimethyl-1H-indole-2-carboxylic acid;
5-(3-Cyclopropylsulfamoyl-4-hydroxy-phenoxy)-4,6-dimethyl-1H-indole-2-carboxylic acid;
5-(4-Hydroxy-3-isopropyl-phenoxy)-3,4,6-trimethyl-1H-indole-2-carboxylic acid;
5-(4-Hydroxy-3-isopropyl-phenoxy)-1,4,6-trimethyl-1H-indole-2-carboxylic acid;
4,6-Dichloro-5-[3-(4-fluoro-benzenesulfonyl)-4-hydroxy-phenoxy]-1H-indole-2-carboxylic acid;
4,6-Dichloro-5-[3-(4-fluoro-benzenesulfonyl-4-hydroxy-phenoxy]-3-methyl-1H-indole-2-carboxylic acid;
5-(3-Cyclobutylsulfamoyl-4-hydroxy-phenoxy)-4,6-dimethyl-1H-indole-2-carboxylic acid;
5-[4-Hydroxy-3-(1-isopropyl-2-methyl-propylcarbamoyl)-phenoxy]-4,6-dimethyl-1H-indole-2-carboxylic acid;
4,6-Dichloro-5-{3-[(4(4-fluoro-phenyl)-hydroxy-methyl]-4-hydroxy-phenoxy)-1H-indole-2-carboxylic acid;
5-(4-Hydroxy-2,3-dimethyl-phenoxy)-4,6-dimethyl-1H-indole-2-carboxylic acid;
4,6-Dichloro-5-(4-hydroxy-2,3-dimethyl-phenoxy)-1H-indole-2-carboxylic acid;
5-(7-Hydroxy-indan-4-yloxy)-4,6-dimethyl-1H-indole-2-carboxylic acid;
4,6-Dichloro-5-(7-hydroxy-indan-4-yloxy)-1H-indole-2-carboxylic acid;
4,6-Dichloro-5-(4-hydroxy-5,6,7,8-tetrahydro-naphthalen-1-yloxy)-1H-indole-2-carboxylic acid; and
5-(4-Hydroxy-5,6,7,8-tetrahydro-naphthalen-1-yloxy)-4,6-dimethyl-1H-indole-2-carboxylic acid.

32. A composition of claim 21, 23, 24, 26, 27, 28 or 30 wherein the topical composition is in the form of a lotion, cream, ointment, paste, lacquer, artificial nail, gel, spray, aerosol, bandage or kit; and the effective amount of the compound is about 0.0001% to about 10% (w/v) of the compound per day.

33. A composition of claim 21, 23, 24, 26, 27, 28 or 30 which further comprises an effective amount of nail growth promoter, an antibacterial agent or an antifungal agent.

34. A composition of claim 33 wherein the antifungal agent is fluconazole, itraconazole, terbinafine or ciclopirox.

35. A kit for increasing nail growth in a mammal, the kit comprising:
a) a first pharmaceutical composition comprising a compound of claim 4, 6, 7, 9, 10 11 or 13;
b) a second pharmaceutical composition comprising a second compound useful for promoting the rate of nail growth, an antibacterial agent or an antifungal agent; and
c) a container.

36. A kit of claim 35 wherein the additional compound is an antifungal agent is fluconazole, itraconazole, terbinafine or ciclopirox.
